(19) United States
(12) Patent Application Publication
(10) Pub. No.: US 2024/0173369 A1
(43) Pub. Date: May 30, 2024

(54) PSEUDOMONAS BACTERIOPHAGE AND USES THEREOF

(71) Applicant: BIOMX LTD., Nes Ziona (IL)

(72) Inventors: Lior Moshe Zelcbuch, Herzeliya (IL); Rotem Edgar, Kfar-Saba (IL); Irit Sherman, Nes Ziona (IL); Dana Inbar, Ramat Gan (IL); Noa Ben-Yishai, Rehovot (IL); Maya Kahan-Hanum, Hod Hasharon (IL)

(21) Appl. No.: 18/283,909

(22) PCT Filed: Mar. 30, 2022
(86) PCT No.: PCT/IB2022/052916

§ 371 Date: Sep. 25, 2023

Related U.S. Application Data
(60) Provisional application No. 63/217,370, filed on Jul. 1, 2021.
(60) Provisional application No. 63/208,031, filed on Jun. 8, 2021.
(60) Provisional application No. 63/167,669, filed on Mar. 30, 2021.

(51) Int. Cl. A61K 35/76; A61K 31/427; A61K 31/7036; A61K 38/16; A61K 45/06; A61P 31/04; C12N 7/00

(52) U.S. Cl. CPC A61K 35/76; A61K 31/427; A61K 31/7036; A61K 38/164; A61K 45/06; A61P 31/04; C12N 7/00; C12N 2795/00032

(57) ABSTRACT

A composition comprising at least two different strains of isolated bacteriophages, each capable of infecting a bacteria of the species Pseudomonas aeruginosa, wherein at least one of said at least two different strains of isolated bacteriophages has a genomic nucleic acid sequence at least 90% identical to one of the nucleic acid sequence as set forth in SEQ ID NOs: 1-10. Uses thereof are also disclosed.

### REFERENCE TO RELATED APPLICATIONS

This application claims the benefit of the filing dates of U.S. Provisional Patent Application Nos. 63/167,669, filed on Mar. 30, 2021; 63/208,031, filed on Jun. 8, 2021; and 63/217,370, filed on Jul. 1, 2021, the entire contents of each of the above-referenced applications, including all drawings and sequence listings, are hereby incorporated herein by reference.

### SEQUENCE LISTING STATEMENT

The instant application contains a Sequence Listing which has been submitted electronically in ASCII format and is hereby incorporated by reference in its entirety. Said ASCII copy, created on Mar. 28, 2022, is named 136923-00120_SL.txt and is 1,178,072 bytes in size.

### FIELD AND BACKGROUND OF THE INVENTION

The present invention, in some embodiments thereof, relates to bacteriophage strains capable of infecting bacteria of the genus Pseudomonas and more particularly bacteria of the species Pseudomonas aeruginosa (PA).

Cystic fibrosis (CF) is the most common life-threatening autosomal recessive genetic disease in Caucasians. The estimated incidence of CF is one in 2500-4000 within the Caucasian population and holds a prevalence of about 100,000 globally (Orchard et al., 2014). Cystic fibrosis is a progressive, genetic disease that causes persistent lung infections and limits the ability to breathe over time. Pseudomonas aeruginosa is the key bacterial agent of cystic fibrosis (CF) lung infections, and the most important pathogen in progressive and severe CF lung disease. This opportunistic pathogen can grow and proliferate in patients, and exposure can occur in hospitals and other healthcare settings.

### SUMMARY OF THE INVENTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

According to an aspect of the present invention there is provided a composition comprising at least two different strains of isolated bacteriophages, each capable of infecting a bacteria of the species Pseudomonas aeruginosa, wherein at least one of the at least two different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical to one of the nucleic acid sequence as set forth in SEQ ID NOs: 1-10.

According to an aspect of the present invention there is provided a composition comprising at least two different strains of isolated bacteriophages, each capable of infecting a bacteria of the species Pseudomonas aeruginosa, wherein at least one of the at least two different strains of isolated bacteriophages has a genomic nucleic acid sequence comprising a combined region of homolog essential genes, at least 90% (e.g., at least 91%, 92%, 93%, 94%, 95%, 96%, 97%, 98%, 99%, 99.2%, 99.4%, 99.6%, 99.8%, 99.9% or 100%) identical to the combined coding region of the essential genes of a bacteriophage selected from the bacteriophages listed in Table 2, as set forth in Example 7.

According to an aspect of the present invention there is provided an isolated bacteriophage capable of infecting bacteria of the species Pseudomonas aeruginosa, wherein the bacteriophage has a genomic nucleic acid sequence at least 95% (e.g., at least 95%, 96%, 97%, 98%, 99%, 99.2%, 99.4%, 99.6%, 99.8%, 99.9% or 100%) identical to one of the nucleic acid sequences as set forth in SEQ ID NOs: 1-10.

According to an aspect of the present invention there is provided an isolated bacteriophage comprising at least 90% (e.g., at least 91%, 92%, 93%, 94%, 95%, 96%, 97%, 98%, 99%, 99.2%, 99.4%, 99.6%, 99.8%, 99.9% or 100%) identical genes (e.g., in the combined region) to the essential genes of a bacteriophage selected from the phages listed in Table 2, wherein the essential genes for the selected bacteriophage are as set forth in Example 7.

According to an aspect of the present invention, the non-essential genomic region of the selected phage comprises all regions that are not listed as essential genes for the selected bacteriophage as set forth in Example 7.

According to an aspect of the present invention there is provided a recombinant (non-wild-type) bacteriophage capable of (lytically) infecting a bacteria of the species Pseudomonas aeruginosa (e.g., Pseudomonas aeruginosa present in a Cystic Fibrosis patient), said recombinant bacteriophage has: (i) a genomic nucleic acid sequence comprising at least 90% (e.g., at least 91%, 92%, 93%, 94%, 95%, 96%, 97%, 98%, 99%, 99.2%, 99.4%, 99.6%, 99.8%, 99.9%, or 100%) identical (e.g., in the combined coding region) to the essential genes of a bacteriophage selected from the bacteriophages listed in Table 2, as set forth in Example 7, and/or (ii) at least 200 bp of said recombinant bacteriophage non-essential genomic region deleted or otherwise mutated (e.g., for eliminating mobile elements).

According to an aspect of the present invention there is provided a recombinant (non-wild-type) bacteriophage capable of (lytically) infecting a bacteria of the species Pseudomonas aeruginosa (e.g., Pseudomonas aeruginosa present in a Cystic Fibrosis patient), said recombinant bacteriophage has: (i) a genomic nucleic acid sequence at least 90% (e.g., at least 91%, 92%, 93%, 94%, 95%, 96%, 97%, 98%, 99%, 99.2%, 99.4%, 99.6%, 99.8%, or 99.9%) identical (e.g., in the combined coding region) to one of the nucleic acid sequence as set forth in SEQ ID NOs: 1-10, (ii) at least 90% (e.g., at least 91%, 92%, 93%, 94%, 95%, 96%, 97%, 98%, 99%, 99.2%, 99.4%, 99.6%, 99.8%, 99.9% or 100%) identical genes (e.g. in the combined region) to the essential genes of a bacteriophage selected from the bacteriophages listed in Table 2, as set forth in Example 7, and/or (iii) at least 200 bp of said recombinant bacteriophage non-essential genomic region deleted or otherwise mutated (e.g., for eliminating mobile elements).

According to an aspect of the present invention there is provided a recombinant (non-wild-type) bacteriophage capable of (lytically) infecting a bacteria of the species Pseudomonas aeruginosa (e.g., Pseudomonas aeruginosa present in a Cystic Fibrosis patient), said recombinant bacteriophage has: (i) a genomic nucleic acid sequence at least 90% (e.g., at least 91%, 92%, 93%, 94%, 95%, 96%, 97%, 98%, 99%, 99.2%, 99.4%, 99.6%, 99.8%, 99.9%, or 100%) identical in the combined coding region to one of the nucleic acid sequence as set forth in SEQ ID NOs: 1-10, (ii) at least 90% (e.g., at least 91%, 92%, 93%, 94%, 95%, 96%, 97%, 98%, 99%, 99.2%, 99.4%, 99.6%, 99.8%, 99.9% or 100%) identical genes (e.g., in the combined region) to the essential genes of a bacteriophage selected from the bacteriophages listed in Table 2, as set forth in Example 7, and/or (iii) at least 200 bp of said recombinant bacteriophage non-essential genomic region deleted or otherwise mutated (e.g., for eliminating mobile elements).

According to an aspect of the present invention there is provided a method of treating a disease associated with a Pseudomonas aeruginosa infection in a subject in need thereof, comprising administering to the subject a therapeutically effective amount of a composition comprising at least one isolated bacteriophage strain capable of infecting bacteria of the species Pseudomonas aeruginosa, wherein the at least one bacteriophage strain has (i) a genomic nucleic acid sequence at least 95% (e.g., at least 95%, 96%, 97%, 98%, 99%, 99.2%, 99.4%, 99.6%, 99.8%, 99.9% or 100%) identical to one of the nucleic acid sequences set forth in SEQ ID NOs: 1-10, and/or (ii) at least 90% (e.g., at least 91%, 92%, 93%, 94%, 95%, 96%, 97%, 98%, 99%, 99.2%, 99.4%, 99.6%, 99.8%, 99.9% or 100%) identical genes (e.g., in the combined region) to the essential genes of a bacteriophage selected from the bacteriophages listed in Table 2, as set forth in Example 7; thereby treating the disease associated with a Pseudomonas aeruginosa infection.

According to an aspect of the present invention there is provided a method of treating a disease associated with a Pseudomonas aeruginosa infection in a subject in need thereof, comprising administering to the subject a therapeutically effective amount of the composition described herein, thereby treating the disease associated with a Pseudomonas aeruginosa infection.

According to an aspect of the present invention there is provided a recombinant bacteriophage capable of infecting bacteria of the species Pseudomonas aeruginosa, wherein the bacteriophage has (i) a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical to one of the nucleic acid sequences as set forth in SEQ ID NOs: 1-10, and/or (ii) at least 90% (e.g., at least 91%, 92%, 93%, 94%, 95%, 96%, 97%, 98%, 99%, 99.2%, 99.4%, 99.6%, 99.8%, 99.9% or 100%) identical genes (e.g., in the combined region) to the essential genes of a bacteriophage selected from the bacteriophages listed in Table 2, as set forth in Example 7; and wherein the bacteriophage is genetically modified such that the genome thereof comprises a heterologous sequence.

According to an aspect of the present invention there is provided a pharmaceutical composition comprising the recombinant bacteriophage described herein as the active agent, and a pharmaceutical carrier.

According to an embodiment of the invention, a first of the at least two different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical to the nucleic acid sequence as set forth in SEQ ID NO: 1 (and/or comprises the essential genes of phage CF1_20Nov10 as set forth in Example 7) and a second of the at least two different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical to the nucleic acid sequence as set forth in SEQ ID NO: 2 (and/or comprises the essential genes of phage CF1_20Dec107 as set forth in Example 7).

According to an embodiment of the invention, the composition comprises at least three different strains of isolated bacteriophages, wherein a third of the at least three different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical to the nucleic acid sequence as set forth in SEQ ID NO: 3 (and/or comprises the essential genes of phage CF1_20Dec110 as set forth in Example 7).

According to an embodiment of the invention, the composition comprises a bacteriophage or a combination of bacteriophages (e.g., a combination of 2, 3, or 4 bacteriophages), selected from:
(i) a bacteriophage having a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical to SEQ ID NO: 1 (and/or comprises the essential genes of phage CF1_20Nov10 as set forth in Example 7);
(ii) a bacteriophage having a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical to SEQ ID NO: 2 (and/or comprises the essential genes of phage CF1_20Dec107 as set forth in Example 7);
(iii) a bacteriophage having a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical to SEQ ID NO: 3 (and/or comprises the essential genes of phage CF1_20Dec110 as set forth in Example 7); and/or
(iv) a bacteriophage having a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical to SEQ ID NO: 10 (and/or comprises the essential genes of phage CF1_21Oct114 as set forth in Example 7);
e.g., wherein the bacteriophage or combination of bacteriophages is capable of infecting and lysing bacteria of one or more strains of Pseudomonas aeruginosa, e.g., one or more strains of Pseudomonas aeruginosa capable of infecting a human. Optionally, at least one bacteriophage in the combination is a recombinant or engineered bacterial phage not naturally existing in nature.

In certain embodiments, the composition comprises a combination of 3 bacteriophages of (i)-(iii), such as a combination of 3 bacteriophages of (i) a bacteriophage having a genomic nucleic acid sequence of SEQ ID NO: 1 or the essential genes of phage CF1_20Nov10 as set forth in Example 7; (ii) a bacteriophage having a genomic nucleic acid sequence of SEQ ID NO: 2 or the essential genes of phage CF1_20Dec107 as set forth in Example 7; and (iii) a bacteriophage having a genomic nucleic acid sequence of SEQ ID NO: 3 or the essential genes of phage CF1_20Dec110 as set forth in Example 7.

In certain embodiments, the composition comprises a combination of 4 bacteriophages of (i)-(iv), such as a combination of 4 bacteriophages of (i) a bacteriophage having a genomic nucleic acid sequence of SEQ ID NO: 1 or the essential genes of phage CF1_20Nov10 as set forth in Example 7; (ii) a bacteriophage having a genomic nucleic acid sequence of SEQ ID NO: 2 or the essential genes of phage CF1_20Dec107 as set forth in Example 7; (iii) a bacteriophage having a genomic nucleic acid sequence of SEQ ID NO: 3 or the essential genes of phage CF1_20Dec110 as set forth in Example 7; and (iv) a bacteriophage having a genomic nucleic acid sequence of SEQ ID NO: 10 or the essential genes of phage CF1_21Oct114 as set forth in Example 7.

According to an embodiment of the invention, the at least two different strains of isolated bacteriophages in combination target at least 40, 45, 50, 55, 60 or 65 different strains of Pseudomonas aeruginosa from the list in Example 1.

According to an embodiment of the invention, the at least two different strains of isolated bacteriophages in combination target at least 25, 30, 35, 40, 45 or 50 different MLSTs of Pseudomonas aeruginosa from the list in FIG. 2.

According to an embodiment of the invention, at least 15, 17, 19, 21, 23 or 25 different strains of Pseudomonas aeruginosa from the list in Example 1 are targeted by each of the at least two different strains.

According to an embodiment of the invention, at least 9, 23, 28, 32, 35 or 36 different MLSTs of Pseudomonas aeruginosa from the list in FIG. 2 are targeted by each of the at least two different strains.

According to an embodiment of the invention, the composition comprises at least three different strains of isolated bacteriophages, each capable of infecting a bacteria of the species Pseudomonas aeruginosa, wherein each of the at least three (different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical to one of the nucleic acid sequence as set forth in SEQ ID NOs: 1-10, and/or comprises the essential genes for a bacteriophage as specified in Example 7, wherein the at least three different strains of isolated bacteriophages in combination target (i) at least 40, 45, 50, 55, 60, 65 or 70 different strains of Pseudomonas aeruginosa from the list in Example 1; and/or (ii) at least 25, 30, 35, 40, 45 or 52 different MLSTs of Pseudomonas aeruginosa from the list in FIG. 2.

According to an embodiment of the invention, the composition comprises at least three different strains of isolated bacteriophages, each capable of infecting a bacteria of the species Pseudomonas aeruginosa, wherein each of the at least three different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical to one of the nucleic acid sequence as set forth in SEQ ID Nos: 1-10, and/or comprises the essential genes for a bacteriophage as specified in Example 7, wherein (i) at least 15, 20, 25 or 30 different strains of Pseudomonas aeruginosa from the list in Example 1; and/or (ii) at least 9, 23, 28, 32, 35 or 36 different MLSTs of Pseudomonas aeruginosa from the list in FIG. 2 are targeted by each of the at least three different strains.

According to an embodiment of the invention, the at least one bacteriophage is genetically modified such that the genome thereof comprises a heterologous sequence.

According to an embodiment of the invention, the heterologous sequence encodes a therapeutic agent or a diagnostic agent.

According to an embodiment of the invention, the composition comprises no more than 10 different bacteriophage strains.

According to an embodiment of the invention, the heterologous sequence encodes a therapeutic agent or a diagnostic agent.

According to an embodiment of the invention, the therapeutic agent comprises an immune modulating agent.

According to an embodiment of the invention, the pharmaceutical composition is formulated for oral delivery or rectal delivery.

According to an embodiment of the invention, the composition is formulated for oral delivery or rectal delivery.

According to an embodiment of the invention, the disease is a Cystic Fibrosis (CF).

According to an embodiment of the invention, the administering comprises orally administering or rectally administering.

According to an embodiment of the invention, the administering comprises inhalation administering (e.g., using Meter-dosed Inhalers (MDI), Dry Powder Inhalers (DPI), Soft Mist Inhalers (SMI), Nebulizer).

According to an embodiment of the invention, the composition comprises no more than 10 different bacteriophage strains.

According to an embodiment of the invention, the method further comprises determining the strain of Pseudomonas aeruginosa colonizing the subject prior to the administering.

According to an embodiment of the invention, the at least one bacteriophage strain is genetically modified such that the genome thereof comprises a heterologous sequence.

According to an embodiment of the invention, the heterologous sequence encodes a therapeutic agent or a diagnostic agent.

According to an embodiment of the invention, the therapeutic agent comprises an immune modulating agent.

Additional aspects and embodiments of the invention described here are provided below in the numbered paragraphs.
1. A composition comprising at least two different strains of isolated bacteriophages, each capable of (lytically) infecting a bacteria of the species Pseudomonas aeruginosa (e.g., Pseudomonas aeruginosa present in a Cystic Fibrosis patient), wherein at least one of said at least two different strains of isolated bacteriophages has (i) a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to one of the nucleic acid sequence as set forth in SEQ ID NOs: 1-10, and/or (ii) at least 90% (e.g., at least 91%, 92%, 93%, 94%, 95%, 96%, 97%, 98%, 99%, 99.2%, 99.4%, 99.6%, 99.8%, 99.9% or 100%) identical genes (e.g., in the combined region) to the essential genes of a bacteriophage selected from the bacteriophages listed in Table 2, as set forth in Example 7; and
   wherein optionally, said at least two different strains of isolated bacteriophages have synergistic redundancy effect, based on either (i) time-to-mutant (TTM) that is at least 10%, 20%, 30%, 40%, 50%, 60%, 70%, or 80% above the longest individual phage TTM with respect to said bacteria, or (ii) normalized area under the curve for OD600-time plot (AUC) that is at least 10%, 20%, 30%, 40%, 50%, 60%, 70%, 80% or 90% smaller than the smallest individual phage normalized area under the curve with respect to said bacteria (or a mixture of more than one of said bacteria).
2. The composition of paragraph 1, wherein a first of said at least two different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to the nucleic acid sequence as set forth in SEQ ID NO: 1 and a second of said at least two different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to the nucleic acid sequence as set forth in SEQ ID NO: 2.
3. The composition of paragraph 2, comprising at least three different strains of isolated bacteriophages, wherein a third of said at least three different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to the nucleic acid sequence as set forth in SEQ ID NO: 3.
4. The composition of paragraph 1 or 2, comprising:
   (i) a bacteriophage having a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to SEQ ID NO: 1;
   (ii) a bacteriophage having a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to SEQ ID NO: 2;
   (iii) a bacteriophage having a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to SEQ ID NO: 3;
   (iv) a bacteriophage having a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to SEQ ID NO: 4.
5. The composition of paragraph 1, wherein said at least two different strains of isolated bacteriophages in combination target at least 40, 45, 50, 55, 60 or 65 different strains of Pseudomonas aeruginosa from the list in Example 1 of Pseudomonas aeruginosa.
6. The composition of paragraph 1 or 5, wherein at least 25 different strains of Pseudomonas aeruginosa from the list in Example 1 and/or at least 36 different MLSTs of Pseudomonas aeruginosa from the list in FIG. 2 are targeted by each of said at least two different strains.
7. The composition of paragraph 1, 5, or 6, comprising at least three different strains of isolated bacteriophages, each capable of infecting a bacteria of the species Pseudomonas aeruginosa, wherein each of said at least three different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to one of the nucleic acid sequence as set forth in SEQ ID NOs: 1-10, wherein said at least three different strains of isolated bacteriophages in combination target (i) at least 70 different strains of Pseudomonas aeruginosa from the list in Example 1; and/or (ii) at least 52 different MLSTs of Pseudomonas aeruginosa from the list in FIG. 2.
8. The composition of paragraph 1, 5, 6, or 7, comprising at least three different strains of isolated bacteriophages, each capable of infecting a bacteria of the species Pseudomonas aeruginosa, wherein each of said at least three different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to one of the nucleic acid sequence as set forth in SEQ ID NOs: 1-10, wherein (i) at least 40 different strains of Pseudomonas aeruginosa from the list in Example 1, and/or (ii) at least 52 different MLSTs of Pseudomonas aeruginosa from the list in FIG. 2 are targeted by at least two of said at least three different strains.
9. The composition of any one of paragraphs 1-8, wherein said at least one bacteriophage is genetically modified such that the genome thereof comprises a heterologous sequence.
10. The composition of paragraph 9, wherein said heterologous sequence encodes a therapeutic agent or a diagnostic agent.
11. The composition of any one of paragraphs 1-10, comprising no more than 10 different bacteriophage strains.
12. The composition of any one of paragraphs 1-11, being formulated for oral delivery, rectal delivery or delivery by inhalation.
13. A recombinant bacteriophage capable of infecting bacteria of the species Pseudomonas aeruginosa, wherein said bacteriophage has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to one of the nucleic acid sequences as set forth in SEQ ID NOs: 1-10, and wherein said bacteriophage is genetically modified such that the genome thereof comprises a heterologous sequence.
14. The recombinant bacteriophage of paragraph 13, wherein said heterologous sequence encodes a therapeutic agent or a diagnostic agent.
15. The recombinant bacteriophage of paragraph 14, or the composition of paragraph 10, wherein said therapeutic agent comprises an immune modulating agent.
16. A pharmaceutical composition comprising the recombinant bacteriophage of paragraph 13 or 14 as the active agent, and a pharmaceutical carrier.
17. The pharmaceutical composition of paragraph 16, being formulated for oral delivery, rectal delivery or delivery by inhalation.
18. An isolated bacteriophage capable of (lytically) infecting bacteria of the species Pseudomonas aeruginosa (e.g., Pseudomonas aeruginosa present in a Cystic Fibrosis patient), wherein said bacteriophage has a genomic nucleic acid sequence at least 95% (e.g., at least 95%, 96%, 97%, 98%, 99%, 99.2%, 99.4%, 99.6%, 99.8%, 99.9% or 100%) identical (e.g., in the combined coding region) to one of the nucleic acid sequences as set forth in SEQ ID NOs: 1-10.
19. A method of treating a disease associated with a Pseudomonas aeruginosa infection in a subject in need thereof (e.g., a subject having Cystic Fibrosis), comprising administering to the subject a therapeutically effective amount of a composition comprising at least one isolated bacteriophage strain capable of infecting bacteria of the species Pseudomonas aeruginosa causing the infection, wherein said at least one bacteriophage strain has a genomic nucleic acid sequence at least 95% (e.g., at least 95%, 96%, 97%, 98%, 99%, 99.2%, 99.4%, 99.6%, 99.8%, 99.9% or 100%) identical (e.g., in the combined coding region) to one of the nucleic acid sequences set forth in SEQ ID NOs: 1-10, thereby treating the disease associated with a Pseudomonas aeruginosa infection.
20. A method of treating a disease (e.g., Cystic Fibrosis) associated with a Pseudomonas aeruginosa infection in a subject in need thereof, comprising administering to the subject a therapeutically effective amount of the composition of any one of paragraphs 1-12, thereby treating the disease associated with a Pseudomonas aeruginosa infection.
21. The method of paragraph 19 or 20, wherein the disease is Cystic Fibrosis (CF).
22. The method of any one of paragraphs 19-21, wherein said administering comprises orally administering or rectally administering.
23. The method of paragraph 19, wherein said composition comprises no more than 10 different bacteriophage strains.
24. The method of any one of paragraphs 19-23, further comprising identifying the strain of Pseudomonas aeruginosa colonizing the subject prior to the administering.
25. The method of any one of paragraphs 19-24, wherein said at least one bacteriophage strain is genetically modified such that the genome thereof comprises a heterologous sequence.
26. The method of paragraph 25, wherein said heterologous sequence encodes a therapeutic agent or a diagnostic agent.
27. The method of paragraph 26, wherein said therapeutic agent comprises an immune modulating agent.
28. The method of any one of paragraphs 19-27, wherein the subject has been treated with, or is to be further treated with an antibiotic effective against Pseudomonas aeruginosa (e.g., Pseudomonas aeruginosa present in a Cystic Fibrosis patient).
29. The method of any one of paragraphs 19-27, further comprising treating the subject with an antibiotic effective against Pseudomonas aeruginosa (e.g., Pseudomonas aeruginosa present in a Cystic Fibrosis patient).
30. The method of paragraph 28 or 29, wherein the antibiotic comprises aztreonam, colistin, and/or tobramycin.

It should be understood that any one embodiment of the invention described herein, including those described only in the examples or claims, or numbered paragraphs herein, can be combined with any one or more additional embodiments of the invention, unless such combination is improper or expressly disclaimed.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a distance matrix summarizing the % sequence homology (based on local BLAST) among the isolated phages.

FIG. 2 presents the host range of the isolated phages as profiled according to the hosts bacteria multilocus sequence typing (MLST). An MLST instance where at least on bacterial member was found to be infected by the corresponding phage was marked “+.”

FIGS. 3A to 3J present growth curves of in vitro liquid infection of Pseudomonas aeruginosa strains with individual bacteriophage or cocktail, with different antibiotic or with both bacteriophage and antibiotic.

FIGS. 4A and 4B present the results of two assays used for assessing the phage effect on Pseudomonas aeruginosa biofilm and the Pseudomonas aeruginosa bacteria embedded within. FIG. 4A is an image of biofilm stained with crystal violet, after treatment with phage cocktail (right) and without it (left). FIG. 4B presents the count of bacteria embedded in the biofilm after treatment with a phage cocktail (right column), antibiotics (middle column) or non (left column).

FIGS. 5A-5G present the synergistic performance of phage mixtures in comparison to the TTM observed for each phage member separately.

### DESCRIPTION OF SPECIFIC EMBODIMENTS OF THE INVENTION

The present invention, in some embodiments thereof, relates to bacteriophage strains capable of infecting bacteria of the genus Pseudomonas and more particularly bacteria of the species Pseudomonas aeruginosa.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have isolated novel bacteriophage strains characterized by having a high specificity to one or more Pseudomonas aeruginosa strains. The disclosed bacteriophage are lytic, and as such do not have any capacity to integrate into the DNA of their bacterial host. Such bacteriophages bring about immediate target bacterial eradication through lysis after hijacking the host protein expression machinery to manufacture needed phage protein components.

The present inventors sought to combine particular phage strains and provide them as a cocktail which is capable of lysing a myriad of Pseudomonas aeruginosa strains in a single dose. The cocktails can serve as an off-the-shelf therapeutic for the treatment of Cystic fibrosis (CF), which is known to be associated with Pseudomonas aeruginosa infections. Furthermore, it is envisaged that the cocktails will have high therapeutic efficacy for treating CF at the individual level, since each individual can be infected by a wide range of Pseudomonas aeruginosa strains.

The combinations disclosed herein are typically synergistic (e.g., synergistic combination) with respect to their inhibitory effect on the target bacteria. This may be quantitated by measuring the time taken to mutation (TTM), i.e., the time taken for a bacteria to mutate and overcome the inhibitory effect of a phage. When two phages X and Y are known to infect a target bacteria strain H, the TTM of each phage separately as well as the TTM of their combination is measured under same growth conditions. The synergistic redundancy effect appears when the TTM of combination [X,Y] is longer than that of both X and Y.

In certain embodiments, the at least two different strains of isolated bacteriophages have synergistic redundancy effect, based on time-to-mutant (TTM) that is at least 10%, 20%, 30%, 40%, 50%, 60%, 70%, or 80% above the longest individual phage TTM with respect to the bacteria using which the TTM is measured.

Alternatively, or in addition, in certain embodiments, the at least two different strains of isolated bacteriophages have synergistic redundancy effect, based on normalized area under the curve for OD600-time plot (AUC) that is at least 10%, 20%, 30%, 40%, 50%, 60%, 70%, 80% or 90% smaller than the smallest individual phage normalized area under the curve with respect to said bacteria (or a mixture of more than one of said bacteria).

Here, when bacteria growth in the presence of a bacteriophage (or combination thereof) is plotted as OD600 over time, an area under the curve can be calculated for each phage (or combination thereof). Such AUC, when normalized against no phage control AUC, can be compared to assess synergistic suppression of bacteria growth by phage combinations as compared to individual phages in the combination.

Without being bound to theory, the synergy may be derived from different mechanism of infection used by the two phages X and Y. According to certain embodiments of the present invention, the synergic TTM increase may be predicted by the “at least 2 phage % coverage,” and/or the “at least 3 phage % coverage,” and/or the “at least 4 phage % coverage,” and/or the “at least 5 phage % coverage” trait of a phage combination.

Thus, according to a first aspect of the present invention, there is provided an isolated bacteriophage capable of infecting bacteria of the species Pseudomonas aeruginosa, wherein the bacteriophage has a genomic nucleic acid sequence at least 95% identical to one of the nucleic acid sequences as set forth in SEQ ID NOs: 1-10. Optionally, the bacteriophage is not naturally existing and comprises at least one heterologous engineered mutation.

As used herein, the term “bacteriophage” and “phage” are used interchangeably and refer to an isolated virus that is capable of infecting a bacterium. Typically, a phage will be characterized by: 1) the nature of the nucleic acids that make up its genome, e.g., DNA, RNA, single-stranded or double-stranded; 2) the nature of its infectivity, e.g., lytic or temperate; and 3) the particular Pseudomonas aeruginosa subspecies that it infects (and in certain instances the particular strain of that Pseudomonas aeruginosa subspecies). This aspect is known as “host range.”

As used herein, the phrases “isolated bacteriophage,” “isolate” or grammatical equivalents refer to a bacteriophage which is removed from its natural environment (e.g. removed from bacteria which it typically infects). In one embodiment, the isolated bacteriophage is removed from cellular material and/or other elements that naturally exist in the source clinical or environmental sample. The term isolated bacteriophages includes such phages isolated from human or animal patients (“clinical isolates” or “clinical variants”) and such phages isolated from the environment (“environmental isolates”).

In one embodiment, the bacteriophages are lytic.

The term “lytic bacteriophage” refers to a bacteriophage that infects a bacterial host and causes that host to lyse without incorporating the phage nucleic acids into the host genome. A lytic bacteriophage is typically not capable of reproducing using the lysogenic cycle.

As used herein, the phrase “phage strain” refers to the deposited or sequenced phage, as described herein.

The bacteriophage have been deposited at the Polish Collection of microorganisms PCM), Institute of Immunology and Experimental Therapy, Polish Academy of Sciences, U1. Weigla 12, 53-114 Wroclaw, Poland with the deposit numbers provided in Table 2.1, herein below.

The term “Pseudomonas aeruginosa” relates to a species of bacteria of the Pseudomonas genus. Pseudomonas bacterium are gram-negative, rod-shaped with unipolar motility. It will be appreciated that the term “Pseudomonas aeruginosa” includes bacteria that are currently classified or will be reclassified as Pseudomonas aeruginosa bacteria.

Exemplary strains of Pseudomonas aeruginosa that are infected by the phage strains of the present invention are those that are found in human specimens (e.g., the airway, urinary tract, burns, and wounds).

In some embodiments, the bacteriophages provided herein are capable of lysing deleterious Pseudomonas aeruginosa bacteria that induce immune and/or inflammatory response(s) and linked to progressive pulmonary function decline in CF patients among others.

In a particular embodiment, the phages described herein are capable of infecting at least one, two, three, four, five, six, seven, eight, nine or more Pseudomonas aeruginosa strains (e.g. from the list in Example 1) that infect a subject (e.g. CF patient) and/or at least one, two, three, four, five, six, seven, eight, nine or more MLSTs of Pseudomonas aeruginosa from the list in FIG. 2.

In a particular embodiment, the phages described herein are capable of infecting at least one, two, three, four, five, six, seven, eight, nine or more Pseudomonas aeruginosa strains (e.g. from the list in Example 1) and/or at least one, two, three, four, five, six, seven, eight, nine or more MLSTs of Pseudomonas aeruginosa from the list in FIG. 2 present in the subject (e.g., in the airway, urinary tract, burns, and wounds).

In a particular embodiment, the phages described herein are capable of infecting at least one, two, three, four, five, six, seven, eight, nine or more Pseudomonas aeruginosa strains (e.g., from the list in Example 1) and/or at least one, two, three, four, five, six, seven, eight, nine or more MLSTs of Pseudomonas aeruginosa from the list in FIG. 2 infecting subjects such as CF patients.

Pseudomonas spp. code for an extracellular capsule that is highly variable within the species. This capsule is a high molecular weight polysaccharide made up of different repeat units of oligosaccharides. Combinations of different oligosaccharides are referred to as serotypes. In Pseudomonas, there are over 20 serologically defined serotypes.

According to a particular embodiment, the phages described herein are capable of infecting Pseudomonas aeruginosa bacterial strains having a specific capsule locus type.

Also contemplated are progeny of the phages having a genomic nucleic acid as set forth in SEQ ID NOs: 1-10, wherein the progeny is capable of infecting the same subspecies (or even strain) of Pseudomonas aeruginosa as that the parent bacteriophage having one of the above set forth genomic nucleic acid sequence infects. Such progeny may have genomes having a sequence at least 85% identical, at least 90% identical, at least 91% identical, at least 92% identical, at least 93% identical, at least 94% identical, at least 95% identical, 96% identical, 97% identical 98% identical, or 99% identical to the genome of the parent bacteriophage.

As used herein, the term “or progeny of the bacteriophage” refers to bacteriophages stemming from or derived from the strains identified herein.

Also contemplated are functional homologs of those that have a genomic nucleic acid sequence as set forth in SEQ ID NOs: 1-10, wherein the functionally homologous bacteriophage is capable of infecting essentially the same subspecies (or even strain) of Pseudomonas aeruginosa as that which the bacteriophage having one of the above set forth genomic nucleic acid sequence infects.

As used herein “functional homolog” or “functionally homologous” or “variant” or grammatical equivalents as used herein refer to a bacteriophage with a genomic nucleic acid sequence different than that of the sequenced bacteriophage (i.e., at least one mutation) resulting in a bacteriophage that is endowed with substantially the same ensemble of biological activities (+/−10%, 20%, 40%, 50%, 60% when tested under the same conditions) as that of the sequenced bacteriophage and can be classified as infecting essentially the same strain or subspecies of bacteria based on known methods of species/strain classifications.

A bacteriophage “infects” bacteria if it either causes the bacteria to lyse or integrates its nucleic acid sequence into the bacterial genome.

According to a particular embodiment, the bacteriophage disclosed herein lyse their target bacteria.

According to a particular embodiment, the bacteriophage capability to infect (also termed “to target”) their target bacteria is measured using a solid assay or a liquid assay.

According to some embodiments, the genomic nucleic acid sequence of the bacteriophages described herein is at least about 85%, at least about 90%, at least about 91%, at least about 92%, at least about 93%, at least about 94%, at least about 95%, at least about 96% least about 97%, at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95% 99.95%, at least about 99.99%, or more identical to the (i) genomic sequence of the genomic sequences as set forth in SEQ ID NOs: 1, 2, 3, 4, 5, 6, 7, 8, 9 or 10; and/or (ii) combined region of the essential genes of a bacteriophage selected from the bacteriophages listed in Table 2, as set forth in Example 7.

In particular, the bacteriophage has a genomic nucleic acid sequence at least 95% identical (% homologous) to the nucleic acid sequence as set forth in SEQ ID NOs: 1, 2, 3, 4, 5, 6, 7, 8, 9 or 10; and/or (ii) combined region of the essential genes of a bacteriophage selected from the bacteriophages listed in Table 2, as set forth in Example 7.

According to a specific embodiment, the bacteriophage has a genomic nucleic acid sequence at least 95% identical (% homologous) to the full length nucleic acid sequence as set forth in SEQ ID NOs: 1, 2, 3, 4, 5, 6, 7, 8, 9 or 10.

According to a specific embodiment, the bacteriophage comprises at least 90% (e.g., at least 91%, 92%, 93%, 94%, 95%, 96%, 97%, 98%, 99%, 99.2%, 99.4%, 99.6%, 99.8%, 99.9% or 100%) identical genes (e.g., in the combined region) to the essential genes of a bacteriophage selected from the bacteriophages listed in Table 2, wherein the essential genes are genes as set forth for the selected bacteriophage in Example 7.

As used herein, “percent homology,” “percent identity,” “sequence identity” or “identity” or grammatical equivalents as used herein in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9].

Percent identity can be determined using any homology comparison software, including for example, the BlastN software of the National Center of Biotechnology Information (NCBI) such as by using default parameters.

Other exemplary sequence alignment programs that may be used to determine % homology or identity between two sequences include, but are not limited to, the FASTA package (including rigorous (SSEARCH, LALIGN, GGSEARCH and GLSEARCH) and heuristic (FASTA, FASTX/Y, TFASTX/Y and FASTS/M/F) algorithms, the EMBOSS package (Needle, stretcher, water and matcher), the BLAST programs (including, but not limited to BLASTN, BLASTX, TBLASTX, BLASTP, TBLASTN), megablast and BLAT. In some embodiments, the sequence alignment program is BLASTN. For example, 95% homology refers to 95% sequence identity determined by BLASTN, by combining all non-overlapping alignment segments (BLAST HSPs), summing their numbers of identical matches and dividing this sum with the length of the shorter sequence.

In some embodiments, the sequence alignment program is a basic local alignment program, e.g., BLAST. In some embodiments, the sequence alignment program is a pairwise global alignment program. In some embodiments, the pairwise global alignment program is used for protein-protein alignments. In some embodiments, the pairwise global alignment program is Needle. In some embodiments, the sequence alignment program is a multiple alignment program. In some embodiments, the multiple alignment program is MAFFT. In some embodiments, the sequence alignment program is a whole genome alignment program. In some embodiments, the whole genome alignment is performed using BLASTN. In some embodiments, BLASTN is utilized without any changes to the default parameters.

According to some embodiments of the invention, the identity is a global identity, i.e., an identity over the entire nucleic acid sequences of the invention and not over portions thereof.

According to an additional or alternative embodiment, a functional homolog is determined as the average nucleotide identity (ANI), which detects the DNA conservation of the core genome (Konstantinidis K and Tiedje J M, 2005, Proc. Natl. Acad. Sci. USA 102: 2567-2592). In some embodiments, the ANI between the functional homolog and the deposited bacteriophage (or that having a genome as set forth in any one of SEQ ID NO: 1, 2, 3, 4, 5, 6, 7, 8, 9 or 10 is of at least about 95%, at least about, 96%, at least about 97%, at least about 98%, at least about 99%, at least about 99.1%, at least about 99.5%, at least about 99.6%, at least about 99.7%, at least about 99.8%, at least about 99.9% or more.

According to an additional or alternative embodiment, a functional homolog is determined by the degree of relatedness between the functional homolog and the bacteriophage having a genome as set forth in any one of SEQ ID NO: 1, 2, 3, 4, 5, 6, 7, 8, 9 or 10 determined as the Tetranucleotide Signature Frequency Correlation Coefficient, which is based on oligonucleotide frequencies (Bohlin J. et al. 2008, BMC Genomics, 9:104). In some embodiments, the Tetranucleotide Signature Frequency Correlation coefficient between the variant and the bacteriophage having a genome as set forth in any one of SEQ ID NO: 1, 2, 3, 4, 5, 6, 7, 8, 9 or 10 is of about 0.99, 0.999 or more.

According to an additional or alternative embodiment, the degree of relatedness between the functional homolog and the bacteriophage having a genome as set forth in any one of SEQ ID NO: 1, 2, 3, 4, 5, 6, 7, 8, 9 or 10 is determined as the degree of similarity obtained when analyzing the genomes of the parent and of the variant bacteriophage by Pulsed-field gel electrophoresis (PFGE) using one or more restriction endonucleases. The degree of similarity obtained by PFGE can be measured by the Dice similarity coefficient. In some embodiments, the Dice similarity coefficient between the variant and the bacteriophage having a genome as set forth in any one of SEQ ID NO: 1, 2, 3, 4, 5, 6, 7, 8, 9 or 10 is of at least about 96%, at least about 97%, at least about 98%, at least about 99%, at least about 99.1%, at least about 99.5%, at least about 99.6%, at least about 99.7%, at least about 99.8%, at least about 99.9% or more.

According to an additional or alternative embodiment, the degree of relatedness between the functional homolog and the bacteriophage having a genome as set forth in any one of SEQ ID NO: 1, 2, 3, 4, 5, 6, 7, 8, 9 or 10 is determined by the Pearson correlation coefficient obtained by comparing the genetic profiles of both phages obtained by repetitive extragenic palindromic element-based PCR (REP-PCR) (see e.g. Chou and Wang, Int J Food Microbiol. 2006, 110:135-48). In some embodiments, the Pearson correlation coefficient obtained by comparing the REP-PCR profiles of the variant and the above described (e.g. deposited phage) is of at least about 0.99, at least about 0.999 or more—see for example bmcmicrobioldotbiomedcentraldotcom/articles/10.1186/s12866-020-01770-2.

According to an additional or alternative embodiment, the degree of relatedness between the functional homolog and the bacteriophage having a genome as set forth in any one of SEQ ID NO: 1, 2, 3, 4, 5, 6, 7, 8, 9 or 10 is defined by the linkage distance obtained by comparing the genetic profiles of both phages obtained by Multi-locus sequence typing (MLST) (see e.g. Maiden, M. C., 1998, Proc. Natl. Acad. Sci. USA 95:3140-3145). In some embodiments, the linkage distance obtained by MLST of the functional homolog and the phage having a genome as set forth in any one of SEQ ID NO: 1, 2, 3, 4, 5, 6, 7, 8, 9 or 10 is of at least about 0.99, at least about 0.999 or more.

According to an additional or alternative embodiment, the functional homolog comprises a functionally conserved gene or a fragment thereof (i.e. an essential gene) e.g., an integrase gene, a polymerase gene, a capsid protein assembly gene, a DNA terminase, a tail fiber gene, or a repressor gene that is at least about 97%, at least about 98%, at least about 99%, at least about 99.1%, at least about 99.5%, at least about 99.6%, at least about 99.7%, at least about 99.8%, at least about 99.9%, or more identical to that of the bacteriophage having a genome as set forth in any one of SEQ ID NO: 1, 2, 3, 4, 5, 6, 7, 8, 9 or 10.

For each of the disclosed bacteriophages, Example 7 provides the gene name of their essential genes.

According to an additional or alternative embodiment, the functional homolog is defined by a comparison of the coding sequence (gene) order.

According to an additional or alternative embodiment, the functional homolog is defined by a comparison of the coding sequence (gene) order of the essential genes of a bacteriophage selected from the bacteriophages listed in Table 2, as set forth in Example 7.

According to an additional or alternative embodiment, the functional homolog is defined by a comparison of order of non-coding sequences.

According to an additional or alternative embodiment, the functional homolog is defined by a comparison of order of coding and non-coding sequences.

According to some embodiments of the invention, the combined coding region of the functional homolog is such that it maintains the original order of the coding regions as within the genomic sequence of the bacteriophage having a genome as set forth in any one of SEQ ID NO: 1, 2, 3, 4, 5, 6, 7, 8, 9 or 10, yet without the non-coding regions.

For example, in case the genomic sequence has the following coding regions, A, B, C, D, E, F, G, each flanked by non-coding sequences (e.g., regulatory elements, and the like), the combined coding region will include a single nucleic acid sequence having the A+B+C+D+E+F+G coding regions combined together while maintaining the original order of their genome, yet without the non-coding sequences.

According to some embodiments of the invention, the combined non-coding region of the functional homolog is such that it maintains the original order of the non-coding regions as within the genomic sequence of the bacteriophage having a genome as set forth in any one of SEQ ID NO: 1, 2, 3, 4, 5, 6, 7, 8, 9 or 10, yet without the coding regions as originally present in the original bacteriophage.

According to some embodiments of the invention, the combined non-coding region and coding region (i.e., the genome) of the functional homolog is such that it maintains the original order of the coding and non-coding regions as within the genomic sequence of the bacteriophage having a genome as set forth in any one of SEQ ID NO: 1, 2, 3, 4, 5, 6, 7, 8, 9 or 10.

As used herein “maintains” relate to at least about 90%, 91%, 92%, 93%, 94%, 95%, 96%, 97%, 98%, 99% or 100% of the coding and/or non-coding regions of the functional homolog compared to the bacteriophage having a genome as set forth in any one of SEQ ID NO: 1, 2, 3, 4, 5, 6, 7, 8, 9 or 10.

According to an additional or alternative embodiment, the functional homolog is defined by a comparison of gene content.

According to a specific embodiment, the functional homolog comprises a combined coding region at least about 90%, at least about 91%, at least about 92%, at least about 93%, at least about 94%, at least about 95%, at least about 96%, at least about 97%, at least about 98%, at least about 99%, or more (e.g., 100%) identical to the combined coding region existing in genome of the bacteriophage having a genome as set forth in any one of SEQ ID NO: 1, 2, 3, 4, 5, 6, 7, 8, 9 or 10.

As used herein “combined coding region” refers to a nucleic acid sequence including all of the coding regions of the original bacteriophage yet without the non-coding regions of the original bacteriophage.

In one embodiment, the bacteriophages show up to 85%, 86%, 87%, 88%, 89%, 90%, 91%, 92%, 93%, 94%, 95%, 96%, 97%, 98%, 99% sequence identity with the bacteriophages disclosed herein and share at least one of the following characteristics—similar host range; similar type of infectivity (i.e. lytic or temperate).

In another embodiment, the bacteriophages show up to 85%, 86%, 87%, 88%, 89%, 90%, 91%, 92%, 93%, 94%, 95%, 96%, 97%, 98%, 99% sequence identity with the bacteriophages disclosed herein and share both of the following characteristics—similar host range; similar type of infectivity.

Additional bioinformatics methods that may be used to determine relatedness between two phage genomes include Nucmer and Minimap, both of which are alignment based tools; Win-zip, Jacard distance and MinHash, each of which are information based tools; and Codon usage similarity, pathway similarity and protein motif similarity.

As used herein, “host range” refers to the bacteria that are susceptible to infection by a particular phage. The host range of a phage may include, but is not limited to, a strain, a subspecies, a species, a genus, or multiple genera of bacteria.

Phage isolates may be prepared and phenotyped using methods known in the art, e.g., a plaque assay, liquid media assay, solid media assay. In some embodiments, the solid media assays to quantify and isolate phage are based on plaque assays (S. T. Abedon et al., Methods in Molecular Biology 2009 (Clifton, N.J.), 501, 161-74), ranging from efficiency of plating (EOP) (E. Kutter, Methods in Molecular Biology 2009 (Clifton, N.J.), 501, 141-9) to spot testing (P. Hyman et al., Advances in Applied Microbiology (1st ed., Vol. 70, pp. 217-48) 2010. Elsevier Inc.). In some embodiments, the plate format used for the plaque assay can be modified, e.g., from a petri dish to a 48-well plate.

In some embodiments, a double-layer plaque assay is used to phenotype bacteriophage isolates. For example, a starter culture of 4 mL BHIS may be inoculated with 50-100 colonies from a plate. This culture may be incubated at 37° C. for 16 hours in an anaerobic environment. A volume of 200 μL of this culture may be mixed with 100 μL of a phage-containing sample (or medium only control) and incubated for 15 minutes. 5 mL of BHIS top agar (pre-molten 0.4% agar BHIS supplemented with 1 mM Ca²⁺, Mn²⁺ and Mg²⁺ ions may be added), and the mixture may be poured over a BHIS bottom agar plate (1.5% agar BHIS). The plates may be allowed to gel at room temperature, and then incubated for 16 hours at 37° C. in anaerobic environment until plaques are identified.

In some embodiments, a modified spot drop assay is used to phenotype bacteriophage isolates. For example, a starter culture of 4 mL BHIS may be inoculated with 50-100 colonies from a plate. This culture may be incubated at 37° C. for 16 hours in an anaerobic environment. A volume of 200 μL of this culture may be mixed with 5 mL of BHIS top agar (pre-molten 0.4% agar BHIS supplemented with 1 mM Ca²⁺, Mn²⁺ and Mg²⁺ ions may be added), and the mixture may be poured over a BHIS bottom agar plate (1.5% agar BHIS). The plates may be allowed to gel at room temperature, and then incubated for 30 min at 37 C in anaerobic environment. At this stage, 5 μL of samples containing phage or media only as control may be dropped on the plate, left to absorb, and then may be incubated for 16 hours until plaques are visible for counting.

In some embodiments, a liquid media assay is used to phenotype the bacteriophage. In some embodiments, liquid-based phage infection assays follow the time-course of infection and can provide more than quantitative end-points of infection as compared to the solid-phase plaque assays. In some embodiments, by mixing phage with bacteria in liquid medium, then following the turbidity of the culture over time, one can discern finer differences (e.g., a delay in the time of cell lysis) between how different bacterial strains interact with the phage.

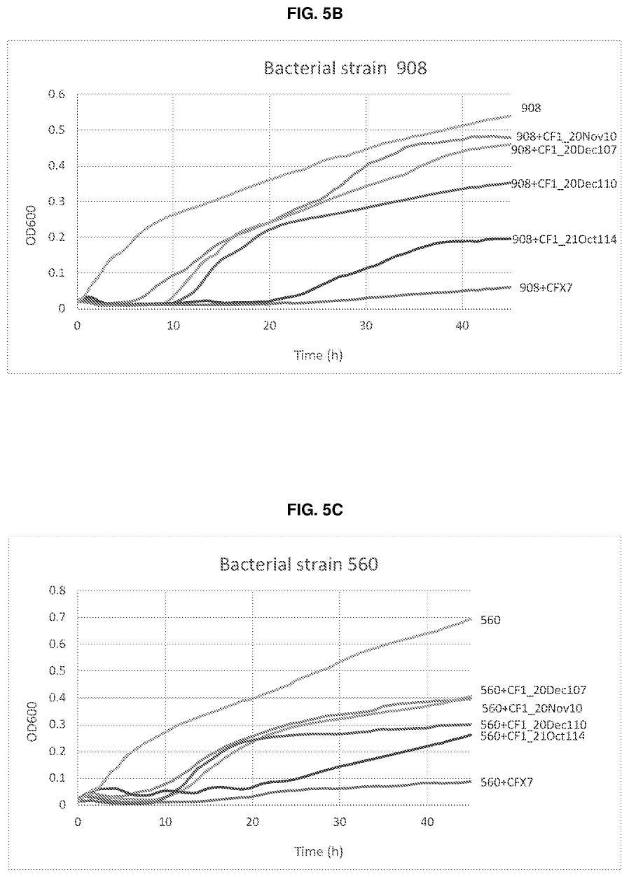

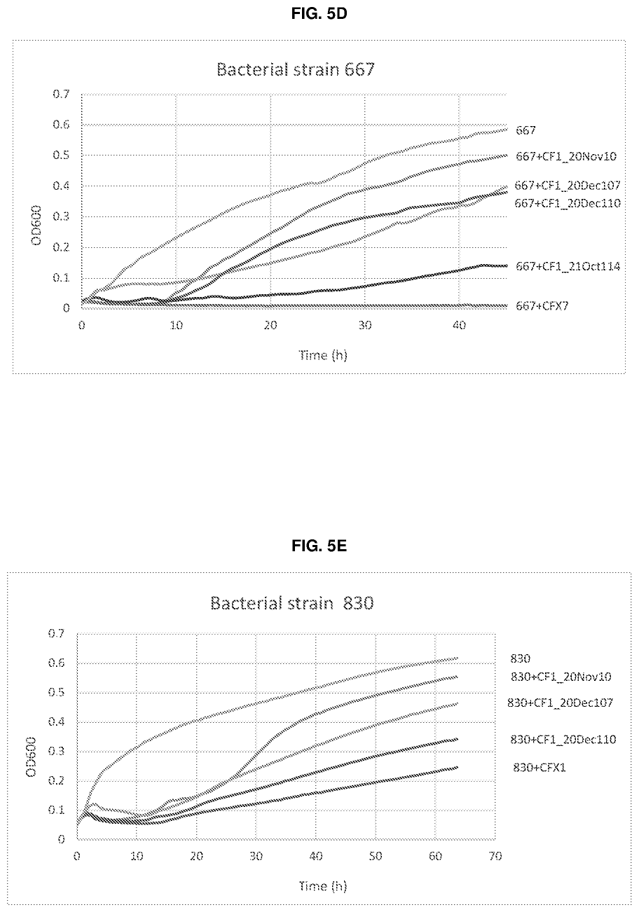

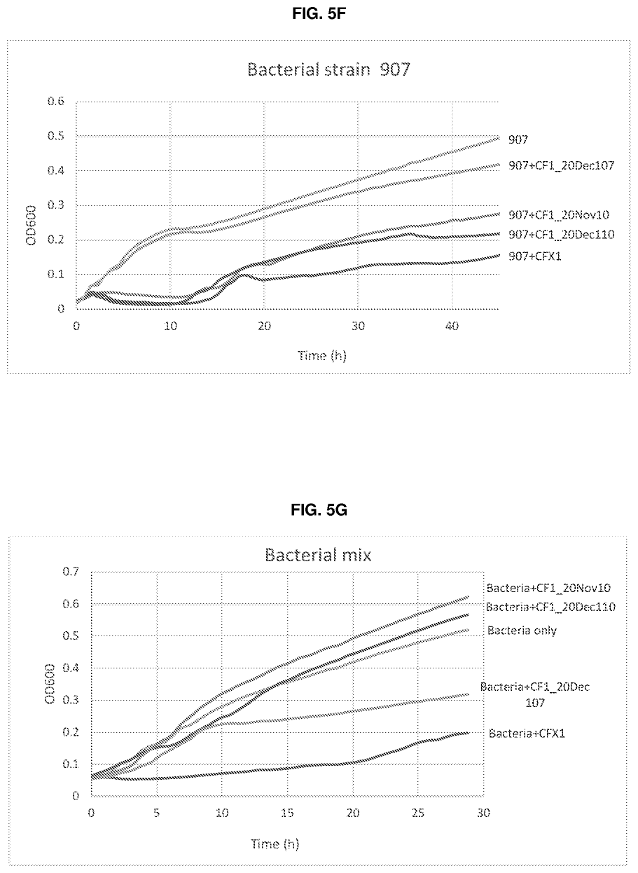

In some embodiments, a liquid-based phage infection assays is used to measure the time duration from the beginning of the experiment, when the bacteria and phages are mixed together until the host bacteria develops resistance to the phages (presumably by mutation). This period is also known as time-to-mutant (TTM). Such TTM assay was used to produce the results presented in FIGS. 5A-5G.

In some embodiments, the TTM is declared synergistic when the OD reading reaches a predetermined threshold (e.g. 0.1 OD600). Then synergistic redundancy effect is concluded if the TTM of a combination (e.g. X,Y) is for example 50% longer than the longer TTM of the individual member phages (e.g., the TTM of X by itself and the TTM of Y by itself).

In one embodiment, the synergistic effect is defined as above 10%, 20%, 30%, 40%, 50%, 60%, 70%, 80% above the longer individual phage member TTM.

In some embodiments, the liquid media assay allows for high-throughput measurements by using 96-well plates and reading optical density in a plate reader.

For example, a bacterial strain may be grown for 16 hours until an OD₆₀₀ of about 1.5-2. This culture may then be diluted using BHIS medium to a starting optical density, typically between 0.03 and 0.05 OD₆₀₀. A volume of 200 μL of culture may then be dispensed into the wells of a Nunclon flat-bottomed 96-well plate. 10 p L of a sample containing phage or 10 μL of medium as control may be added to each well. The wells may be covered with 50 μL of mineral oil to limit evaporation, and a thin sterile optically transparent polyurethane film may be added to keep the culture sterile. Optical density measurements may be carried out every 20 minutes, e.g., in a Tecan Infinite M200 plate reader connected to a Tecan EVO75 robot. Between measurements, the plate may be incubated while shaking at 37° C., e.g., inside the EVO75 incubator.

In some embodiments, infectivity is determined by the plaque presence in a solid assay only. In some embodiments, infectivity is determined by the plaque presence in a liquid assay only. In some embodiments, infectivity is determined by the plaque presence in both the liquid assay and the solid assay.

The bacteriophages described herein are typically present in a preparation in which their prevalence (i.e., concentration) is enriched over that (exceeds that) found in nature.

The term “preparation” refers to a composition in which the prevalence of bacteriophage is enriched over that found in nature. Since bacteriophages infect bacterial cells, they may be found in specimens or samples which are rich in bacteria—e.g. environmental samples such as sewage, wastewater and biological samples including feces. According to some embodiments of the invention, the preparation comprises less than 50 microbial species, e.g., bacteria and fungi—e.g., less than 40 bacterial species, less than 30 bacterial species, less than 20 bacterial species, less than 10 bacterial species, less than 5 bacterial species, less than 4 bacterial species, less than 3 bacterial species, less than 2 bacterial species or even devoid completely of bacteria.

According to a particular embodiment, the preparation comprises a single strain of bacteriophage (or a functional homolog thereof), no more than two different bacteriophage strains (or functional homologs thereof), no more than three different bacteriophage strains (or functional homologs thereof), no more than four different bacteriophage strains (or functional homologs thereof), no more than five different bacteriophage strains (or functional homologs thereof), no more than six different bacteriophage strains (or functional homologs thereof), no more than seven different bacteriophage strains (or functional homologs thereof), no more than eight different bacteriophage strains (or functional homologs thereof), no more than nine different bacteriophage strains (or functional homologs thereof), or no more than ten different bacteriophage strains (or functional homologs thereof).

In one embodiment, the preparation comprises a plurality of phage strains when at least one of the phage strains is CF1_20NOV10 (having the genome sequence at least 90% (e.g., at least 91%, 92%, 93%, 94%, 95%, 96%, 97%, 98%, 99%, 99.2%, 99.4%, 99.6%, 99.8%, 99.9% or 100%) identical to the sequence as set forth in SEQ ID NO: 1).

In another embodiment, the preparation comprises a plurality of phage strains when at least one of the phage strains is CF1_20DEC107 (having the genome sequence at least 90% (e.g., at least 91%, 92%, 93%, 94%, 95%, 96%, 97%, 98%, 99%, 99.2%, 99.4%, 99.6%, 99.8%, 99.9% or 100%) identical to the sequence as set forth in SEQ ID NO: 2).

In another embodiment, the preparation comprises a plurality of phage strains when at least one of the phage strains is CF1_20Dec110 (having the genome sequence at least 90% (e.g., at least 91%, 92%, 93%, 94%, 95%, 96%, 97%, 98%, 99%, 99.2%, 99.4%, 99.6%, 99.8%, 99.9% or 100%) identical to the sequence as set forth in SEQ ID NO: 3).

In one embodiment, the preparation comprises at least two different phage strains when at least one of the phage strains is CF1_20NOV10 (having the genome sequence at least 90% (e.g., at least 91%, 92%, 93%, 94%, 95%, 96%, 97%, 98%, 99%, 99.2%, 99.4%, 99.6%, 99.8%, 99.9% or 100%) identical to the sequence as set forth in SEQ ID NO: 1) and the other of the phage strains is CF1_20DEC107 (having the genome sequence at least 90% (e.g., at least 91%, 92%, 93%, 94%, 95%, 96%, 97%, 98%, 99%, 99.2%, 99.4%, 99.6%, 99.8%, 99.9% or 100%) identical to the sequence as set forth in SEQ ID NO: 2).

In one embodiment, the preparation comprises at least three different phage strains when at least one of the phage strains is CF1_20NOV10 (having the genome sequence at least 90% (e.g., at least 91%, 92%, 93%, 94%, 95%, 96%, 97%, 98%, 99%, 99.2%, 99.4%, 99.6%, 99.8%, 99.9% or 100%) identical to the sequence as set forth in SEQ ID NO: 1), the second of the phage strains is CF1_20DEC107 (having the genome sequence at least 90% (e.g., at least 91%, 92%, 93%, 94%, 95%, 96%, 97%, 98%, 99%, 99.2%, 99.4%, 99.6%, 99.8%, 99.9% or 100%) identical to the sequence as set forth in SEQ ID NO: 2) and the third of the phage strains is CF1_20Dec110 (having the genome sequence at least 90% (e.g., at least 91%, 92%, 93%, 94%, 95%, 96%, 97%, 98%, 99%, 99.2%, 99.4%, 99.6%, 99.8%, 99.9% or 100%) identical to the sequence as set forth in SEQ ID NO: 3).

Exemplary combinations of core phages in a single composition are provided in Table 1 herein below.

Additional contemplated combinations are provided in Example 2, Example 3 and Example 4 herein below.

TABLE 1

| | | | | Cocktail |
|---|---|---|---|---|
| Phage 1 | Phage 2 | Phage 3 | Phage 4 | name |
| CF1_20NOV10 | CF1_20DEC107 | CF1_20Dec110 | — | CFX1 |
| CF1_20NOV10 | CF1_20DEC107 | CF1_20Aug402 | — | CFX2 |
| CF1_20NOV10 | CF1_20DEC107 | CF1_20Oct199 | — | CFX3 |
| CF1_20DEC107 | CF1_20Dec110 | CF1_20Aug402 | — | CFX4 |
| CF1_20DEC107 | CF1_20Dec110 | CF1_20Oct199 | — | CFX5 |
| CF1_20Dec110 | CF1_20Aug402 | CF1_20Oct199 | — | CFX6 |
| CF1_20NOV10 | CF1_20DEC107 | CF1_20Dec110 | CF1_21Oct114 | CFX7 |
| CF1_20NOV10 | CF1_20DEC107 | CF1_20Aug402 | CF1_21Oct114 | CFX8 |
| CF1_20NOV10 | CF1_20DEC107 | CF1_20Oct199 | CF1_21Oct114 | CFX9 |
| CF1_20DEC107 | CF1_20Dec110 | CF1_20Aug402 | CF1_21Oct114 | CFX10 |
| CF1_20DEC107 | CF1_20Dec110 | CF1_20Oct199 | CF1_21Oct114 | CFX11 |
| CF1_20Dec110 | CF1_20Aug402 | CF1_20Oct199 | CF1_21Oct114 | CFX12 |

One exemplary cocktail contemplated by the present inventors is one which comprises the following phages: CF1_20NOV10, CF1_20DEC107 and CF1_20Dec110.

In one embodiment, the combination is selected such that more than 20% of the different strains of bacteria of a mixed population of Pseudomonas aeruginosa (e.g., comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 20% of all the strains of Pseudomonas aeruginosa which infect humans are targeted and lysed. In a specific embodiment, the mixed population is selected from the list Pseudomonas aeruginosa strains in Example 1.

In another embodiment, the combination is selected such that at least 40, 60, 80, 100, 200, 300, 400, 500, 600, 700, 800, 900 or 1000 different Pseudomonas aeruginosa strains are targeted.

In one embodiment, the combination is selected such that more than 30% of the different strains of bacteria of a mixed population of Pseudomonas aeruginosa (e.g. comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 30% of all the strains of Pseudomonas aeruginosa which infect humans are targeted and lysed.

In one embodiment, the combination is selected such that more than 40% of the different strains of bacteria of a mixed population of Pseudomonas aeruginosa (e.g. comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 40% of all the strains of Pseudomonas aeruginosa which infect humans are targeted and lysed.

In one embodiment, the combination is selected such that more than 45% of the different strains of bacteria of a mixed population of Pseudomonas aeruginosa (e.g. comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 45% of all the strains of Pseudomonas aeruginosa which infect humans are targeted and lysed.

In one embodiment, the combination is selected such that more than 50% of the different strains of bacteria of a mixed population of Pseudomonas aeruginosa (e.g. comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 50% of all the strains of Pseudomonas aeruginosa which infect humans are targeted and lysed.

In one embodiment, the combination is selected such that more than 55% of the different strains of bacteria of a mixed population of Pseudomonas aeruginosa (e.g. comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 55% of all the strains of Pseudomonas aeruginosa which infect humans are targeted and lysed.

In one embodiment, the combination is selected such that more than 60% of the different strains of bacteria of a mixed population of Pseudomonas aeruginosa (e.g. comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 60% of all the strains of Pseudomonas aeruginosa which infect humans are targeted and lysed.

In one embodiment, the combination is selected such that more than 65% of the different strains of bacteria of a mixed population of Pseudomonas aeruginosa (e.g. comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 65% of all the strains of Pseudomonas aeruginosa which infect humans are targeted and lysed.

In one embodiment, the combination is selected such that more than 70% of the different strains of bacteria of a mixed population of Pseudomonas aeruginosa (e.g. comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 70% of all the strains of Pseudomonas aeruginosa which infect humans are targeted and lysed.

In one embodiment, the combination is selected such that more than 75% of the different strains of bacteria of a mixed population of Pseudomonas aeruginosa (e.g. comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 75% of all the strains of Pseudomonas aeruginosa which infect humans are targeted and lysed.

In one embodiment, the combination is selected such that more than 80% of the different strains of bacteria of a mixed population of Pseudomonas aeruginosa (e.g. comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 80% of all the strains of Pseudomonas aeruginosa which infect humans are targeted and lysed.

In one embodiment, the combination is selected such that more than 85% of the different strains of bacteria of a mixed population of Pseudomonas aeruginosa (e.g. comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 85% of all the strains of Pseudomonas aeruginosa which infect humans are targeted and lysed.

In one embodiment, the combination is selected such that more than 90% of the different strains of bacteria of a mixed population of Pseudomonas aeruginosa (e.g. comprising more than 40 different Pseudomonas aeruginosa strains, more than 60 different Pseudomonas aeruginosa strains and preferably more than 80 different Pseudomonas aeruginosa strains) are targeted (and lysed). In one embodiment, the combination is selected such that more than 90% of all the strains of Pseudomonas aeruginosa which infect humans are targeted and lysed.

The combinations described herein can be selected to include phages which have overlapping host coverages. The host coverages can be defined in terms of bacterial strain classification, bacterial capsule type and/or Multi-locus sequence typing (MLST)—see (http://sanger-pathogens(dot)github(dot)io/ariba/).

In one embodiment, the combination is selected such that more than 10% of the different strains of bacteria of a mixed population of Pseudomonas aeruginosa (e.g. comprising more than 20 different Pseudomonas aeruginosa strains, more than 60 different Pseudomonas aeruginosa strains and preferably more than 80 different Pseudomonas aeruginosa strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 10% of all the strains of Pseudomonas aeruginosa which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that at least 10, 20, 40, 60, 80, 100 specific Pseudomonas aeruginosa strains are targeted by more than 1 (e.g. 2, 3, 4 or 5) phage strain of the combination.

In another embodiment, the combination is selected such that more than 15% of the different strains of bacteria of a mixed population of Pseudomonas aeruginosa (e.g. comprising more than 20 different Pseudomonas aeruginosa strains, more than 60 different Pseudomonas aeruginosa strains and preferably more than 80 different Pseudomonas aeruginosa strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 15% of all the strains of Pseudomonas aeruginosa which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 20% of the different strains of bacteria of a mixed population of Pseudomonas aeruginosa (e.g. comprising more than 20 different Pseudomonas aeruginosa strains, more than 60 different Pseudomonas aeruginosa strains and preferably more than 80 different Pseudomonas aeruginosa strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 20% of all the strains of Pseudomonas aeruginosa which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 25% of the different strains of bacteria of a mixed population of Pseudomonas aeruginosa (e.g. comprising more than 20 different Pseudomonas aeruginosa strains, more than 60 different Pseudomonas aeruginosa strains and preferably more than 80 different Pseudomonas aeruginosa strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 25% of all the strains of Pseudomonas aeruginosa which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 30% of the different strains of bacteria of a mixed population of Pseudomonas aeruginosa (e.g. comprising more than 20 different Pseudomonas aeruginosa strains, more than 60 different Pseudomonas aeruginosa strains and preferably more than 80 different Pseudomonas aeruginosa strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 30% of all the strains of Pseudomonas aeruginosa which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 35% of the different strains of bacteria of a mixed population of Pseudomonas aeruginosa (e.g. comprising more than 20 different Pseudomonas aeruginosa strains, more than 60 different Pseudomonas aeruginosa strains and preferably more than 80 different Pseudomonas aeruginosa strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 35% of all the strains of Pseudomonas aeruginosa which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 40% of the different strains of bacteria of a mixed population of Pseudomonas aeruginosa (e.g. comprising more than 20 different Pseudomonas aeruginosa strains, more than 60 different Pseudomonas aeruginosa strains and preferably more than 80 different Pseudomonas aeruginosa strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 40% of all the strains of Pseudomonas aeruginosa which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 45% of the different strains of bacteria of a mixed population of Pseudomonas aeruginosa (e.g. comprising more than 20 different Pseudomonas aeruginosa strains, more than 60 different Pseudomonas aeruginosa strains and preferably more than 80 different Pseudomonas aeruginosa strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 45% of all the strains of Pseudomonas aeruginosa which infect humans are targeted and lysed by more than one phage strain (e.g. at least 2 phage strains of the combination, at least 3 phage strains of the combination, at least 4 phage strains of the combination or at least 5 phage strains of the combination).

In another embodiment, the combination is selected such that more than 50% of the different strains of bacteria of a mixed population of Pseudomonas aeruginosa (e.g. comprising more than 20 different Pseudomonas aeruginosa strains, more than 60 different Pseudomonas aeruginosa strains and preferably more than 80 different Pseudomonas aeruginosa strains) are targeted by more than one phage strain (e.g. at least 2 phage strains of the combination, at least 3 phage strains of the combination, at least 4 phage strains of the combination or at least 5 phage strains of the combination). In one embodiment, the combination is selected such that more than 50% of all the strains of Pseudomonas aeruginosa which infect humans are targeted and lysed by more than one phage strain (e.g. at least 2 phage strains of the combination, at least 3 phage strains of the combination, at least 4 phage strains of the combination or at least 5 phage strains of the combination).

It will be appreciated that, throughout the specification, when a phage is specifically named, the present invention also considers those phage that have at least 90% identity to the sequence of their genome, wherein the phage has a similar host range.

According to a specific embodiment, the preparation comprises at least about 10⁶ PFU, 10⁷ PFU, 10⁸ PFU, 10⁹ PFU, or even 10¹⁰ PFU or more of the above described (e.g. deposited) bacteriophages or functional homolog of same or progeny of same.

The bacteriophages described herein may be genetically modified such that their genomes include a heterologous sequence.

In one embodiment, the heterologous sequence serves as a marker signifying whether transformation is successful—e.g., a barcode sequence.

In another embodiment, the heterologous sequence encodes a therapeutic or diagnostic agent (also referred to herein as a payload). The therapeutic or diagnostic agent may be a nucleic acid (e.g. RNA silencing agent), a peptide or a protein. The therapeutic agent is typically selected according to the disease which is to be treated. Thus, for example if the bacteriophage is to be used for treating diseases associated with Pseudomonas aeruginosa infection, the therapeutic agent is typically one that is known to be useful for treating that disease.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression. According to an embodiment of the invention, the RNA silencing agent is specific to the target RNA and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

Exemplary RNA silencing agents include but are not limited to siRNA, shRNA, miRNA and guide RNA (gRNA).

The therapeutic agent may be a bacterial protein or peptide (e.g., a small bacterial peptide that could act as a vaccine in the subject treated with the bacteriophage), a therapeutic protein or peptide (e.g., a cytokine, e.g., IL-15), a soluble peptide or protein ligand (e.g., a STING agonist or TRAIL), an antibody or an antibody fragment that recognizes a virulent or disease-causing antigen or is useful in an immunotherapy (e.g., a checkpoint inhibitor), an enzyme that when expressed produces a therapeutic useful product (e.g., a bacterial enzyme or metabolic cassette that produces a therapeutically useful bacterial metabolite or other bacterial antigen; a bacterial enzyme that produces LPS or causes cleavage of LPS from the outer membrane of gram negative bacteria), a shared tumor antigen or an enzyme that when expressed produces a shared tumor antigen, a unique tumor antigen or neoantigen or an enzyme that when expressed produces a unique tumor antigen or neoantigen,

In another embodiment, the therapeutic agent is an agent that is therapeutic in the treatment of cystic fibrosis.

According to another embodiment, the therapeutic agent is an immune modulating agent.

Examples of immune modulating agents include immunomodulatory cytokines, including but not limited to, IL-2, IL-15, IL-7, IL-21, GM-CSF as well as any other cytokines that are capable of further enhancing immune responses; immunomodulatory antibodies, including but not limited to, anti-CTLA4, anti-CD40, anti-41BB, anti-OX40, anti-PD1 and anti-PDL1.

Examples of diagnostic agents include fluorescent proteins or enzymes producing a colorimetric reaction. Exemplary proteins that generate a detectable signal include, but are not limited to green fluorescent protein (Genbank Accession No. AAL33912), alkaline phosphatase (Genbank Accession No. AAK73766), peroxidase (Genbank Accession No. NP_568674), histidine tag (Genbank Accession No. AAK09208), Myc tag (Genbank Accession No. AF329457), biotin ligase tag (Genbank Accession No. NP_561589), orange fluorescent protein (Genbank Accession No. AAL33917), beta galactosidase (Genbank Accession No. NM_125776), Fluorescein isothiocyanate (Genbank Accession No. AAF22695) and strepavidin (Genbank Accession No. S11540).

In another example, the diagnostic agent is a luminescent protein such as products of bacterial luciferase genes, e.g., the luciferase genes encoded by Vibrio harveyi, Vibrio fischeri, and Xenorhabdus luminescens, the firefly luciferase gene FFlux, and the like.

Recombinant methods for inserting heterologous sequences into a phage genome are well-known in the art. The appropriate coding sequence is inserted in one or more of several locations in the phage genome. In one embodiment, the nucleic acid insert that is introduced into the phage genome is approximately no more than 10% of the phage genome length.

The payload coding sequence is inserted either after early, middle or late expressing phage genes and it can be expressed as part of a phage operon, relying on either an existing phage operon, promoter and terminator, or as a distinct operon. In the latter case, a relevant promoter and terminator from the phage is inserted as part of the newly formed operon.

For example, if strong expression of a payload is required, the payload coding sequence is added after the stop codon of the major capsid protein and expressed as part of the major capsid operon. Alternatively, it can be expressed by addition of a major capsid protein promoter and terminator as an individual newly formed operon which can be inserted anywhere in the phage genome that would not damage the functionality of the phage. If low expression of a payload is desired, the payload coding sequence can be added after the terminase gene (or other low expressing gene), which usually has low expression. Moreover, payload levels are tuned by adding a ribosome binding site with a desired strength.

In order to avoid negatively affecting phage infectivity and specificity, the payload coding sequence is typically not inserted inside an existing phage open reading frame. An exception to this is the case when the payload is intended to be expressed as a fusion protein of the phage outer coat. In that latter case of payload display, the payload coding sequence is added in frame to sequence encoding the phage coat protein.

The bacteriophages described herein may be used to treat subjects having diseases associated with Pseudomonas aeruginosa infection.

Diseases associated with Pseudomonas aeruginosa infection include cystic fibrosis and infectious wounds.

As used herein, the term “subject” includes mammals, preferably human beings at any age which suffer from the pathology. Those in need of treatment may include individuals already having CF, as well as those at risk of having, or who may ultimately acquire the disease. The need for treatment is assessed, e.g., by the presence of one or more risk factors associated with the development of CF, the presence or progression of CF, or likely receptiveness to treatment of a subject having CF. For example, “treating” IBD may encompass reducing or eliminating associated symptoms, and does not necessarily encompass the elimination of the underlying disease etiology, e.g., a genetic instability locus.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

The bacteriophage may be used per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the bacteriophage accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include inhalation (e.g., by inhaler or nebulizer), topical, oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient. In one embodiment, the bacteriophage may be administered directly into the tumor of the subject.

Pharmaceutical compositions of some embodiments of the invention may be manufactured by processes well known in the art, e.g., by means of conventional mixing, dissolving, granulating, dragee-making, levigating, emulsifying, encapsulating, entrapping, spray drying, coating or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (bacteriophage) effective to prevent, alleviate or ameliorate symptoms of a disorder (e.g., inflammatory bowel disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

In some embodiments, the composition is delivered to a subject in need thereof so as to provide one or more bacteriophage in an amount corresponding to a multiplicity of infection (MOI) of about 1 to about 10. MOI is determined by assessing the approximate bacterial load in the site of infection, or using an estimate for a given type of disease and then providing phage in an amount calculated to give the desired MOI.

In some embodiments, MOI may be selected based on the “multiplicity of 10 rule,” which states that where there are on average in order of 10 phages adsorbed per bacterium, bacterial density reduces significantly (Abedon S T, 2009, Foodborne Pathog Dis 6:807-815; and Kasman L M, et al., 2002, J Virol 76:5557-5564); whereas lower-titer phage administration (e.g., using a MOI lower than 10) is unlikely to be successful (Goode D, et al., 2003, App Environ Microbiol 69:5032-5036; Kumari S, et al., 2010, J Infect Dev Ctries 4:367-377).

In other embodiments, the amount of phage (or combination of phages) is provided so as to reduce the amount of bacteria (e.g. Pseudomonas aeruginosa) present in the respiratory tract (e.g. trachea, bronchi (primary, secondary and tertiary), bronchioles (including terminal and respiratory), and lungs (including alveoli)) by at least 5%, 10%, 20%, 30%, 40%, 50%, 60%, 70%, 80%, 90% or even 100%.

In certain embodiments, the bacteriophage described herein is administered to ameliorate at least one manifestation of cystic fibrosis (CF) in a subject and results in one or more symptoms or physical parameters of the condition or disorder to improve by at least about 5%, 10%, 20%, 30%, 40%, 50%, 60%, 70%, 80%, 90%, 95%, or more as compared to levels in an untreated or control subject. In some embodiments, the improvement is measured by comparing the symptom or physical parameter in a subject prior to and following administration of the bacteriophage. In some embodiments, the measurable physical parameter is a reduction in bacterial colony-forming unit (CFU) count or plaque-forming unit (PFU) count from a sputum sample or blood sample of the subject.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics,” Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Compositions described herein may comprise more than one phage strain. In one embodiment, the composition comprises 2 phage strains, 3 phage strains, 4 phage strains, 5 phage strains or more.

In one embodiment, the bacteriophage cocktails comprise a plurality of phages that target a single Pseudomonas aeruginosa strain.

In one embodiment, the bacteriophage cocktails comprise a plurality of phages that target more than one Pseudomonas aeruginosa strain.

Examples of particular combinations of phages are provided herein below.

The pharmaceutical compositions of the present invention also may be combined with one or more non-phage therapeutic and/or prophylactic agents, useful for the treatment and/or prevention of bacterial infections, as described herein and/or known in the art (e.g. one or more traditional antibiotic agents). Other therapeutic and/or prophylactic agents that may be used in combination with the phage(s) or phage product(s) of the invention include, but are not limited to, antibiotic agents, anti-inflammatory agents, antiviral agents, antifungal agents, or local anesthetic agents.

Standard or traditional antibiotic agents that can be administered with the bacteriophages described herein include, but are not limited to, amikacin, gentamicin, kanamycin, neomycin, netilmicin, paromomycin, rhodostreptomycin, streptomycin, tobramycin, apramycin, rifamycin, naphthomycin, mupirocin, geldanamycin, ansamitocin, carbacephems, imipenem, meropenem, ertapenem, faropenem, doripenem, panipenem/betamipron, biapenem, PZ-601, cephalosporins, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, cefcapene, cefdaloxime, cefdinir, cefditoren, cefetamet, cefixime, cefmenoxime, cefteram, ceftibuten, ceftiofur, ceftiolene, ceftizoxime, ceftriaxone, cefoperazone, ceftazidime latamoxef, cefclidine, cefepime, cefluprenam, cefoselis, cefozopran, cefpirome, cefquinome, flomoxef. ceftobiprole, azithromycin, clarithromycin, dirithromycin, erythromycin, roxithromycin, aztreonam, pencillin and penicillin derivatives, actinomycin, bacitracin, colistin, polymyxin B, cinoxacin, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, stifloxacin, trovalfloxacin, prulifloxacin, acetazolamide, benzolamide, bumetanide, celecoxib, chlorthalidone, clopamide, dichlorphenamide, dorzolamide, ethoxyzolamide, furosemide, hydrochlorothiazide, indapamide, mafendide, mefruside, metolazone, probenecid, sulfacetamide, sulfadimethoxine, sulfadoxine, sulfanilamides, sulfamethoxazole, sulfasalazine, sultiame, sumatriptan, xipamide, tetracycline, chlortetracycline, oxytetracycline, doxycycline, lymecycline, meclocycline, methacycline, minocycline, rolitetracycline, methicillin, nafcillin, oxacilin, cloxacillin, vancomycin, teicoplanin, clindamycin, co-trimoxazole, flucloxacillin, dicloxacillin, ampicillin, amoxicillin and any combination thereof.

Standard antifungal agents include amphotericin B such as liposomal amphotericin B and non-liposomal amphotericin B.

The present inventors further contemplate administering to the subject a probiotic which comprises “good” bacteria to occupy the niche left by the reduced “negative” bacteria. Such probiotic bacteria may comprise lactobacillus, saccharomyces boulardii, and/or Bifidobacterium.

The bacteriophages and bacteriophage cocktails of the invention can be used in anti-infective compositions for controlling the growth of bacteria, in particular Pseudomonas aeruginosa, in order to prevent or reduce the incidence of nosocomial infections. The anti-infective compositions find use in reducing or inhibiting colonization or growth of bacterial on a surface contacted therewith. The bacteriophages of the invention may be incorporated into compositions that are formulated for application to biological surfaces, such as the skin and mucus membranes, as well as for application to non-biological surfaces.

Anti-infective formulations for use on biological surfaces include, but are not limited to, gels, creams, ointments, sprays, and the like. In particular embodiments, the anti-infective formulation is used to sterilize a surgical field, or the hands and/or exposed skin of healthcare workers and/or patients.

Anti-infective formulations for use on non-biological surfaces include sprays, solutions, suspensions, wipes impregnated with a solution or suspension and the like. In particular embodiments, the anti-infective formulation is used on solid surfaces in hospitals, nursing homes, ambulances, etc., including, e.g., appliances, countertops, and medical devices, hospital equipment. In preferred embodiments, the non-biological surface is a surface of a hospital apparatus or piece of hospital equipment. In particularly preferred embodiments, the non-biological surface is a surgical apparatus or piece of surgical equipment.

The present invention also encompasses diagnostic methods for determining the causative agent at the site of the bacterial infection. In certain embodiments, the diagnosis of the causative agent of a bacterial infection is performed by (i) culturing a sample from a patient, e.g., a sputum sample, a tumor biopsy, stool sample or other sample appropriate for culturing the bacteria causing the infection; (ii) contacting the culture with one or more bacteriophages of the invention; and (iii) monitoring for evidence of cell growth and/or lysis of the culture. Because the activity of phages tends to be species or strain specific, susceptibility, or lack of susceptibility, to one or more phages of the invention can indicate the species or strain of bacteria causing the infection.

The sample may be a tissue biopsy or swab collected from the patient, or a fluid sample, such as blood, tears, or urine.

As used herein the term “about” refers to ±10%.

The terms “comprises,” “comprising,” “includes,” “including,” “having” and their conjugates mean “including but not limited to.”

The term “consisting of” means “including and limited to.”

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a,” “an,” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology,” John Wiley and Sons, Baltimore, Maryland (1989); Perbal, “A Practical Guide to Molecular Cloning,” John Wiley & Sons, New York (1988); Watson et al., “Recombinant DNA,” Scientific American Books, New York; Birren et al. (eds) “Genome Analysis: A Laboratory Manual Series,” Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook,” Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology,” W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications,” Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1 Isolating and Characterizing Phage

### Materials and Methods

### Phage Isolation, Amplification and Determination of Phage Titers

Over 80 Pseudomonas aeruginosa bacterial strains from CF patients, were obtained from ATCC, CCUG, DSMZ, BEI and IMHA IHMA bacterial repositories, and used for isolation of infecting phage: ATCC-2192, ATCC-AB102, ATCC-AB111, ATCC-AB132, ATCC-AB145, ATCC-AB181, ATCC-AB91, BEI-EnvKY1, BEI-MX0560, BEI-PA14, BEI-PAK, CCUG 53399, CCUG 53401, CCUG 53571, CCUG 53573, CCUG 53574, CCUG 53667, CCUG 53668, CCUG 53747, CCUG 53767, CCUG 56990, CCUG 60285, CCUG-47318, DSMZ-KK1-1BAE, DSMZ-NN2-C40A, DSMZ-PAO1, DSMZ-RN3-D421, DSMZ-RP1-OC2E, DSMZ-TR1-3C2A, IHMA-2111700, IHMA-2111705, IHMA-2111714, IHMA-2111718, IHMA-2111723, IHMA-2111729, IHMA-2111733, IHMA-2111740, IHMA-2111746, IHMA-2111751, IHMA-2121752, IHMA-2121758, IHMA-2121761, IHMA-2121762, IHMA-2121764, IHMA-2121766, IHMA-2121771, IHMA-2121777, IHMA-2121781, IHMA-2121788, IHMA-2121789, IHMA-2121793, IHMA-2121797, IHMA-2121802, IHMA-2121809, IHMA-2121813, IHMA-2121816, IHMA-2121817, IHMA-2121830, IHMA-2121831, IHMA-2121833, IHMA-2121835, IHMA-2121836, IHMA-2121843, IHMA-2121877, IHMA-2121879, IHMA-2121880, IHMA-2121882, IHMA-2121883, IHMA-2121888, IHMA-2121889, IHMA-2121890, IHMA-2121894, IHMA-2121904, IHMA-2121907, IHMA-2121908, IHMA-2121910, IHMA-2121912, IHMA-2121920, IHMA-2125643, IHMA-2125647, IHMA-2125649, IHMA-2125650, IHMA-2125654, IHMA-2146665.

The phage were isolated from sewage samples after enrichment on PsA strains. The phages were amplified in liquid broth with divalent ions Mg²⁺, Mn²⁺ and Ca²⁺ (1 mM final concentration of each) and proper volume of isolated phage sample (MOI=0.01) into 4 mL log phase host culture at OD600=0.1-0.2, and incubating at 37° C., overnight. When amplified from a plaque a whole plaque was picked using a 1 μL loop and release the plaque into the culture OD600=0.1-0.2. Tubes were centrifuged and the supernatant was filtered by 0.45 μm filter.

Phage titers were determined by spot drop plaque assay as follows: host culture was prepared by inoculating 4 mL liquid BHIS with 5-10 colonies of the host and incubating at 37° C., until OD600 was 1.5 (overnight). 150 p L of host culture were added to 4 or 6 mL of molten top agar (BHIS top agar: BHIS media, 0.2% Agarose) with divalent ions Mn²⁺, Ca²⁺ and Mg²⁺ and dispensed on a Cetrimide or BHIS agar plate (1.5% Agarose). The plate was left to solidify for 15 min at RT. Then dilutions of phage sample were dropped (5 μL). Plates were incubated overnight before counting plaques (10-50 plaques/drop) and determination of phage titer (number of plaques×200×reciprocal of counted dilution=PFU/mL).

### Solid Host Range

Solid host range was performed in the same manner as detailed in the above section (“Phage isolation, amplification and determination of phage titers” section). Following plaques enumeration (10-50 plaques/drop) and determination of phage titer/host, the Efficiency of Plating (EOP) was calculated as:

$$EOP = \frac{titer on tested strain}{titer on production host}$$

For sensitive/resistant determination, EOP above 0.1 (EOP>0.1) entitled the corresponding bacteria sensitive to the respective phage. The % coverage was determined based on the number of sensitive bacteria that were found sensitive as percent of the number of bacterial strains tested.

### Liquid Host Range:

Ten bacterial colonies of each tested strain were picked and transferred into a culture tube prefilled with 4 mL of liquid BHIS. Cultures were incubated to OD600≥1.5 by shaking, 180 rpm, at 37° C. for overnight (15-16 h). Bacterial cultures were diluted using BHIS supplemented with 1 mM MMC ions to reach a final OD600 of 0.05 and dispensed into a 96-well plate. Each phage was diluted to a concentration of 10\hat{ }8 PFU/ml, and equal ratios were mixed to get cocktails combinations. Then, 10 μL of single or cocktail phages were added to the wells to a final concentration of 10\hat{ }6 PFU/well. For “no phage control” (NPC), BHIS was added to the appropriate wells. Mineral oil was added to each well to reduce evaporation of the samples, and the plates were covered with sterile film to allow bacteria growth and keep the culture sterile. Plates were incubated for 30-45 hours in a plate reader at 37° C. with shaking, and OD600 was measured every 20 minutes. Two biological repeats were performed for the assay, and BHIS media supplemented with 1 mM MMC ions served as a blank. After subtraction of the OD600 values measured in control wells with no bacteria (i.e., the optical absorbance of the medium) from the samples, the sum of all OD values measured for each treatment within the first 10 hours was calculated yielding the value of the “area under the curve” of an “OD-to-time graph” (AUC600). A bacterial strain was defined sensitive with respect to a phage if the ratio between the AUC600 treatment with the phage and the AUC600 of the no phage control (NPC) was smaller than 0.66, as presented in the following equation:

$$\frac{Phage treatment area under the curve}{NPC area under the curve} < 0.66$$

The % coverage was determined based on the number of sensitive bacteria that were found sensitive as percent of the number of bacterial strains tested.

### Results

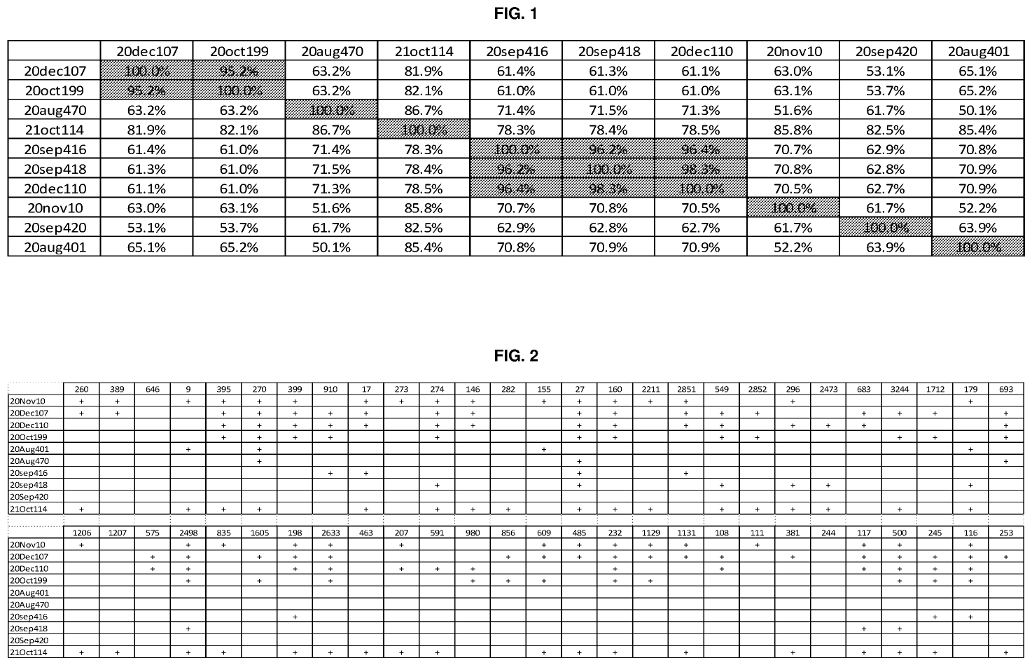

Pseudomonas aeruginosa isolates from CF patients were used to hunt phages. Phages were isolated from environmental samples (e.g., sewage and water sources), purified, and sequenced. Their taxonomy was deduced from the sequence based on International Committee on Taxonomy of Viruses (ICTV) classification (Table 2). Additionally, the sequence was used to determine the distance (sequence homology) between the phages (FIG. 1).

TABLE 2
Exemplary Isolated Bacteriophage against
Pseudomonas aeruginosa species.

| Single | | | | |
|---|---|---|---|---|
| letter | | | | SEQ |
| designa- | | | | ID |
| tion | Phage | Family | Genus | NO: |
| a | CF1_20Nov10 | Podoviridae | Bruynoghevirus | 1 |
| b | CF1_20Dec107 | Myoviridae | Pbunavirus | 2 |
| c | CF1_20Dec110 | Myoviridae | Pakpunavirus | 3 |
| d | CF1_20Oct199 | Myoviridae | Pbunavirus | 4 |
| e | CF1_20Aug401 | Autographiviridae | Phikmvvirus | 5 |
| f | CF1_20Aug470 | Podoviridae | N/A | 6 |
| g | CF1_20sep416 | Myoviridae | Pakpunavirus | 7 |
| h | CF1_20Sep418 | Myoviridae | Pakpunavirus | 8 |
| j | CF1_20Sep420 | Siphoviridae | Nipunavirus | 9 |
| l | CF1_21Oct114 | Myoviridae | Phikzvirus | 10 |

TABLE 2.1
Exemplary Isolated Bacteriophage against
Pseudomonas aeruginosa species.

| Single | | | |
|---|---|---|---|
| letter | | | |
| designation | Phage | Deposit number | Date of deposit |
| a | CF1_20Nov10 | F/00182 | 29 Mar. 2022 |
| b | CF1_20Dec107 | F/00183 | 29 Mar. 2022 |
| c | CF1_20Dec110 | F/00184 | 29 Mar. 2022 |
| d | CF1_200ct199 | F/00185 | 29 Mar. 2022 |
| e | CF1_20Aug401 | F/00186 | 29 Mar. 2022 |
| f | CF1_20Aug470 | F/00187 | 29 Mar. 2022 |
| g | CF1_20sep416 | F/00188 | 29 Mar. 2022 |
| h | CF1_20Sep418 | F/00189 | 29 Mar. 2022 |
| j | CF1_20Sep420 | NA | NA |
| l | CF1_21Oct114 | F/00190 | 29 Mar. 2022 |

The % sequence homology (based on local BLAST) of the isolated phages was compared as set forth in FIG. 1.

The host ranges (HR) of the phages were tested. HR analysis of isolated phage was performed in solid assay and liquid assay as detailed above. The percent coverage of these isolated PA strains is summarized in Table 3, herein below.

TABLE 3

| Phage | % coverage (solid) | % coverage (Liquid) |
|---|---|---|
| CF1 20Nov10 | 54% | 63% |
| CF1_20Dec107 | 60% | 64% |
| CF1_20Dec110 | 42% | 40% |
| CF1_20Oct199 | 35% | Not tested |
| CF1_20Aug401 | 12% | Not tested |
| CF1_20Aug470 | 7% | Not tested |
| CF1_20sep416 | 18% | Not tested |
| CF1_20Sep418 | 26% | Not tested |
| CF1_20Sep420 | 3% | Not tested |
| CF1_21Oct114 | Not tested | 63% |

The % coverage for cocktails CFX1 and CFX7 was measured using the liquid assay and yielded 81% and 88%, respectively. The host range of the phage was also profiled according to the multilocus sequence typing (MLST) using the Antimicrobial Resistance Identification By Assembly (ARIBA) tool (sanger-pathogens(dot)github(dot) io/ariba/). The results are set forth in FIG. 2. An MLST instance where at least on bacterial member was found to be infected by the corresponding phage was marked “+.”

### Example 2 Phage Combinations Selected According to Bacterial Coverage as Defined by Strain

For this example, the particular phages are referred to by the single letter designation in Table 1, herein above.

### 2 Phage Combinations

2 phage combinations were analyzed in silico for their ability to lyse the 85 different strains of Pseudomonas aeruginosa bacteria (as described for Example 1) based on the ability of the single phage to lyse the bacteria as assessed by a solid media assay.

The combinations are provided herein below. This trait is referred to as “at least 1 phage % coverage.” The number following each combination refers to the Percent trait performance—in this case the percent of the 85 strains that are targeted by the phage combination. The combinations are listed in descending performance grade.

Thus, for example in the case of [al;81], which provides the highest percent coverage of all the 2 phage combinations, CF1_20NOV10 and CF1_21Oct114 lysed 80% of all the strains of Pseudomonas aeruginosa analyzed. The combinations are: [al;81][bl;80][ab;80][cl;75][dl;74][ad;70][ac;69][bc;69][ah;65][bd;63][bh;62][gl;6 0][hl;58][ae;57][ag;57][el;57][af;56][fl;55][aj;55][cd;53][bg;52][be;51][bf;48][ch;45][jl;4 4][dh;38][bj;37][ce;30][cf;29][cg;27][cj;24][dg;22][fg;20][eg;18][dj;17][df;14][ej;14][gj; 14][ef;12][de;12][eh;11][gh;11][fh;5][fj;4].

The percent of host bacterial strains that are infected by two phages of a phage combination are provided. This trait is referred to as “at least 2 phage % coverage.” The phage combinations are ordered in descending performance grade.

Thus, for example, in the case of [bl;41], when CF1_20Dec107 and CF1_21Oct114 are used in a combination, 41% of the bacterial strains analyzed were targeted by both these phages. The combinations are:

[bl;41][al;38][ab;34][cl;33][bd;33][bc;32][hl;31][ac;27][dl;25][cd;23][ch;22][ad;2 0][cg;17][bh;17][dh;14][ah;14][ag;12][bg;10][gl;9][cf;7][el;7][ae;6][fl;5][eh;5][dg;5][af;4][df;4][bf;4][aj;3][bj;3][be;3][ce;3][ef;3][de;3][fg;2].

### 3 Phage Combinations

3 phage combinations were analyzed in silico for their ability to lyse the 85 different strains of Pseudomonas aeruginosa bacteria based on the ability of the single phage to lyse the bacteria as assessed by a solid media assay.

The combinations are provided herein below. The number following each combination refers to the Percent trait performance. The phage combinations are ordered in descending performance grade.

For example, in the case of [abl;88], the combination that provided the highest percent coverage, CF1_20NOV10, CF1_20Dec107 and CF1_21Oct114 lysed 88% of all the strains of Pseudomonas aeruginosa analyzed. The combinations are:

[abl;88][adl;88][acl;86][bcl;84][abc;84][abd;83][bdl;83][ahl;82][ael;82][cdl;81][a bh;80][afl;79][bhl;79][abe;78][abf;78][ajl;77][abg;77][acd;76][agl;75][chl;72][abj;72][cel;71][bcd;70][bgl;69][ach;68][bel;67][dhl;67][cfl;67][adh;67][cgl;66][egl;66][bch;65][bfl; 64][dgl;63][fgl;63][acf;63][gjl;63][acg;62][adj;62][bdh;61][adf;60][ace;60][adg;60][afg;6 0][bjl;59][djl;59][cjl;59][acj;58][ejl;57][aef;57][bce;57][ade;57][del;57][ghl;57][efl;57][b eg;56][aeg;56][dfl;55][ehl;53][bcf;53][aeh;52][fjl;52][bfg;52][bdg;52][bcg;52][aej;52][be j;51][bef;51][bde;51][agh;50][afh;50][fhl;50][cdh;50][bdf;48][agj;47][afj;47][beh;47][bhj;46][ahj;46][bdj;44][bgh;44][bgj;42][bej;42][bfh;38][cdj;34][bfj;33][cdf;31][hjl;30][cde;3 0][cef;30][cdg;30][ceh;29][cej;28][ceg;28][cfg;27][egj;23][dfg;22][cfh;22][cgh;22][chj;2 0][cgj;19][cfj;19][deg;18][efg;18][egh;17][efj;14][fgj;14][dgj;14][dej;14][def;12][deh;11][efh;11][fgh;11][dgh;11][ehj;8][ghj;8][dhj;6][dfh;5][dfj;4].

The combinations with “at least 2 phage % coverage” are provided herein below. The phage combinations are ordered in descending performance grade.

Thus, for example, in the case of [abl;65], when CF1_20NOV10, CF1_20Dec107 and CF1_21Oct114 are used in a combination, 65% of the bacterial strains analyzed were targeted by at least 2 of the 3 phages. The combinations are:

[abl;65][acl;56][bcl;56][adl;53][bdl;52][abc;49][abd;47][bcd;45][ahl;44][agl;42][c dl;42][bhl;41][acd;40][abh;40][ach;40][chl;37][bch;37][bfl;35][bgl;33][abg;32][afl;32][d hl;32][bel;32][bdh;29][adh;29][ael;28][bcg;27][abf;26][cdh;26][ajl;25][bcf;24][cgl;24][a be;24][acg;22][acf;21][ace;21][acj;20][abj;20][cfl;20][cfg;20][cdg;20][adg;20][bjl;18][bc e;15][bdg;15][bfg;15][cel;14][agj;14][afg;12][aeg;12][cdf;12][bdf;12][dfl;11][adj;10][bcj;10][bdj;10][dfg;10][adf;9][aej;9][cgj;9][ceg;9][fgl;9][dgl;9][ehl;7][cjl;7][del;7][efl;7][aef;6][ade;6][egh;5][ceh;5][deh;5][beh;5][aeh;5][efh;5][cgh;5][agh;5][ejl;5][bej;4][afj;4][bfj;4][bgj;4][egl;3][cdj;3][beg;3][cde;3][bde;3][cef;3][def;3][bef;3].

The percent of host bacterial strains that are infected by three phages of a phage combination are provided. This trait is referred to as “at least 3 phage % coverage.” The phage combinations are ordered in descending performance grade.

Thus, for example, in the case of [chl;27], when CF1_20Dec110, CF1_20Sep418 and CF1_21Oct114 are used in a combination, 27% of the bacterial strains analyzed were targeted by each of the 3 phage. The combinations are:

[chl;27][bcl;26][abl;26][bdl;25][acl;23][bcd;22][abc;22][cdl;21][bhl;20][abd;20][dhl;17][ahl;17][bch;17][adl;16][acd;15][bdh;14][cdh;14][acg;12][ach;11][abh;11][beg;10][cgl;9][adh;8][ael;7][agl;6][bgl;6][afl;5][cfl;5][bdg;5][abg;5][cdg;5][cdf;4][acf;4][bdf;4][bcf;4][bel;3][del;3][cel;3][efl;3][abj;3][cef;3][ace;3][def;3][ade;3][abe;3][dgl;3][bef;3][c de;3][bce;3][aef;3][fgl;3][bde;3][dfl;2][bfl;2][afg;2][cfg;2][adf;2][abf;2].

### 4 Phage Combinations

4 phage combinations were analyzed in silico for their ability to lyse the 85 different strains of Pseudomonas aeruginosa bacteria based on the ability of the single phage to lyse the bacteria as assessed by a solid media assay.

The combinations with the corresponding “at least 1 phage % coverage” are provided herein below. The phage combinations are ordered in descending performance grade.

For example, in the case of [abdl;91], the 4 phage combination that provided the highest percent coverage, CF1_20NOV10, CF1_20Dec107, CF1_20Oct199 and CF1_21Oct114 lysed 91% of all the strains of Pseudomonas aeruginosa analyzed. The combinations are:

[abdl;91][abcl;90][acdl;90][abhl;89][achl;86][bcdl;85][adhl;85][acel;85][abel;85][abcd;85][adjl;85][aejl;84][bchl;82][acfl;82][abfl;82][aefl;82][adel;82][abgl;81][acgl;81][a egl;81][acjl;81][abch;80][adfl;79][abdh;79][abdj;79][agjl;78][afjl;78][abde;78][adgl;78][abce;78][abef;78][afgl;78][cdhl;78][bdhl;78][abeg;78][abdf;78][abcf;78][abjl;77][abdg;7 7][abcg;77][abfg;77][aehl;76][ahjl;76][abeh;76][abej;76][abcj;75][bcel;75][begl;74][abfh;72][abgh;72][afhl;71][aghl;71][abgj;71][cefl;71][cdel;71][abfj;71][bcfl;70][acdh;70][ceg l;70][cdjl;70][bcjl;70][bdgl;69][bfgl;69][bcgl;69][cehl;69][cejl;68][egjl;68][bdel;67][befl; 67][cdfl;67][cdgl;66][degl;66][cfgl;66][bdjl;66][abhj;66][efgl;66][bcdh;64][bdfl;64][bghl;64][cfhl;64][cghl;64][dfgl;63][acdf;63][cgjl;63][cfjl;63][bgjl;63][fgjl;63][dgjl;63][acfg;6 2][acdg;62][acdj;62][eghl;61][behl;61][acde;60][acef;60][adfg;60][aceg;59][aceh;58][dej l;57][bejl;57][efjl;57][adef;57][bcde;57][bcef;57][bfhl;57][dghl;57][fghl;57][defl;57][bef g;56][adeg;56][bceg;56][aefg;56][bdeg;56][acgh;55][acfh;55][bcdj;55][bhjl;53][dehl;53][efhl;53][bcdf;53][bchj;53][begh;52][aegh;52][adeh;52][aefh;52][bceh;52][dfjl;52][bfjl;52][bdfg;52][bcfg;52][bcdg;52][acej;52][begj;52][adej;52][aefj;52][aegj;52][bcej;52][bdef; 51][ghjl;50][ehjl;50][adfh;50][adgh;50][afgh;50][dfhl;50][acfj;47][acgj;47][adfj;47][adgj; 47][afgj;47][befh;47][bdeh;47][achj;46][adhj;46][bdhj;46][chjl;46][bcfh;44][bdgh;44][bf gh;44][bcgh;44][bdej;42][bfgj;42][befj;42][bdgj;42][bcfj;42][bcgj;42][behj;41][aehj;41]1[bghj;41][fhjl;40][bdfh;38][dhjl;38][afhj;33][bfhj;33][bdfj;33][aghj;33][cdef;30][cdfg;30][cdeh;29][cegh;29][cefh;29][cdej;28][cegj;28][cefj;28][cdeg;28][cefg;28][cdhj;26][cehj;2 5][efgj;23][degj;23][cdfh;22][cdgh;22][cfgh;22][cfgj;19][cdfj;19][cdgj;19][defg;18][efgh;17][degh;17][cfhj;16][eghj;16][cghj;16][defj;14][dfgj;14][defh;11][dfgh;11][fghj;8][efhj;8][dghj;8][dehj;8].

The combinations with “at least 2 phage % coverage” are provided herein below. The phage combinations are ordered in descending performance grade.

Thus, for example, in the case of [abcl;75], when CF1_20NOV10, CF1_20Dec107, CF1_20Dec110 and CF1_21Oct114 are used in a combination, 75% of the bacterial strains analyzed were targeted by at least 2 of the 4 phage. The combinations are:

[abcl;75][abdl;70][acdl;64][bcdl;63][abhl;62][abgl;57][abcd;57][abfl;55][achl;55][abjl;51][bchl;51][abch;51][abel;50][bdhl;50][adhl;50][acfl;47][bcgl;45][abdh;44][acgl;4 2][afgl;42][adgl;42][acdh;41][bcdh;41][bcfl;41][cdhl;39][bcel;39][acel;39][abcf;39][adfl; 38][abcg;37][aegl;37][acjl;37][agjl;36][bdgl;36][bfgl;36][bdfl;35][abdg;35][abfg;35][abc e;33][adjl;33][begl;33][befl;32][bdel;32][abdf;31][bejl;31][abcj;31][acdj;31][bcdg;30][ac df;29][aghl;28][aefl;28][adel;28][abeg;28][acdg;27][bcfg;27][cfgl;27][cdgl;27][bcdf;26][abhj;26][bfjl;26][aejl;26][afjl;26][bgjl;26][bdjl;25][bcjl;25][acfg;25][abde;24][abef;24][a cej;23][behl;23][aehl;23][adfg;22][cegl;22][afhl;21][acde;21][acef;21][abdj;20][bcdj;20][cdfl;20][cdfg;20][achj;20][bcgj;19][abgj;19][acgj;19][aegj;19][acfj;19][bceg;18][aceg;18][aceh;17][abeh;17][bceh;17][bdfg;17][bcgh;16][abgh;16][cgjl;15][bhjl;15][cehl;15][bcef;15][bcde;15][adgj;14][cefl;14][abej;14][bfhl;14][bghl;14][cdel;14][afgj;14][cghl;14][bch j;13][aefg;12][adeg;12][dfgl;12][aegh;11][cegh;11][acgh;11][acfh;11][bcfh;11][abfh;11][cdjl;11][cejl;10][bcej;9][bcfj;9][abfj;9][cfgj;9][cegj;9][cdgj;9][aefj;9][adej;9][cdeg;9][cef g;9][aghj;8][cghj;8][ahjl;7][chjl;7][eghl;7][efhl;7][dehl;7][defl;7][cfhl;7][adhj;6][bdhj;6][adef;6][efgh;5][begh;5][adeh;5][cdeh;5][degh;5][befh;5][aefh;5][defh;5][bdeh;5][cefh;5][cfgh;5][adgh;5][cdgh;5][afgh;5][dejl;5][egjl;5][efjl;5][cfjl;5][bdej;4][adfj;4][bdfj;4][bdgj; 4][befj;4][begj;4][bfgj;4][degl;3][efgl;3][bdeg;3][befg;3][bdef;3][cdef;3].

The combinations with “at least 3 phage % coverage” are provided herein below. The phage combinations are ordered in descending performance grade.

Thus, for example, in the case of [abdl;40], when CF1_20NOV10, CF1_20Dec107, CF1_20Oct199 and CF1_21Oct114 are used in a combination, 40% of the bacterial strains analyzed were targeted by at least 3 of the 4 phage. The combinations are:

[abdl;40][abcl;40][achl;37][bcdl;35][abcd;34][bchl;34][acdl;33][abhl;31][abch;28][adhl;28][cdhl;28][abdh;26][bdhl;25][acgl;24][bcdh;23][acdh;23][abcg;22][abgl;21][bcfl;20][bcgl;18][abel;17][abfl;17][acdg;17][abcf;17][bcfg;15][acfl;14][acel;14][bcdg;12][acf g;12][abdg;12][abce;12][cdfl;11][bcel;10][abcj;10][abdj;10][cdfg;10][bcdf;9][acgj;9][ace g;9][adgl;9][cdgl;9][bfgl;9][cfgl;9][bdfl;8][abfg;7][bdfg;7][acjl;7][abjl;7][abdf;7][acdf;7][aefl;7][adel;7][afgl;6][bdgl;6][dfgl;6][adfl;5][acgh;5][aejl;5][abgj;4][abfj;4][abej;4][bcjl; 3][aegl;3][bdel;3][defl;3][befl;3][cdel;3][cefl;3][bceg;3][abeg;3][bcef;3][bcde;3][cdef;3][bdef;3][acef;3][abef;3][adef;3][acde;3][abde;3][adfg;2].

The percent of host strains that are infected by 4 phages of phage combinations is provided herein below. This trait is referred to herein as “at least 4 phage % coverage.” Thus, for example, in the case of [bcdl;21], when CF1_20Dec107, CF1_20Dec110, CF1_20Oct199 and CF1_21Oct114 are used in a combination, 21% of the bacterial strains tested were targeted by each of the four phage. The combinations are:

[bcdl;21][abcl;20][bchl;20][cdhl;17][bdhl;17][abdl;16][acdl;15][abcd;15][bcdh;14][achl;13][abhl;13][abch;11][adhl;10][abdh;8][acdh;8][acgl;6][bcgl;6][acfl;5][bcdg;5][ab cg;5][bcdf;4][cefl;3][defl;3][bcel;3][acel;3][cdel;3][abel;3][befl;3][adel;3][bdel;3][aefl;3][adef;3][cfgl;3][abce;3][cdef;3][bcde;3][bdgl;3][cdgl;3][bcef;3][afgl;3][bdef;3][acde;3][a bef;3][abde;3][acef;3][abgl;3][adfl;2][bcfl;2][abfl;2][cdfl;2][bdfl;2][acfg;2][abcf;2][abdf; 2][acdf;2].

### 5 Phage Combinations

5 phage combinations were analyzed in silico for their ability to lyse the 85 different strains of Pseudomonas aeruginosa bacteria based on the ability of the single phage to lyse the bacteria as assessed by a solid media assay.

The combinations with “at least 1 phage % coverage” are provided herein below (out of 118,755 possible combinations, the top 0.4% (476) are provided). The number following each combination refers to the Percent trait performance—in this case the percent of the 85 strains that are targeted by the phage combination. The phage combinations are ordered in descending performance grade.

For example, in the case of [abcdl;91], the 5 phage combination that provided the highest percent coverage, CF1_20NOV10, CF1_20Dec107, CF1_20Dec110, CF1_20Oct199 and CF1_21Oct114 lysed 91% of all the strains of Pseudomonas aeruginosa analyzed. The combinations are:

[abcdl;91][abchl;89][abdhl;89][acdhl;89][acdel;85][abcel;85][abefl;85][acefl;85][abdel;85][abdjl;85][acegl;85][abegl;85][acdjl;85][acehl;84][abehl;84][abejl;84][adejl;84][aegjl;84][acejl;84][aefjl;84][acdfl;82][abcfl;82][abdfl;82][bcdhl;82][adefl;82][abdgl;81][a cfgl;81][abfgl;81][acdgl;81][abcgl;81][abcjl;81][aefgl;81][adegl;81][aehjl;80][abcdh;79][abcdj;79][abgjl;78][abfjl;78][adfjl;78][acgjl;78][afgjl;78][adgjl;78][acfjl;78][abcde;78][a bcef;78][adfgl;78][abdef;78][acghl;78][acfhl;78][abfhl;78][abghl;78][abefg;78][abdeg;78][abceg;78][abcdf;78][abdfg;77][abcfg;77][abcdg;77][abhjl;76][achjl;76][adehl;76][aefhl; 76][aeghl;76][adhjl;76][abceh;76][abefh;76][abegh;76][abdeh;76][abcej;76][abdej;76][ab efj;76][abegj;76][bcdel;75][bcefl;75][bdegl;74][befgl;74][bcegl;74][bcdjl;74][abfgh;72][a bcfh;72][abcgh;72][abdfh;72][abdgh;72][abfgj;71][abcfj;71][adghl;71][adfhl;71][abcgj;7 1][abdfj;71][abdgj;71][cdefl;71][afghl;71][bcdfl;70][cefgl;70][cdegl;70][afhjl;70][aghjl;7 0][bcfgl;69][bcdgl;69][bdfgl;69][bcehl;69][ceghl;69][beghl;69][cefhl;69][cdehl;69][degjl;68][cefjl;68][begjl;68][cdejl;68][cegjl;68][efgjl;68][bcejl;68][bdefl;67][defgl;66][abchj;6 6][abdhj;66][abehj;66][cdfgl;66][bdghl;64][cfghl;64][bcghl;64][bcfhl;64][cdfhl;64][bfghl;64][cdghl;64][bfgjl;63][bdgjl;63][cfgjl;63][bcgjl;63][cdgjl;63][dfgjl;63][cdfjl;63][bcfjl;6 3][acdfg;62][deghl;61][befhl;61][efghl;61][bdehl;61][bchjl;61][acdef;60][cehjl;60][eghjl; 60][acdeg;59][acefg;59][acdeh;58][acefh;58][acegh;58][abghj;58][abfhj;58][bdejl;57][be fjl;57][defjl;57][bcdef;57][dfghl;57][bdfhl;57][adefg;56][bcdeg;56][bdefg;56][bcefg;56][acfgh;55][acdfh;55][acdgh;55][cdhjl;53][defhl;53][bdhjl;53][bcdhj;53][befgh;52][adefh;5 2][aefgh;52][bdegh;52][bcefh;52][bcegh;52][adegh;52][bcdeh;52][bdfjl;52][bcdfg;52][be dej;52][adefj;52][befgj;52][adegj;52][acegj;52][acefj;52][bdegj;52][bcefj;52][bcegj;52][a cdej;52][aefgj;52][cfhjl;50][cghjl;50][bghjl;50][dehjl;50][dghjl;50][efhjl;50][adfgh;50][fg hjl;50][bcehj;50][beghj;50][behjl;50][acdgj;47][acfgj;47][acdfj;47][adfgj;47][bdefh;47][a cdhj;46][bcfgh;44][bcdfh;44][bcdgh;44][bdfgh;44][bdfgj;42][bcfgj;42][bcdgj;42][bdefj;4 2][bcdfj;42][aeghj;41][adehj;41][bfghj;41][bdehj;41][bcfhj;41][bcghj;41][bdghj;41][befhj;41][aefhj;41][acehj;41][dfhjl;40][bfhjl;40][acfhj;33][bdfhj;33][adghj;33][adfhj;33][afghj;33][acghj;33][cdefh;29][cefgh;29][cdegh;29][cefgj;28][cdefj;28][cdegj;28][cdefg;28][ce fhj;25][ceghj;25][cdehj;25][defgj;23][cdfgh;22][cdfgj;19][defgh;17][efghj;16][cdghj;16][cdfhj;16][cfghj;16][deghj;16][defhj;8][dfghj;8].

The combinations with “at least 2 phage % coverage” are provided herein below. The phage combinations are ordered in descending performance grade.

Thus, for example, in the case of [abcdl;78], when CF1_20NOV10, CF1_20Dec107, CF1_20Dec110, CF1_20Oct199 and CF1_21Oct114 are used in a combination, 78% of the bacterial strains tested were targeted by at least 2 of the 5 phage. The combinations are:

[abcdl;78][abchl;68][abcfl;64][abdhl;64][abcgl;63][abcel;60][abdgl;60][abfgl;60][abcjl;59][acdhl;57][bcdhl;57][abdfl;55][abegl;55][abcdh;52][abgjl;52][abdjl;51][abdel;50][abefl;50][acdjl;48][acdfl;47][adfgl;45][acdgl;45][bcdgl;45][acfgl;45][bcfgl;45][bcegl;4 4][abfjl;42][abejl;42][bcdfl;41][bcdel;39][acdel;39][acefl;39][bcefl;39][abcdf;39][abhjl;3 8][abcfg;37][abcdg;37][aefgl;37][acegl;37][adegl;37][bcdjl;37][aegjl;36][afgjl;36][acgjl; 36][adgjl;36][bcgjl;36][acejl;36][acfjl;36][bdfgl;36][abghl;35][abdfg;35][abcdj;34][befgl; 33][abcde;33][abchj;33][abcef;33][bdegl;33][bdefl;32][begjl;31][bcejl;31][bdejl;31][befjl;31][abceg;31][acehl;30][bcehl;30][abehl;30][aeghl;30][bcdfg;30][abceh;29][bcghl;28][a defl;28][acfhl;28][adghl;28][abfhl;28][acghl;28][afghl;28][abdeg;28][abefg;28][acdfg;27][cdfgl;27][acdhj;26][abdhj;26][adfjl;26][adejl;26][bfgjl;26][aefjl;26][bdgjl;26][bdfjl;26][bcfjl;26][abdef;24][acegj;23][acdej;23][abegj;23][abcej;23][acefj;23][abegh;23][bcegh;2 3][bdhjl;23][befhl;23][bchjl;23][beghl;23][ceghl;23][adehl;23][achjl;23][bdehl;23][aefhl; 23][abcfh;22][cefgl;22][abcgh;22][cdegl;22][adfhl;21][bcfhl;21][acdef;21][cegjl;21][bed hj;20][aghjl;20][bghjl;20][bfhjl;20][cghjl;20][behjl;20][abcfj;19][bcdgj;19][abfgj;19][aef gj;19][adegj;19][acdgj;19][acfgj;19][abdgj;19][abcgj;19][bcegj;19][acdfj;19][bcfgj;19][b cefg;18][bcdeg;18][acdeg;18][acefg;18][bcefh;17][acdeh;17][abdeh;17][abefh;17][bcdeh; 17][acefh;17][acegh;17][bcfgh;16][acghj;16][abfgh;16][bcdgh;16][abdgh;16][abghj;16][b cghj;16][acfhj;16][acehj;16][cfgjl;15][cdgjl;15][cefhl;15][adhjl;15][cdehl;15][bcdef;15][b dghl;14][abefj;14][abdej;14][bfghl;14][adfgj;14][cfghl;14][cdghl;14][cdefl;14][bdfh;14][adefg;12][adegh;11][aefgh;11][cefgh;11][cdegh;11][acdfh;11][bcdfh;11][abdfh;11][acfgh;11][acdgh;11][cdejl;10][cefjl;10][afhjl;10][aehjl;10][cfhjl;10][cehjl;10][bcdfj;9][cdegj;9][adefj;9][bcefj;9][abdfj;9][cdfgj;9][cefgj;9][bcdej;9][cdefg;9][bcehj;8][ceghj;8][cfghj;8][abfhj;8][abehj;8][cdghj;8][bcfhj;8][adghj;8][afghj;8][aeghj;8][efghl;7][cdhjl;7][deghl;7][defhl;7][cdfhl;7][befgh;5][bdefh;5][adefh;5][bdegh;5][cdefh;5][defgh;5][adfgh;5][cdfgh; 5][defjl;5][cdfjl;5][efgjl;5][degjl;5][bdegj;4][befgj;4][bdefj;4][bdfgj;4][defgl;3][bdefg;3].

The combinations with “at least 3 phage % coverage” are provided herein below. The phage combinations are ordered in descending performance grade.

Thus, for example, in the case of [abcdl;47], when CF1_20NOV10, CF1_20Dec107, CF1_20Dec110, CF1_20Oct199 and CF1_21Oct114 are used in a combination, 47% of the bacterial strains tested were targeted by at least 3 of the 5 phage. The combinations are:

[abcdl;47][abchl;44][abdhl;39][acdhl;39][bcdhl;35][abcdh;32][abcdg;27][abcgl;2 7][abcfl;26][abcfg;25][abcdf;24][acdgl;24][acfgl;24][abdfl;23][acegl;22][abcel;21][abfgl; 21][bcdgl;21][abdgl;21][bcfgl;21][acdfl;20][bcdfl;20][acdfg;20][abcgj;19][abegl;18][abef l;17][abdel;17][abdfg;17][abcdj;17][abejl;15][acgjl;15][abceg;15][bcdfg;15][abdjl;14][ab cjl;14][acdel;14][acghl;14][acefl;14][abchj;13][abcde;12][bdfgl;12][abcef;12][cdfgl;12][a bcgh;11][bcegl;11][bcefl;10][bcdel;10][acejl;10][abfjl;10][abgjl;10][acdgj;9][abcfj;9][abc ej;9][acfgj;9][acegj;9][acdeg;9][acefg;9][adfgl;9][acghj;8][bcehl;7][abehl;7][achjl;7][bchj l;7][acehl;7][abhjl;7][acdjl;7][bcfhl;7][bcghl;7][adefl;7][abghl;7][acfhl;7][abfhl;7][abdhj; 6][acegh;5][abceh;5][acfgh;5][acdgh;5][abcfh;5][acfjl;5][bcgjl;5][aefjl;5][adejl;5][aegjl;5][bcejl;5][bcfjl;5][abfgj;4][abegj;4][abdej;4][abdfj;4][abdgj;4][abefj;4][bcdjl;3][adegl;3][aefgl;3][cdefl;3][bdefl;3][abdeg;3][bcefg;3][bcdeg;3][abefg;3][bcdef;3][abdef;3][acdef;3]

The combinations with the highest “at least 4 phage % coverage” are provided herein below. The phage combinations are ordered in descending performance grade.

Thus, for example, in the case of [abcdl;30], when CF1_20NOV10, CF1_20Dec107, CF1_20Dec110, CF1_20Oct199 and CF1_21Oct114 are used in a combination, 30% of the bacterial strains analyzed were targeted by at least 4 of the 5 phage. The combinations are:

[abcdl;30][abchl;27][abdhl;25][bcdhl;25][acdhl;25][abcdh;23][abcgl;18][abcfl;14][abcel;10][abcdg;10][acdgl;9][bcfgl;9][bcdfl;8][abcfg;7][bcdfg;7][abdgl;6][acfgl;6][abfgl;6][bcdgl;6][cdfgl;6][acdfl;5][abcdf;4][abcjl;3][abefl;3][acdel;3][cdefl;3][acefl;3][bdefl;3][adefl;3][abdel;3][bcdel;3][bcefl;3][abceg;3][bdfgl;3][adfgl;3][acdef;3][abcde;3][bcdef;3][abdef;3][abcef;3][abdfl;2][acdfg;2].

### Example 3 Phage Combinations Selected According to Host Coverage as Classified by Bacterial MLST

For this example, the particular phages are referred to by the single letter designation in Table 1, herein above.

### 2 Phage Combinations

2 phage combinations were analyzed in silico for their ability to lyse the 85 different strains of Pseudomonas aeruginosa bacteria as classified by MLST, based on the ability of the single phage to lyse a bacteria of a particular MLST as assessed by a solid media assay.

The combinations and % of bacterial MLSTs are provided herein below. This trait is referred to as “at least 1 phage % coverage.” The number following each combination refers to the Percent trait performance—in this case the percent of bacteria MLSTs that are targeted by the phage combination. The combinations are listed in descending performance grade. The combinations are:

[bl;91][al;89][dl;87][cl;85][ab;85][gl;78][ad;77][ac;77][el;76][ah;76][bh;76][bc;7 5][hl;75][fl;73][ag;72][bd;68][af;68][be;66][aj;66][ae;66][cd;64][ch;61][jl;60][dh;57][bf; 56][bg;56][ce;55][cf;48][cg;48][bj;40][gh;40][eg;38][eh;37][fg;36][dg;36][fh;30][de;27][cj;26][df;24][ef;22][dj;20][ej;20][gj;20].

The percent of host bacterial MLSTs that are infected by two phages of a phage combination are provided. This trait is referred to as “at least 2 phage % coverage.” The phage combinations are ordered in descending performance grade. The combinations are:

[al;50][bl;47][hl;45][bc;42][ab;40][bd;40][cl;39][ch;38][ac;33][ah;33][cd;29][dl;2 9][bh;28][cg;28][bg;28][ad;24][dh;23][ae;22][ag;20][el;17][gl;17][dg;16][eh;12][bf;12][c f;12][df;12][gh;10][fh;10][fl;8][af;8][de;5][ef;5][be;5][ce;5][fg;4].

### 3 Phage Combinations

3 phage combinations were analyzed in silico for their ability to lyse the 85 different strains of Pseudomonas aeruginosa bacteria as classified by MLST, based on the ability of the single phage to lyse a bacteria of a particular MLST as assessed by a solid media assay.

The combinations and % of bacterial MLSTs are provided herein below. This trait is referred to as “at least 1 phage % coverage.” The number following each combination refers to the Percent trait performance—in this case the percent of bacteria MLSTs that are targeted by the phage combination. The combinations are listed in descending performance grade. The combinations are:

[abl;97][adl;97][acl;95][afl;95][bhl;95][bel;94][cel;94][ael;94][bcl;93][bdl;93][ajl; 93][cdl;91][agl;91][abc;90][ahl;90][egl;88][abg;88][abf;88][ehl;87][abd;87][acd;87][bfl;8 6][cgl;86][cfl;86][bgl;86][abh;85][dhl;85][chl;85][abe;83][dgl;82][fgl;82][del;82][adh;80][ach;80][ejl;80][fhl;80][acg;80][djl;80][ghl;80][bjl;80][abj;80][gjl;80][acf;80][dfl;78][efl;76][bch;76][bdh;76][adf;76][afg;76][adg;76][bcd;75][beh;75][cjl;73][adj;73][acj;73][bce;72][ace;72][afh;70][agh;70][fjl;70][aeg;66][cdh;66][bef;66][beg;66][aef;66][ade;66][bde;66][aeh;62][ceh;62][aej;60][hjl;60][bgh;60][afj;60][agj;60][ahj;60][bcf;60][bcg;60][bej; 60][bfh;60][bdf;56][bdg;56][bfg;56][ceg;55][cef;55][cde;55][bcj;53][egh;50][cgh;50][cfh;50][cfg;48][cdf;48][cdg;48][bdj;46][bgj;40][egj;40][fgh;40][cej;40][dgh;40][bfj;40][bhj; 40][efg;38][deg;38][efh;37][deh;37][dfg;36][cdj;33][dfh;30][dej;30][def;27][ehj;25][ghj; 25][efj;20][fgj;20][cfj;20][cgj;20][chj;20][dgj;20][dfj;10].

The percent of host bacterial MLSTs that are infected by two phages of a phage combination are provided below. This trait is referred to as “at least 2 phage % coverage.” The phage combinations are ordered in descending performance grade. The combinations are:

[abl;72][bcl;66][ahl;65][adl;64][acl;64][abh;61][agl;60][bdl;60][bhl;60][bcd;57][a bc;57][abd;57][ach;57][bch;57][chl;55][cdl;54][ael;52][ace;50][dhl;50][abe;50][afl;47][b gl;47][cdh;47][adh;47][bel;47][acd;46][bcf;44][bcg;44][bfl;43][bdh;42][abg;40][ghl;40][acg;40][agh;40][cgl;39][aeg;38][aeh;37][acf;36][abf;36][cfg;36][bfg;36][cdg;36][bdg;36][cel;35][cfl;34][bce;33][ajl;33][adg;32][cgh;30][fhl;30][afh;30][ade;27][abj;26][dgl;26][e hl;25][ceh;25][afg;24][bdf;24][cdf;24][dfg;24][egl;23][ceg;22][beg;22][aef;22][fgl;21][c gj;20][cfh;20][agj;20][aej;20][acj;20][bgh;20][bgj;20][bjl;20][efl;17][del;17][dfl;17][adf; 16][bcj;13][adj;13][cjl;13][bdj;13][cdj;13][beh;12][efh;12][deh;12][egh;12][bde;11][deg; 11][cde;11][ejl;10][dgj;10][dgh;10][fgh;10][dfh;10][bfh;10][gjl;10][def;5][bef;5][efg;5][c ef;5].

The percent of host bacterial strains (as classified by MLST) that are infected by three phages of a phage combination are provided below. This trait is referred to as “at least 3 phage % coverage.” The phage combinations are ordered in descending performance grade. The combinations are:

[chl;40][abl;35][ahl;35][bcl;31][acl;31][bhl;30][abc;29][bdl;29][bch;28][ach;28][bcg;28][bcd;27][dhl;25][abd;24][cdh;23][abh;23][bdh;23][cdl;22][adl;20][acd;20][acg;20][abg;20][adh;19][ael;17][cgl;17][bgl;17][cdg;16][bdg;16][agl;13][ehl;12][aeh;12][cdf;12][bdf;12][bcf;12][bgh;10][cgh;10][afh;10][fgh;10][cfh;10][bfh;10][fhl;10][dgh;10][ghl;10][agh;10][dfh;10][dgl;8][bfl;8][dfl;8][afl;8][cfl;8][abf;8][adg;8][adf;8][acf;8][cel;5][efl;5][del;5][bel;5][bef;5][def;5][ade;5][bce;5][aef;5][cde;5][abe;5][bde;5][ace;5][cef;5][fgl;4][dfg;4][cfg;4][afg;4][bfg;4].

### 4 Phage Combinations

4 phage combinations were analyzed in silico for their ability to lyse the 85 different strains of Pseudomonas aeruginosa bacteria as classified by MLST, based on the ability of the single phage to lyse a particular MLST as assessed by a solid media assay.

The combinations and % of bacterial MLSTs are provided herein below. This trait is referred to as “at least 1 phage % coverage.” The number following each combination refers to the Percent trait performance—in this case the percent of MLSTs that are targeted by the phage combination. The combinations are listed in descending performance grade. The combinations are:

[acfl;100][ahjl;100][adjl;100][abgl;100][cehl;100][abfl;100][acel;100][aejl;100][a bel;100][afjl;100][abdl;100][agjl;100][ehjl;100][acdl;100][acjl;100][behl;100][abcl;100][acgl;100][abhl;100][afgl;95][adfl;95][adgl;95][adhl;95][achl;95][bchl;95][bdhl;95][adel;9 4][bdel;94][aefl;94][aegl;94][cefl;94][cdel;94][bcel;94][begl;94][befl;94][cegl;94][bcdl;9 3][abjl;93][abcd;90][bejl;90][cfhl;90][aghl;90][cghl;90][cejl;90][afhl;90][bfhl;90][egjl;90][cdhl;90][bghl;90][dejl;90][efgl;88][degl;88][abfg;88][abdg;88][abdf;88][abcg;88][abcf; 88][aehl;87][eghl;87][efhl;87][deh;87][cdg;86][bfg;86][cfg;86][cdfl;86][bcfl;86][bdfl; 86][bcgl;86][bdgl;86][abdj;86][abcj;86][bdjl;86][bcjl;86][abdh;85][abch;85][acdh;85][ab eg;83][abef;83][abde;83][abce;83][dfg;82][defl;82][bhj;80][bfjl;80][bgjl;80][acfg;80][c gjl;80][fghl;80][acgh;80][dgjl;80][efjl;80][dghl;80][dfjl;80][dfhl;80][acdg;80][acdf;80][a bej;80][fgjl;80][cfjl;80][abgj;80][abgh;80][abfj;80][abfh;80][acfh;80][cdjl;80][bcdh;76][a dfg;76][begh;75][befh;75][fhjl;75][ghjl;75][bceh;75][abeh;75][aceh;75][bdeh;75][acdj;73][aceg;72][bcef;72][bceg;72][bcde;72][acef;72][acde;72][afgh;70][adfh;70][adgh;70][bef g;66][bdeg;66][adef;66][adeg;66][aefg;66][bdef;66][cegh;62][aegh;62][aefh;62][cdeh;62][cefh;62][adeh;62][adfj;60][afgj;60][chjl;60][adgj;60][adhj;60][achj;60][acgj;60][aefj;60][dhjl;60][aegj;60][acfj;60][acej;60][abhj;60][adej;60][bdfh;60][bcgh;60][begj;60][bfgh;6 0][befj;60][bcfg;60][bcfh;60][bcdg;60][bdej;60][bcdf;60][bcej;60][bdgh;60][bdfg;56][cef g;55][cdeg;55][cdef;55][bcdj;53][cehj;50][eghj;50][afhj;50][aghj;50][degh;50][cdgh;50][cdfh;50][behj;50][efgh;50][aehj;50][cfgh;50][cdfg;48][bcgj;40][efgj;40][dfgh;40][bcfj;40][cegj;40][bfgj;40][bchj;40][degj;40][bdhj;40][cdej;40][bdgj;40][bdfj;40][cefj;40][defg;3 8][defh;37][defj;30][efhj;25][fghj;25][bfhj;25][dghj;25][dehj;25][bghj;25][cghj;25][cfhj; 25][dfgj;20][cfgj;20][cdfj;20][cdgj;20][cdhj;20].

The percent of host MLSTs that are infected by two phages of a phage combination are provided below. This trait is referred to as “at least 2 phage % coverage.” The phage combinations are ordered in descending performance grade. The combinations are:

[abcl;81][abdl;79][abel;76][acdl;75][bcdl;75][abhl;75][abfl;73][abch;71][bcel;70][adhl;70][achl;70][bdhl;70][bchl;70][abgl;69][abcd;68][abjl;66][abdh;66][acfl;65][acel;6 4][aegl;64][abeh;62][aehl;62][bcdh;61][abce;61][adfl;60][adgl;60][afgl;60][acgl;60][bcgl;60][bcfl;60][aghl;60][cdhl;60][afhl;60][adel;58][begl;58][acdh;57][aefl;52][bdel;52][bdg l;52][bfgl;52][abcf;52][abcg;52][bghl;50][bfhl;50][bceh;50][aegh;50][behl;50][abeg;50][acde;50][abgh;50][abde;50][abef;50][acef;50][aceg;50][abfh;50][aceh;50][bdfl;47][befl;4 7][cegl;47][adjl;46][bcdg;44][bcdf;44][abdg;44][bcfg;44][abfg;44][acdg;44][acdf;44][acf g;44][abdf;44][cfgl;43][cdgl;43][cdel;41][bcfh;40][abej;40][bcgh;40][agjl;40][acjl;40][dg hl;40][cfhl;40][adgh;40][afgh;40][aejl;40][bgjl;40][acgh;40][abhj;40][acej;40][cghl;40][f ghl;40][bejl;40][acfh;40][aegj;40][cdfl;39][aefg;38][adeg;38][cegh;37][cehl;37][aefh;37][eghl;37][adeh;37][adfg;36][bdfg;36][cdfg;36][cefl;35][bceg;33][abcj;33][bdjl;33][bcjl;33][acdj;33][bcde;33][bcef;33][dfgl;30][bfjl;30][dfhl;30][cdgh;30][cfgh;30][afjl;30][adfh;3 0][adej;30][degl;29][adef;27][cdjl;26][abdj;26][bcdj;26][cefh;25][cdeh;25][cghj;25][bghj:25][efhl;25][dehl;25][ghjl;25][aehj;25][begh;25][aghj;25][efgl;23][bdeg;22][cefg;22][be fg;22][cdeg;22][bcej;20][bchj;20][bcgj;20][bcfj;20][cegj;20][bfgh;20][ahjl;20][cgjl;20][c fgj;20][chjl;20][afgj;20][bfgj;20][cejl;20][adgj;20][aefj;20][abgj;20][cdgj;20][achj;20][ac gj;20][dgjl;20][cdfh;20][bdgh;20][bdgj;20][abfj;20][egjl;20][acfj;20][bhjl;20][begj;20][d efl;17][degh;12][befh;12][efgh;12][bdeh;12][defh;12][defg;11][bdef;11][cdef;11][bdfj;10][dfgh;10][fgjl;10][efjl;10][cdej;10][cdfj;10][bdfh;10][adfj;10][dfgj;10][cfjl;10][dejl;10][degj;10][bdej;10].

The percent of host bacterial strains (as classified by MLST) that are infected by three phages of a phage combination are provided below. This trait is referred to as “at least 3 phage % coverage.” The phage combinations are ordered in descending performance grade. The combinations are:

[abhl;55][achl;55][abcl;50][bchl;50][abdl;50][abch;47][adhl;45][cdhl;45][bcdl;43][acdh;42][acdl;41][abcd;40][aghl;40][acgl;39][abgl;39][abdh;38][bcdh;38][bcfg;36][abcg;36][bcdg;36][acel;35][abel;35][bdhl;35][bcgl;34][abdg;32][abcf;32][acdg;32][bcfl;30][af hl;30][acgh;30][cghl;30][abce;27][abfl;26][acfl;26][bdgl;26][adgl;26][cdgl;26][cehl;25][a ceh;25][aehl;25][acfg;24][cdfg;24][abfg;24][bdfg;24][bcdf;24][aegl;23][aceg;22][bceg;2 2][abeg;22][cfgl;21][bfgl;21][bcgj;20][cfhl;20][bghl;20][abgj;20][acgj;20][bcgh;20][acfh;20][abgh;20][adel;17][bcel;17][aefl;17][bdfl;17][afgl;17][cdfl;17][acdf;16][abdf;16][abd j;13][abcj;13][acjl;13][dfgl;13][behl;12][eghl;12][adeh;12][dehl;12][aegh;12][aefh;12][ef hl;12][abeh;12][adfg;12][begl;11][cegl;11][cdeg;11][adeg;11][abde;11][bcde;11][bdeg;1 1][acde;11][bdfh;10][cfgh;10][abfh;10][bdgh;10][bdgj;10][dghl;10][bgjl;10][fghl;10][bfh l;10][bfgh;10][cdfh;10][dfhl;10][aejl;10][cdgj;10][afgh;10][agjl;10][adgj;10][adgh;10][ad fh;10][cgjl;10][dfgh;10][cdgh;10][bcfh;10][adfl;8][bcdj;6][bcjl;6][acdj;6][abjl;6][befl;5][cefl;5][cdel;5][bdel;5][defl;5][degl;5][efgl;5][defg;5][cefg;5][cdef;5][bdef;5][bcef;5][aefg;5][abef;5][acef;5][adef;5][befg;5].

The percent of host bacterial MLSTs that are infected by four phages of a phage combination are provided below. This trait is referred to as “at least 4 phage % coverage.” The phage combinations are ordered in descending performance grade. The combinations are:

[bchl;30][achl;30][abcl;27][bdhl;25][abhl;25][cdhl;25][abch;23][bcdh;23][bcdl;22][abdl;20][abcd;20][abcg;20][adhl;20][acdh;19][abdh;19][acdl;18][bcgl;17][bcdg;16][abg l;13][acgl;13][aehl;12][bcdf;12][bdfh;10][bdgh;10][bcfh;10][bcgh;10][adfh;10][bghl;10][aghl;10][acgh;10][acfh;10][bfgh;10][afgh;10][adgh;10][bfhl;10][abfh;10][abgh;10][fghl; 10][dghl;10][dfhl;10][dfgh;10][cghl;10][cfhl;10][cfgh;10][afhl;10][cdgh;10][cdfh;10][cd gl;8][adfl;8][abfl;8][bdfl;8][bcfl;8][bdgl;8][acfl;8][cdfl;8][acdg;8][acdf;8][abdg;8][abdf;8 ][abcf;8][bcel;5][acel;5][aefl;5][befl;5][defl;5][abel;5][cdel;5][adel;5][bdel;5][cefl;5][bce f;5][abce;5][bcde;5][cdef;5][bdef;5][acef;5][adef;5][acde;5][abef;5][abde;5][afgl;4][dfgl; 4][adgl;4][cfgl;4][bfgl;4][bcfg;4][adfg;4][cdfg;4][bdfg;4][abfg;4][acfg;4].

### 5 Phage Combinations

5 phage combinations were analyzed in silico for their ability to lyse the 85 different strains of Pseudomonas aeruginosa bacteria as classified by MLST, based on the ability of the single phage to lyse a bacteria of a particular MLST as assessed by a solid media assay.

The combinations and % of bacterial MLSTs are provided herein below. This trait is referred to as “at least 1 phage % coverage.” The number following each combination refers to the Percent trait performance—in this case the percent of bacteria MLSTs that are targeted by the phage combination. The combinations are listed in descending performance grade. The combinations are:

[abefl;100][aefjl;100][acghl;100][abhjl;100][abgjl;100][abghl;100][dehjl;100][abf jl;100][abfhl;100][aghjl;100][abfgl;100][acgjl;100][achjl;100][abejl;100][abehl;100][befh l;100][abegl;100][acfjl;100][acfhl;100][acdel;100][acdjl;100][ceghl;100][acejl;100][acefl;100][cehjl;100][cefhl;100][aegjl;100][acdhl;100][afhjl;100][aehjl;100][acdgl;100][beghl; 100][acfgl;100][acdfl;100][afgjl;100][acehl;100][acegl;100][behjl;100][adejl;100][abcgl; 100][efhjl;100][abchl;100][adgjl;100][abcjl;100][abcdl;100][bdehl;100][bcehl;100][abdel;100][cdehl;100][abdfl;100][adhjl;100][abdgl;100][abcel;100][abdhl;100][eghjl;100][adfj l;100][abdjl;100][abcfl;100][adfgl;95][bcdhl;95][cefgl;94][bdefl;94][aefgl;94][bcegl;94][bdegl;94][cdefl;94][cdegl;94][adegl;94][adefl;94][befgl;94][bcdel;94][bcefl;94][adghl;90][cdfhl;90][adfhl;90][cdghl;90][cdejl;90][begjl;90][cefjl;90][bcghl;90][befjl;90][bcfhl;90][bcejl;90][efgjl;90][bdejl;90][bdfhl;90][cegjl;90][bdghl;90][degjl;90][bfghl;90][cfghl;90][afghl;90][defjl;90][defgl;88][abcdf;88][abcdg;88][abcfg;88][abdfg;88][adehl;87][defhl;8 7][efghl;87][aefhl;87][deghl;87][aeghl;87][bdfgl;86][bcdgl;86][bcfgl;86][bcdfl;86][cdfgl; 86][bcdjl;86][abcdj;86][abcdh;85][abcde;83][abdef;83][abcef;83][abefg;83][abceg;83][ab deg;83][bfgjl;80][abdgj;80][abdgh;80][abdfj;80][abdfh;80][abdej;80][abcgj;80][abefj;80][abcgh;80][abcfj;80][abcfh;80][abcej;80][bchjl;80][bcgjl;80][bdfjl;80][bcfjl;80][bdgjl;80][dfghl;80][cdfjl;80][cdgjl;80][acfgh;80][cfgjl;80][acdgh;80][acdfg;80][acdfh;80][abfgj;80][abfgh;80][dfgjl;80][abegj;80][bdhjl;80][bfhjl;75][bdegh;75][cghjl;75][cfhjl;75][bdefh;7 5][dfhjl;75][befgh;75][bghjl;75][dghjl;75][fghjl;75][acdeh;75][bcdeh;75][abceh;75][aceg h;75][abdeh;75][acefh;75][abefh;75][abegh;75][bcegh;75][bcefh;75][bcdeg;72][acefg;72][bcefg;72][bcdef;72][acdeg;72][acdef;72][adfgh;70][adefg;66][bdefg;66][adegh;62][cdef h;62][cdegh;62][adefh;62][aefgh;62][cefgh;62][adefj;60][acdhj;60][cdhjl;60][acfgj;60][a cegj;60][acefj;60][acdgj;60][acdfj;60][acdej;60][abdhj;60][abchj;60][adfgj;60][adegj;60][bcdfh;60][bcdej;60][bcfgh;60][bdefj;60][aefgj;60][bcegj;60][bdegj;60][bcefj;60][bdfgh;6 0][bcdgh;60][bcdfg;60][befgj;60][cdefg;55][abehj;50][ceghj;50][defgh;50][aeghj;50][deg hj;50][efghj;50][bcehj;50][adghj;50][abfhj;50][adfhj;50][cdfgh;50][acehj;50][cdehj;50][c efhj;50][acfhj;50][bdehj;50][aefhj;50][befhj;50][acghj;50][beghj;50][abghj;50][afghj;50][adehj;50][bdfgj;40][bcdhj;40][cdegj;40][defgj;40][bcdgj;40][cdefj;40][cefgj;40][bcfgj;40][bcdfj;40][bfghj;25][cdfhj;25][dfghj;25][defhj;25][bdfhj;25][bdghj;25][bcfhj;25][cfghj;2 5][cdghj;25][beghj;25][cdfgj;20].

The percent of host bacterial MLSTs that are infected by two phages of a phage combination are provided below. This trait is referred to as “at least 2 phage % coverage.” The phage combinations are ordered in descending performance grade. The combinations are:

[abcdl;87][abcel;82][abchl;80][abdhl;80][abcgl;78][abcfl;78][abdel;76][abefl;76][abegl;76][abcdh;76][abceh;75][bcdhl;75][abdgl;73][abfgl;73][abdfl;73][bcdel;70][bcefl;7 0][bcegl;70][abejl;70][acdhl;70][abdjl;66][abcjl;66][acfgl;65][acdgl;65][acdfl;65][adfgl;6 5][acegl;64][acefl;64][adegl;64][acdel;64][aefgl;64][aeghl;62][bcehl;62][abefh;62][abehl; 62][acehl;62][aefhl;62][abdeh;62][abegh;62][adehl;62][abcde;61][abcef;61][abceg;61][bc fgl;60][bcdgl;60][bcdfl;60][abgjl;60][bcfhl;60][acfhl;60][acghl;60][bcghl;60][abghl;60][a bfjl;60][abfhl;60][abcgh;60][adfhl;60][adghl;60][afghl;60][abcfh;60][adefl;58][bdegl;58][befgl;58][acdjl;53][bdefl;52][bdfgl;52][abcdf;52][abcfg;52][abcdg;52][abdfh;50][begjl;5 0][bcefh;50][adegh;50][bdfhl;50][bcdeh;50][adejl;50][bfghl;50][bcejl;50][bdghl;50][acde g;50][beghl;50][abfgh;50][aefgh;50][abefg;50][befhl;50][abehj;50][aehjl;50][aeghj;50][a cdef;50][acejl;50][acdeh;50][abdgh;50][bcegh;50][acefg;50][acefh;50][acegh;50][bdehl;5 0][abdeg;50][aegjl;50][acehj;50][abdef;50][bdejl;50][cdegl;47][cefgl;47][bcdjl;46][abdfg;44][acdfg;44][bcdfg;44][cdfgl;43][cdefl;41][bcfjl;40][bcdfh;40][bcdgh;40][bdfjl;40][beg jl;40][afgjl;40][abcdj;40][bcfgh;40][abcej;40][bdgjl;40][abegj;40][aefjl;40][acgjl;40][abd hj;40][cfghl;40][abhjl;40][acdej;40][acdfh;40][acdgh;40][cdghl;40][cdfhl;40][acefj;40][a bdej;40][acegj;40][acfjl;40][acfgh;40][abchj;40][adgjl;40][aefgj;40][adegj;40][abefj;40][bfgjl;40][dfghl;40][befjl;40][adfgh;40][adfjl;40][adefg;38][ceghl;37][cefhl;37][cdegh;37][cefgh;37][cdehl;37][adefh;37][efghl;37][deghl;37][bcdef;33][bcdeg;33][bcefg;33][cdejl; 30][adefj;30][degjl;30][cdfgh;30][cegjl;30][defgl;29][acfhj;25][acghj;25][bcfhj;25][cdefh;25][bfhjl;25][adehj;25][bfghj;25][behjl;25][bghjl;25][abghj;25][cdghj;25][beghj;25][abf hj;25][ceghi;25][cehjl;25][cfghj;25][cfhjl;25][cghjl;25][defhl;25][dghjl;25][eghjl;25][adg hj;25][fghjl;25][bdghj;25][befgh;25][aefhj;25][aghjl;25][bcehj;25][afhjl;25][afghj;25][be ghj;25][bdegh;25][cdefg;22][bdefg;22][bcdgj;20][cefgj;20][bdegj;20][acdfj;20][bcdhj;20][cefjl;20][abfgj;20][adhjl;20][bcefj;20][abdgj;20][cfgjl;20][cdgjl;20][bchjl;20][abdfj;20][bcegj;20][cdhjl;20][acdgj;20][abcgj;20][efgjl;20][befgj;20][adfgj;20][bdhjl;20][achjl;20][bdfgh;20][bcdej;20][bcfgj;20][cdegj;20][bcdfj;20][abcfj;20][cdfjl;20][acdhj;20][dfgjl;20][acfgj;20][cdfgj;20][bdfgj;20][bdefh;12][defgh;12][bdefj;10][cdefj;10][defjl;10][defgj;10].

The percent of host MLSTs that are infected by three phages of a phage combination are provided below. This trait is referred to as “at least 3 phage % coverage.” The phage combinations are ordered in descending performance grade. The combinations are:

[abchl;65][acdhl;60][abdhl;60][abcdl;58][bcdhl;55][abcel;52][abghl;50][abfhl;50][abehl;50][abcfl;47][abcdh;47][abegl;47][acegl;47][abcgl;43][abdel;41][acdel;41][abcfg; 40][acfhl;40][afghl;40][abcdg;40][acghl;40][abcdf;40][adghl;40][abdgl;39][abdfl;39][acf gl;39][abfgl;39][bcdgl;39][bcfgl;39][acdfl;39][acdgl;39][acegh;37][abceh;37][acehl;37][c eghl;37][bcehl;37][aeghl;37][bcdfg;36][acdfg;36][abdfg;36][abefl;35][acefl;35][bcdfl;34][cdfgl;30][bdfgl;30][adfhl;30][acfgh;30][abcfh;30][bcghl;30][acdgh;30][bcfhl;30][cdghl; 30][cfghl;30][abcgh;30][adegl;29][bcegl;29][abcde;27][abcef;27][abceg;27][adfgl;26][ad ehl;25][cdehl;25][cefhl;25][bghjl;25][aghjl;25][acefh;25][aefhl;25][acghj;25][bcghj;25][a begh;25][abghj;25][beghl;25][cghjl;25][acdeh;25][bcdel;23][aefgl;23][abdeg;22][abefg;2 2][acdeg;22][acefg;22][bcdeg;22][bcefg;22][bcfgh;20][bcgjl;20][bdghl;20][abcdj;20][bcd gj;20][bchjl;20][adgjl;20][bcdgh;20][bcegj;20][bcfgj;20][abcej;20][abcfj;20][abcgj;20][a bcjl;20][aegjl;20][abchj;20][bdgjl;20][abdgh;20][acegj;20][abejl;20][acejl;20][acfgj;20][a cdjl;20][abdjl;20][bfghl;20][abdgj;20][acgjl;20][abfgh;20][abfgj;20][achjl;20][acdgj;20][cdgjl;20][acdfh;20][abgjl;20][abhjl;20][cdfhl;20][abegj;20][bcefl;17][bdegl;17][adefl;17][cdegl;17][bcdjl;13][efghl;12][abdeh;12][aefgh;12][bcegh;12][adefh;12][deghl;12][befhl; 12][defhl;12][bdehl;12][adegh;12][abefh;12][befgl;11][cefgl;11][cdefg;11][bdefg;11][bc def;11][acdef;11][abdef;11][adefg;11][abdfj;10][adegj;10][bdfgh;10][bdfgj;10][adfgh;10][acdfj;10][bdfhl;10][cfgjl;10][adejl;10][cegjl;10][begjl;10][bdegj;10][acfjl;10][bfgjl;10][b cdfj;10][abfjl;10][cdegj;10][acdej;10][cdfgh;10][adfgj;10][cdfgj;10][bcdfh;10][abdfh;10][bcejl;10][afgjl;10][dfghl;10][aefjl;10][bcfjl;10][abdej;10][bcdej;10][bdefl;5][cdefl;5][def gl;5].

The percent of host bacterial MLST that are infected by four phages of a phage combination are provided below. This trait is referred to as “at least 4 phage % coverage.” The phage combinations are ordered in descending performance grade. The combinations are:

[abchl;45][acdhl;40][abcdh;38][abcdl;37][abdhl;35][bcdhl;35][abcgl;34][abcdg;3 2][acghl;30][bcdgl;26][abdgl;26][acdgl;26][acehl;25][bcdfg;24][abcfg;24][abceg;22][abc fl;21][bcfgl;21][acfhl;20][abcgj;20][bcghl;20][abghl;20][abcgh;20][abcel;17][abfgl;17][a cfgl;17][bcdfl;17][abcdf;16][cdfgl;13][bdfgl;13][aefhl;12][aeghl;12][adehl;12][abehl;12][acdfg;12][abdfg;12][abegl;11][bcegl;11][acegl;11][acdeg;11][abcde;11][bcdeg;11][abdeg;11][acfgh;10][cdghl;10][acdfh;10][cdfgh;10][acdgh;10][acdgj;10][cdfhl;10][bcdgj;10][af ghl;10][bcdgh;10][abcfh;10][cfghl;10][bcdfh;10][adfgh;10][dfghl;10][bdfgh;10][bdfhl;10][abdfh;10][bcfgh;10][acgj;10][bdghl;10][abfgh;10][bfghl;10][adghl;10][abfhl;10][bcgjl; 10][abdgj;10][abdgh;10][abgjl;10][bcfhl;10][adfhl;10][adfgl;8][acdfl;8][abdfl;8][abcjl;6][abcdj;6][aefgl;5][adefl;5][adegl;5][bcdel;5][acefl;5][bcefl;5][acdel;5][bdefl;5][bdegl;5][a befl;5][befgl;5][cdefl;5][cdegl;5][abdel;5][cefgl;5][defgl;5][bcefg;5][cdefg;5][bdefg;5][b cdef;5][abcef;5][acefg;5][abdef;5][abefg;5][acdef;5][adefg;5].

### Example 4 Synergism of Phages and Antibiotics

### Materials and Methods

Synergism with Antibiotic in Liquid Infection

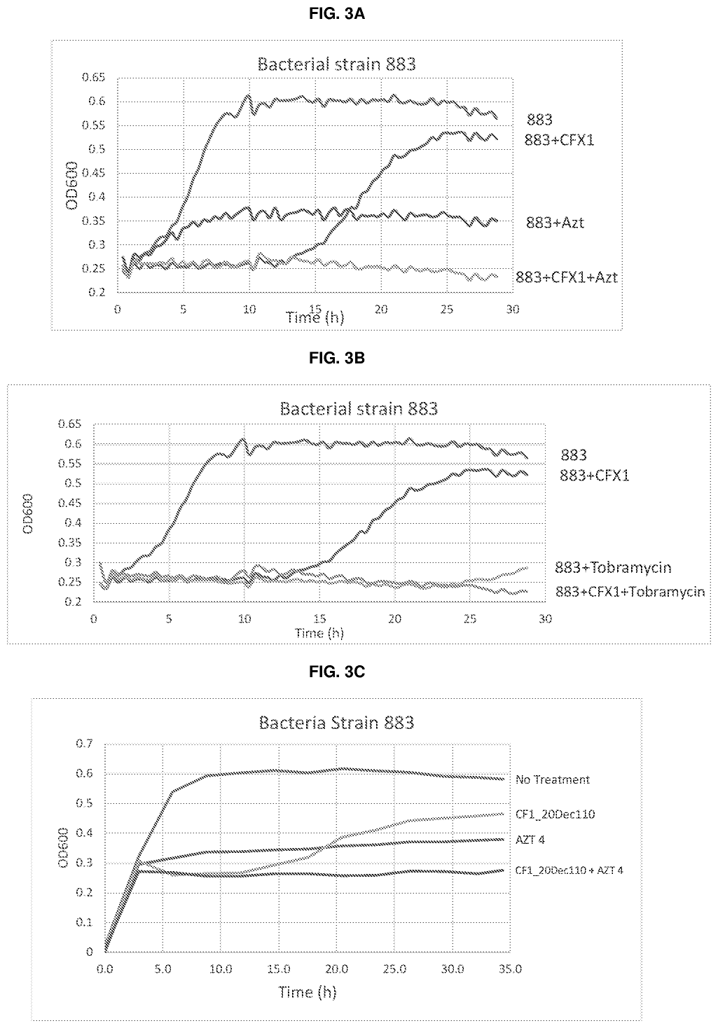

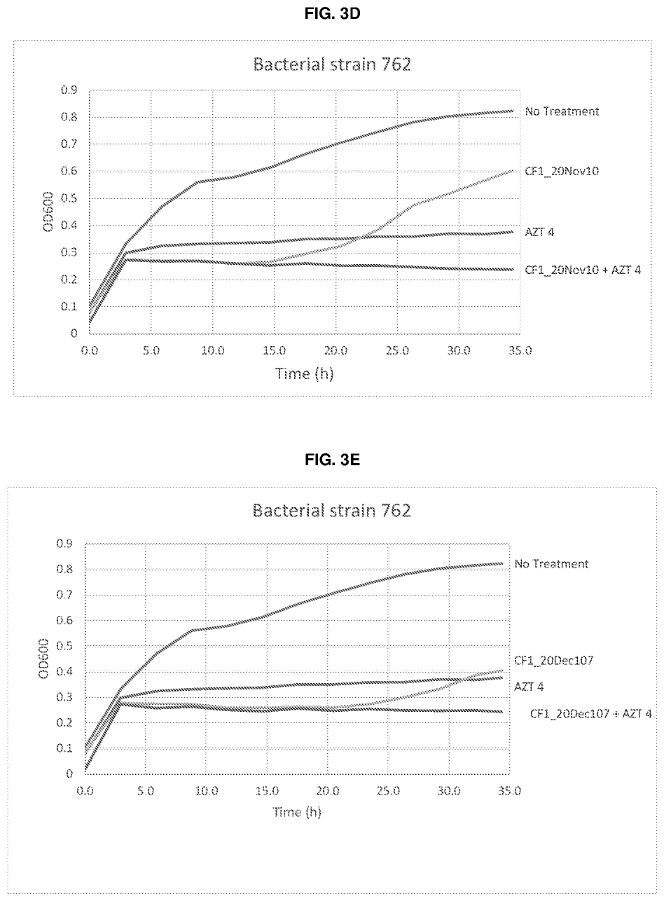

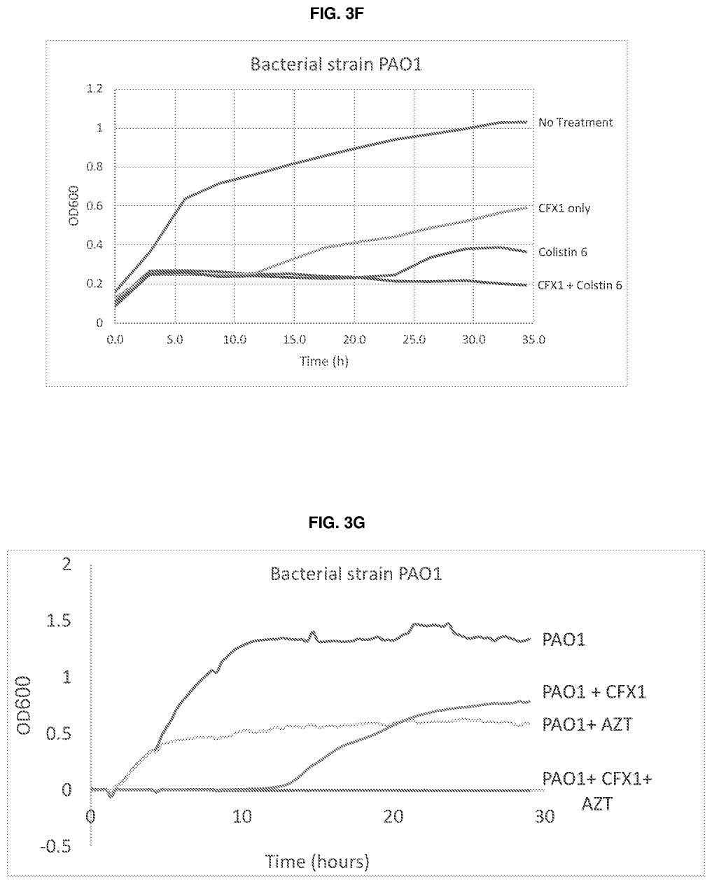

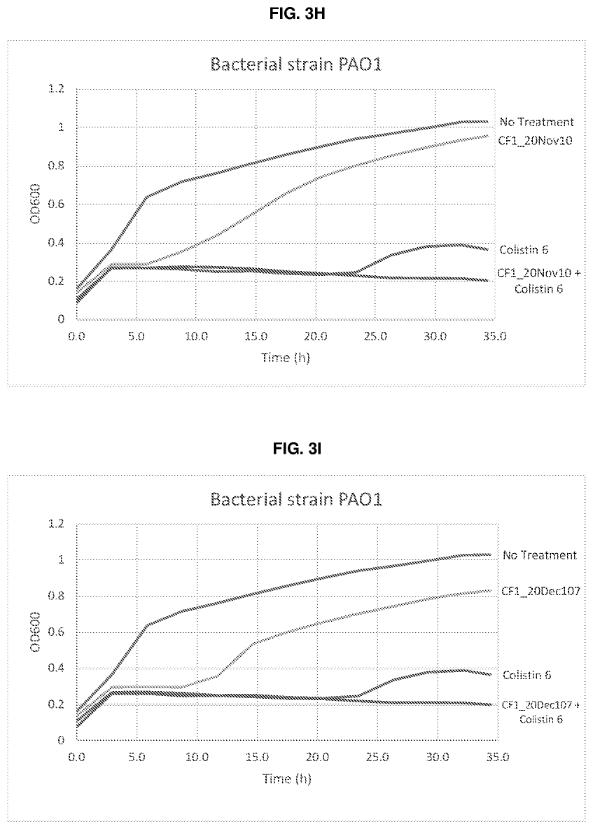

To test the efficacy of the phage and antibiotics in liquid culture, bacterial host cells were grown overnight in TSB at 37° C. with shaking at 180 rpm to OD600>1.5. In addition, each phage was diluted to a concentration of 5×10\hat{ }7 PFU/ml in TSB and used individually or combined equally with other phages into a cocktail. Then, 1 mM ions were added, and 200 μL was dispensed per well in a 96-well plate to a final concentration of 10∂PFU/well. For no phage control (NPC), TSB containing 1 mM ions was used. Antibiotics was added to the relevant wells (aztreonam 4 μg/mL, colistin 2 μg/mL for “883,” 4 μg/mL for “762” and 6 μg/mL for PAO1). Then, 2 μL of the bacterial culture was added to the wells (dilution of 1:100). TSB containing 1 mM ions was used as NPC. Two repeats were done for each treatment, and TSB media served as a blank. 50 μL of mineral oil added were added to each well to reduce evaporation of the samples, and the plate was covered with a sterile film to allow for bacterial growth and keep the culture sterile. Plates were incubated for approximately 30 hours in a plate reader at 37° C. with shaking, and OD600 was measured every 20 minutes. Two biological repeats were performed for the assay. The results are set forth in FIGS. 3A-3J.

FIG. 3A presents the growth curves expressed by the OD600 measures for (1) bacterial strain “883” with no treatment, (2) “883” treated with cocktail CFX1, (3) “883” treated with aztreonam, and (4) “883” treated with cocktail CFX1 and aztreonam (Azt). FIG. 3A demonstrates a synergistic reduction of bacterial growth achieved by the cocktail and aztreonam. This effect was also measured with additional bacterial strains.

FIG. 3B presents the growth curves expressed by the OD600 measures for (1) bacterial strain “883” with no treatment, (2) “883” treated with cocktail CFX1, (3) “883” treated with tobramycin, and (4) “883” treated with cocktail CFX1 and tobramycin. FIG. 3B demonstrates a synergistic reduction of bacterial growth achieved by the cocktail and tobramycin. This effect was also measured with additional bacterial strains.

FIG. 3C presents the growth curves expressed by the OD600 measures for (1) bacterial strain “883” with no treatment, (2) “883” treated with phage CF1_20Dec110, (3) “883” treated with aztreonam, and (4) “883” treated with phage CF1_20Dec110 and aztreonam. FIG. 3C demonstrates a synergistic reduction of bacterial growth achieved by CF1_20Dec110 and aztreonam. This effect was also measured with additional bacterial strains.

FIG. 3D presents the growth curves expressed by the OD600 measures for (1) bacterial strain “762” with no treatment, (2) “762” treated with phage CF1_20Nov10, (3) “762” treated with aztreonam, and (4) “762” treated with phage CF1_20Nov10 and aztreonam. FIG. 3D demonstrates a synergistic reduction of bacterial growth achieved by CF1_20Nov10 and aztreonam. This effect was also measured with additional bacterial strains.

FIG. 3E presents the growth curves expressed by the OD600 measures for (1) bacterial strain “762” with no treatment, (2) “762” treated with phage CF1_20Dec107, (3) “762” treated with aztreonam, and (4) “762” treated with phage CF1_20Dec107 and aztreonam. FIG. 3E demonstrates a synergistic reduction of bacterial growth achieved by CF1_20Dec107 and aztreonam. This effect was also measured with additional bacterial strains.

FIG. 3F presents the growth curves expressed by the OD600 measures for (1) bacterial strain “PAO1” with no treatment, (2) “PAO1” treated with cocktail CFX1, (3) “PAO1” treated with colistin, and (4) “PAO1” treated with cocktail CFX1 and colistin. FIG. 3F demonstrates a synergistic reduction of bacterial growth achieved by the cocktail and colistin. This effect was also measured with additional bacterial strains.

FIG. 3G presents the growth curves expressed by the OD600 measures for (1) bacterial strain “PAO1” with no treatment, (2) “PAO1” treated with cocktail CFX1, (3) “PAO1” treated with aztreonam, and (4) “PAO1” treated with cocktail CFX1 and aztreonam. FIG. 3G demonstrates a synergistic reduction of bacterial growth achieved by the cocktail and aztreonam. This effect was also measured with additional bacterial strains.

FIG. 3H presents the growth curves expressed by the OD600 measures for (1) bacterial strain “PAO1” with no treatment, (2) “PAO1” treated with phage CF1_20Nov10, (3) “PAO1” treated with colistin, and (4) “PAO1” treated with phage CF1_20Nov10 and colistin. FIG. 3H demonstrates a synergistic reduction of bacterial growth achieved by CF1_20Nov10 and colistin. This effect was also measured with additional bacterial strains.

FIG. 3I presents the growth curves expressed by the OD600 measures for (1) bacterial strain “PAO1” with no treatment, (2) “PAO1” treated with phage CF1_20Dec107, (3) “PAO1” treated with colistin, and (4) “PAO1” treated with phage CF1_20Dec107 and colistin. FIG. 3I demonstrates a synergistic reduction of bacterial growth achieved by CF1_20Dec107 and colistin. This effect was also measured with additional bacterial strains.

FIG. 3J presents the growth curves expressed by the OD600 measures for (1) bacterial strain “PAO1” with no treatment, (2) “PAO1” treated with phage CF1_20Dec110, (3) “PAO1” treated with colistin, and (4) “PAO1” treated with phage CF1_20Dec110 and colistin. FIG. 3J demonstrates a synergistic reduction of bacterial growth achieved by CF1_20Dec110 and colistin. This effect was also measured with additional bacterial strains.

In summary, exposure of the PsA strains to the phage cocktail CFX1 alone suppressed growth for 13-15 hours while exposure to aztreonam alone causes slower bacterial growth throughout the observed period. When bacterial strains (e.g., PAO1, 883) are exposed to CFX1 and aztreonam together the appearance of bacteria mutants was essentially obliviated (this synergism effect was also verified with other clinical isolates). In the case of Tobramycin, exposure of the PsA strains to the phage cocktail CFX1 alone suppressed growth for 13-15 hours, (like with aztreonam) while exposure to Tobramycin alone suppressed growth for approximately 25 hours. When bacterial strains (e.g., PAO1, 883) are exposed to CFX1 and aztreonam together the appearance of bacteria mutants was essentially obliviated (this synergism effect was also verified with other clinical isolates). A similar synergic effect of CFX1 and antibiotics was observed in the case of colistin. The synergistic effect was also observed for some of the individual phage members of CFX1 when combined with the various antibiotics and therefore is also expected in the case of Azithromycin and other antibiotics used against lung infections in general and with CF patients in particular.

### Example 5 Efficacy in Biofilm

### Materials and Methods: Growing Biofilm and Treatment

To test the effect of phage on biofilm, starters of the relevant bacteria were grown overnight. Three isolated single colonies of the relevant strains were picked into 3 mL TSB and shook overnight at 37° C., 180 rpm, to an OD₆₀₀>1, one starter used for each of two biological repeats. The next day, the starters were dispersed in 96 wells plate and the biofilm was left to form for 24 hours: cultures were diluted 1:100 in 3 mL fresh TSB (Tryptic Soy Broth) medium and OD₆₀₀ was adjusted to ˜0.05 (˜4×10⁷ CFU/mL). 200 μL of the diluted inoculums were added per well in a 96 well culture plate (Biofil, cat #TCP-011-096) according to the plate layout. 200 μL TSB was used for blank wells. The microtiter plates were incubated at 37° C. for 24 hours, with low shaking frequency of 110 rpm. Then the supernatant (containing media, waste, and planktonic cells) was carefully removed, and wells, with the P. aeruginosa biofilm, were washed once with 200 μL PBS (Phosphate-buffered saline, Hylabs, cat #BP655). Then, the desired treatments were added and the 96 well plate was returned to the 37° C. shaker (110 rpm) for 6 hours. 200 μL of fresh media/treatment was added according to the plate layout. Treatment was either with 2.5×10⁷ PFU/well 3-phage cocktail CFX1 (as detailed in table 1) (at 1:1:1 ration) or the antibiotic imipenem (SIGMAC cat #C3809-1G) at 5- or 50-fold the minimal inhibitory concentration (MIC) of P. aeruginosa (4 μg/mL). TSB medium served as no treatment control. After the incubation period, the supernatant was carefully removed (media, waste, phages/antibiotics, and planktonic cells) the biofilm was washed again with 200 μL PBS (Phosphate-buffered saline, Hylabs, cat #BP655) and its biomass measured (staining with crystal violet (CV) and/or viable cells (plating for CFU enumeration).

### Assessment of the Phage Effect on Pre-Formed P. aeruginosa Biofilm Biomass by Crystal Violet Staining Assay

### Materials and Methods: Crystal Violet (CV) Staining Assay

For biomass assessment, crystal violet (CV), that stains DNA, live/dead bacterial cells, and the extracellular matrix was used.

Following the last step in the above-described procedure (“growing Biofilm and treatment”), 200 μL 0.1% crystal violet (Acros, cat #447570500) was added to each well of the microtiter plate. Following incubation at room temperature for 10-15 minutes, the plate was rinsed 3 three times with PBS. The microtiter plate was placed in the hood, upside down to dry for few hours or overnight. 200 μL of 70% ethanol was added to each well of the microtiter plate to solubilize the CV and the solution was transferred to a new flat bottomed microtiter dish. 70% ethanol was used as the blank. Plates were photographed and biofilm biomass quantification was done by measuring the absorbance of the CV stain in a plate reader, at OD₆₁₅. CV staining assay was performed in two biological repeats each with 6 technical repeats.

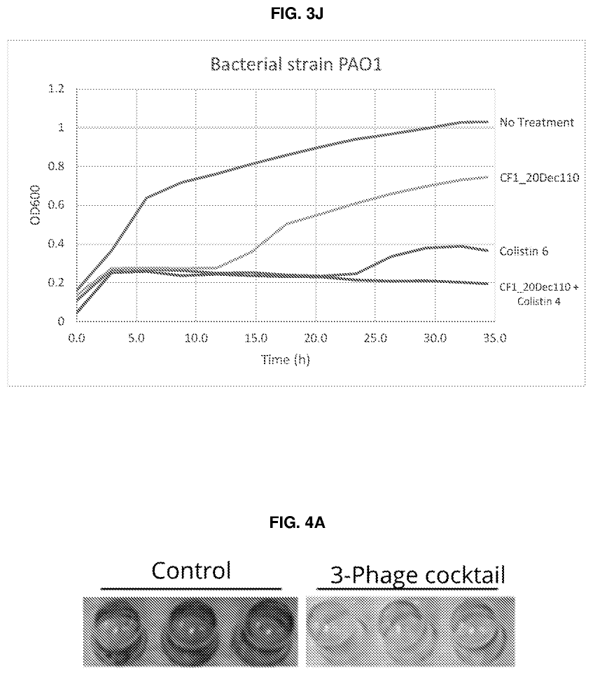

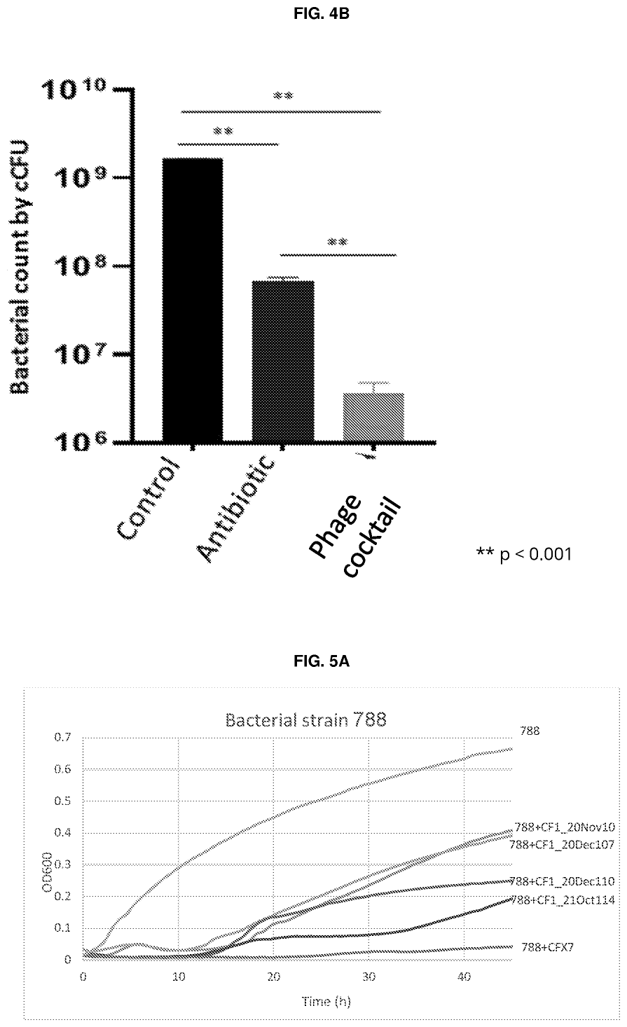

To assess the biofilm penetration, bacteria were grown in conditions allowing biofilm formation, and 24 h later CFX1 (as detailed in table 1) phage cocktail sample was added for 6 hours. Then, the biofilm mass was stained with crystal violet (FIG. 4A). Statistical analysis was performed using two-way ANOVA test for detection of significant differences between treatments. The biofilm's biomass produced by P. aeruginosa can be clearly visualized by the relatively dark purple color in the leftmost three wells on FIG. 4A, with no treatment, following CV staining. After phage cocktail treatment, a clear reduction in the pre-formed biofilm biomass was evident by the lighter purple color of the three wells on the right. These results indicated that phage cocktail was able to reduce the biofilm biomass.

### Assessment of Phage Effect on Viability of P. aeruginosa Embedded in Pre-Formed Biofilm

### Materials and Methods—Viability of P. aeruginosa in Pre-Formed Biofilm

For host cells viability assessment, the BacTiter-Glo (Promega, cat #G8231) method was used. The method measures viable bacterial cells based on quantification of the ATP present in the viable cells. The readout is a luminescent signal (in relative light units (RLU)) in an ATP-dependent reaction.

First, a calibration curve was established, to correlate the BacTiter-Glo assay results expressed in RLU with the viability assay results that is expressed in colony forming unites (CFU). A calibration curve, specific to P. aeruginosa, was established. This part was done on a planktonic culture: overnight culture in TSB was normalized to OD₆₀₀ of 1.3 and a series of ten-fold serial dilutions were prepared (10⁰ to 10⁻⁵). 200 μL from each dilution was dispersed per well, in a 96 well plate. The 96 well plate was centrifuged at 2272×g, 10 min at 4° C. and the pellet was resuspended in 100 μL PBS. 100 μL of BacTiter-Glo Reagent was added to each well of the 96-well plate and mixed well. Following incubation for 5 minutes at room temperature, RLU was measured in the luminometer. In parallel, 5 μL of the ten-fold serial dilutions were spotted on BHIS plates for CFU enumeration. Plotting RLU vs CFU showed good correlation (R²=0.962) from 1×10⁹ to 1×10⁵ RLU. The trendline equation was used in the experiments to convert RLU to calculated CFU (cCFU).

Following the last step in the above-described procedure (“growing Biofilm and treatment”), the plate was left to dry for 15 min at room temperature. 100 μL PBS+100 μL of BacTiter-Glo Reagent were added to each well of the 96 white well plates and incubated at RT. Following 5 minutes incubation, with low shaking frequency of 110 rpm, the lid was removed, and the plate was placed in a spark device (BiomX ID #186). Luminescent signal was measured using 1000 milliseconds integration time, in relative light units (RLU). Using the calibration curve equation, RLU were converted to cCFU.

The results are set forth in FIG. 4B. The results reveal that the CFX1 phages were able to penetrate biofilm and reduce bacterial burden of embedded PsA (˜2.5 logs), this reduction was greater than that obtained by antibiotic treatment of biofilm produced by an antibiotic sensitive PsA strain (˜1 log). The significant reduction by phage is also corroborated as a visible decrease in biofilm by staining with crystal violet, which stains DNA of dead bacterial cells and the extracellular matrix.

### Example 6 Phage Viability Following Prolong Storage

### Materials and Methods: Stability Assay

Phage viability following storage in different condition was measured by testing potency of each one of the phages at different temperature conditions (5° C., 25° C., 37° C.), time periods (1, 2, 4 and 8 weeks) and the following compositions:

| Buffer No. | Buffer composition |
|---|---|
| 1 | 10 mM Tris, 135 mM NaCl, 20 mM MgSO4, |
| | 0.02% Tween-80, pH 7.4 ± 0.2 |
| 2 | 10 mM Tris, 150 mM NaCl, 0.02% Tween-80, |
| | pH 7.4 ± 0.2 |
| 3 | 10 mM Tris, 155 mM NaCl, pH 7.4 ± 0.2 |

Phage titers for each experimental block were determined by spot drop plaque assay as follows: host culture was prepared by inoculating 4 mL liquid BHIS with 5-10 colonies of the host and incubating at 37° C., until OD600 was 1.5 (overnight). 150 p L of host culture were added to 4 mL of molten top agar (BHIS top agar: BHIS media, 0.4% Agarose) with divalent ions Mn²⁺, Ca²⁺ and Mg²⁺ and dispensed on BHIS agar plats (1.5% Agarose). Plate were left to solidify for 15 min at RT. Then dilutions of phage sample were dropped (5 μL). Plates were incubated overnight before counting plaques (10-50 plaques/drop) and determination of phage titer (number of plaques×200×reciprocal of counted dilution=PFU/mL). Phage titer at each time period was compared to titer at zero time and the corresponding log difference calculated. Results are presented in table 4 below.

TABLE 4

| | Temperature, | time, | Log difference from time 0 * | | |
|---|---|---|---|---|---|
| Phage | ° C. | weeks | Buffer 1 | Buffer 2 | Buffer 3 |
| CF1_20DEC107 | 5 | 2 | 0.19 | 0.19 | −0.42 |
| | | 4 | 0.16 | 0.35 | −0.07 |
| | 25 | 2 | −0.02 | 0.16 | −0.46 |
| | | 4 | 0.39 | 0.34 | −0.13 |
| | 37 | 1 | −0.10 | 0.10 | −0.16 |
| | | 2 | −0.81 | 0.21 | 0.35 |
| | | 4 | −0.52 | 0.60 | −0.79 |
| CF1_20NOV10 | 5 | 1 | −0.38 | −0.40 | NA |
| | | 2 | −0.19 | −0.25 | |
| | | 4 | −0.01 | −0.26 | |
| | | 8 | −0.22 | −0.39 | |
| | 25 | 1 | −0.38 | −0.61 | |
| | | 2 | −0.54 | −0.30 | |
| | | 4 | −0.22 | −0.39 | |
| | 37 | 1 | −0.59 | −0.61 | |
| | | 2 | −1.07 | −0.26 | |
| | | 4 | −0.89 | −0.51 | |
| CF1_20Dec110 | 5 | 1 | −0.08 | −0.35 | −2.02 |
| | | 2 | 0.03 | −0.48 | −2.03 |
| | 25 | 1 | −0.03 | −0.10 | −0.71 |
| | | 2 | −0.17 | −0.05 | −0.21 |
| | 37 | 1 | −0.12 | −0.58 | −0.68 |
| | | 2 | −0.20 | −0.63 | −1.51 |

* The average potency at time zero of each phage was 7.43E+09 PFU/ml.

### Example 7

### Essential Genes for the Phage Lytic Cycle

### Materials and Methods: Gene Analysis

According to certain embodiments, the phages' genomes are reduced in order to create synthetic phages with smaller genomes without a significant hamper of their essential functionality (e.g. the ability to infect and lyse a host bacteria). According to certain embodiments, such a reduced genome can then more readily accommodate a heterogenous molecule of DNA that otherwise, if added to the original full genomic DNA may be challenging due to the limited DNA encapsulation capacity of a phage (see for example Pires, D. P., Monteiro, R., Mil-Homens, D. et al. Designing P. aeruginosa synthetic phages with reduced genomes. Sci Rep 11, 2164 (2021). doi(dot)org/10.1038/s41598-021-81580-2). Additionally, or alternatively, the genetic sequences of the selected phages can be modified or optimized, e.g. for expression in a suitable producer cell line, provided the essential genes are relatively conserved.

According to certain embodiments, the following exemplary method for finding the likely essential genes is used. A gene X was defined as essential if it was recognized and assigned a function by PATRIC (docs(dot)patricbrc(dot)org/). In addition, if the gene's function is unknown by PATRIC (e.g. “hypothetical protein” or “phage protein”) the following test is performed: given a phage genome, for a gene X, count the number of homologs (global amino acid similarity of 30% or more using blastp) in all publicly available phage genomes infecting the same species (num.homologs(gene X)). Subsequently, the mean and standard deviation of number of homologs for each gene in the genomes that were found to contain gene X were computed, and the z-score for gene X was calculated as follows:

$$z ( gene X ) = \frac{\begin{matrix}[ num . homologs ( gene X ) ] - \\ \begin{matrix}[ mean num . homologs ( each gene in \\ each genome containing gene ) ]\end{matrix}\end{matrix}}{\begin{matrix}standard deviation num . \\ \begin{matrix}homologs ( each gene in each \\ genome containing gene X )\end{matrix}\end{matrix}}$$

Genes with z-scores over −1 were defined essential. All other genes were defined non-essential.

Below, is a list of essential genes for each phage. Each gene is represented by square brackets, containing the following data fields separated by semi-columns: first, the gene's start coordinate, end coordinate, and strand to relate to with relation to the phage genome sequence as presented in the sequence listing (+ is the strand given in the sequence listing). Second, the gene's function. (“HP” denotes a hypothetical protein and “PP” denotes an unclassified phage protein).

Essential genes of phage CF1_20Aug470: [1:438:−;PP][536:829:−;PP][829:1086:−;PP][1086:1412:−;PP][1409:1735:−;PP][1732:2151:−;PP][2227:2556:−;PP][2564:2764:−;PP][2780:4831:−;Phage DNA helicase][4821:5153:−;PP][5140:6168:−;PP][6332:7015:−;PP][7584:8309:−;PP][8360:8557:−;PP][8582:8896:−;PP][8886:9095:−:PP][9476:10060:+;PP][10177:11787:+;Phage terminase %2C large subunit][11777:13777:+;PP][13752:14321:+;PP][14497:14865:+;PP][14871:15617:+;PP][15632:16888:+;PP][16974:17390:+;PP][17393:18046:+;PP][18058:18405:+;PP][18685: 19035:+;PP][19036:19422:+;PP][19527:19898:+;PP][19909:23004:+;PP][23015:23932:+;Streptococcal hemagglutinin protein][23946:25679:+;PP][25697:31246:+;PP][31302:31583:−;PP][32232:32486:−;PP][32465:33355:−;PP][34530:36935:−;DNA polymerase I (EC 2.7.7.7)][37050:37379:−;PP][37453:37968:−;PP][37996:38952:−;PP][39043:39300:−;PP][39297:39764:−;PP][39757:39993:−;PP][39996:40670:−;PP][40667:41692:−;PP][41694:42005:−;PP][42002:42187:−;PP][42201:42899:−;PP][42912:43214:−;PP][43218:43343:−;PP].

Essential genes of phage CF1_20sep416: [37:183:−;HP][258:2003:−;Ribonucleotide reductase of class Ia (aerobic) % 2C alpha subunit (EC 1.17.4.1)][1996:3132:−;HP][3059:3403:−;HP][3405:4352:−;putative thymidylate synthase][4407:4499:−;HP][4559:5509:−;HP][5502:5669:−;HP][5718:6053:−;HP][6072:6287:−;HP][6299:6481:−;HP][6478:7257:−;HP][7254:7424:−;HP][7421:7858:−;HP][7855:8061:−;HP][8082:8468:−;HP][8465:9028:−;HP][9025:10077:−;HP][10119:10340:−;HP][10350:10583:−;HP][10646:11650:−;HP][11752:12468:−;HP][12470:12637:−;HP][12667:13065:−;HP][13155:13844:−;HP][14132:16132:−;HP][16193:18055:−;HP][18109:18294:−;HP][18291:18536:−;HP][18546:18737:−;HP][18724:18852:−;HP][18970:19392:−;HP][19393:19695:−;HP][19698:19862:−;HP][19849:20514:−;HP][21190:21591:−;HP][21678:21875:−;HP][22599:22832:+;HP][22862:24007:+;PP][24020:24178:+;HP][24171:24980:+;Phage protein (ACLAME 992)][25006:25326:+;HP][25338:25643:+;HP][25687:26001:−;HP][26037:26342:−;HP][26474:26917:−;HP][26904:27143:−;HP][27161:27721:−;Phage endolysin][27738:29237:−;HP][29251:29625:−;HP][29669:31726:−;HP][31737:32468:−;HP][32487:33950:−;HP][34334:35074:−;HP][35071:35988:−;HP][35985:36341:−;HP][36347:37108:−;HP][37105:39471:−;tail length tape-measure protein][39468:39620:−;HP][39728:40099:−;HP][40113:40592:−;HP][40592:40963:−;HP][41167:41691:−;HP][41722:43008:−;HP][43021:43584:−;HP][43581:43961:−;HP][43961:44233:−;HP][44412:44888:−;HP][44939:45973:−;HP][46018:46428:−;HP][46456:47373:−;HP][47370:47840:−;HP][47850:49289:−;HP][49302:50822:−;HP][53455:53778:−;HP][54582:55049:+;HP][55046:55402:+;HP][55450:55998:+;HP][56059:56244:+;HP][56241:56534:+;HP][56535:56720:+;HP][56755:57033:+;HP][57030:57308:+;HP][57322:57639:+;HP][57648:57917:+;HP][57914:58126:+;HP][58136:58372:+;HP][58402:5879 1:+;HP][58788:59996:+;HP][60009:60470:+;HP][60535:61095:+;HP][61097:61657:+;H P][61647:62078:+;HP][62078:62632:+;HP][62645:62881:+;HP][62883:63158:+;HP][63 155:63562:+;HP][63574:64491:+;HP][64502:64918:+;HP][64928:65803:+;putative nicotinamide phosphoribosyl transferase][65800:66018:+;HP][66075:67763: +;Nicotinamide phosphoribosyltransferase (EC2.4.2.12)][67774:67971:+;HP][67968:68447:+;HP][68459:68608:+;HP][68610:68921:+;HP][68918:69499:+;HP][6988 3:70386:+;HP][70388:70729:+;HP][70726:70941:+;HP][70997:71284:+;HP][71281:716 43:+;HP][71643:71954:+;HP][71942:72220:+;HP][72223:72909:+;HP][72933:73349:+; HP][73339:73749:+;HP][73742:74008:+;HP][74727:75320:−;HP][76114:76272:−;HP][76435:76605:−;HP][76655:76849:−;HP][76949:77215:−;HP][77310:77600:−;HP][77618:78070:−;HP][78605:79144:−;HP][79219:79734:−;HP][79818:80315:−;HP][80352:80666:−;HP][80678:80923:−;HP][81027:81419:−;HP][81416:81682:−;HP][81715:81939:−;HP][81958:82152:−;HP][82416:82769:−;HP][82769:83107:−;HP][83112:83783:−;HP][83859:84242:−;HP][84479:84625:−;HP][84699:85007:−;HP][85007:85135:−;HP][85206:85454:−;HP][85451:85738:−;HP][85741:85881:−;HP][86252:86725:−;HP][87354:87527:−;HP][87750:88736:−;HP][89176:89406:−;HP][89408:89602:−;HP][89612:90097:−;HP][90109:90357:−;HP][90398:90583:−;HP][90573:90887:−;HP][90887:91123:−;HP][91123:91356:−;HP].

Essential genes of phage CF1_20Dec107: [1:129:+;Phage-associated DNA primase][1091:1672:+;HP][1829:2443:−;HP][2636:3262:−;HP][3273:3584:−;HP][3637:3867:−;HP][3923:4147:−;HP][4209:4538:−;HP][4539:5183:−;HP][5215:5424:−;HP][5830:6081:−;HP][6761:6949:−;HP][6952:7674:−;Phage capsid and scaffold][7691:7993:−;HP][8004:8150:−;HP][8326:9708:+;Phage terminase %2C large subunit][9745:10128:−;HP][10128:10343:−;HP][10343:10693:−;HP][10737:11120:−;HP][11123:11902:−;HP][13040:13135:−;HP][13132:14064:−;PP][14168:14515:−;HP][14764:15075:−;HP][15081:15284:−;HP][15281:15604:−;HP][15636:16037:−;PP][16218:18515:+;PP][18515:19351:+;Phage minor capsid protein][19370:19576:+;PP][19573:19713:+;HP][20226:21659:+;PP][21663:22298:+;PP][22308: 23456:+;Phage capsid and scaffold][23558:23995:+;PP][24010:24477:+;PP][24501:24872:+;PP][24880:25431:+;PP][25428:26009:+;PP][26112:27539:+;PP][27598: 28050:+;Phage tail fiber][28050:28373:+;PP][28370:28720:+;PP][28722:29153:+;HP][29163:29666:+;PP][29666:30205:+;PP][30214:30807:+;Phage tail fiber][30817:31245:+;HP][31249:33825:+;Phage internal (core) protein][33825:34688:+;PP][34688:35221:+;PP][35277:35942:+;Phage baseplate][35999:37252:+;PP][37249:38763:+;PP][38768:41656:+;Phage tail fiber][41658:42086:+;HP][42086:42748:+;Phage endolysin][42773:43024:−;HP][43304:44215:−;DNA ligase %2C phage-associated][44270:44824:−;Phage DNA-binding protein][44821:45426:−;PP][45483:46379:−;PP][46468:47088:−;PP][47183:48742:−;Phage DNA helicase][48739:49149:−;PP][49142:52249:−;DNA polymerase III alpha subunit (EC 2.7.7.7)][55485:55703:−;Phage tail assembly protein][55687:55905:−;HP][55905:56135:−;PP][56223:57224:−;HP][57329:58219:−;HP][58380:59567:−;Phage DNA helicase][59554:59976:−;HP][60145:60930:+;HP][62309:62758:+;HP][62755:63831:+;HP][63837:64022:+;HP][64170:65780:+;Phage-associated DNA primase].

Essential genes of phage CF1_20Nov10: [1:1443:+;PP][1440:2129:+;PP][2126:2560:+;PP][2541:3482:+;PP][3482:3862:+;PP][3867:5381:+;PP][5532:8699:+;PP][8711:9598:+;PP][9613:9969:+;PP][10176:10382:−;PP][10369:10578:−;HP][10582:10800:−;PP][10797:11558:−;PP][11744:12730:−;PP][12705:13589:−;Phage exonuclease][13589:13873:−;PP][13845:14372:−;PP][14326:14880:−;PP][14950:16587:−;DNA polymerase (EC 2.7.7.7) % 2C phage-associated][16790:17299:−;PP][17376:17654:−;PP][17968:18201:−;HP][18237:18548:−;HP][18515:19024:−;DNA polymerase %2C phage-associated][19008:20717:−;Phage DNA primase/helicase][20718:21095:−;PP][21095:21493:−;PP][21493:22374:−;PP][22505:22726:−;PP][22736:24268:−;PP][24280:25455:−;PP][25431:26000:−;PP][26790:27746:−;PP][27765:28727:−;PP][29159:29608:−;PP][29601:29828:−;PP][29954:30379:−;PP][30379:30648:−;PP][30648:30911:−;PP][30911:31144:−;PP][31182:31427:−;PP][31436:31582:−;PP][31569:31724:−;HP][31735:32010:−;PP][32012:32245:−;PP][32377:32523:−;PP][32739:32879:−;PP][33167:33649:−;PP][33653:33958:−;PP][35882:36340:+;PP][36273:36770:+;Phage baseplate hub][36770:38218:+;Phage terminase %2C large subunit][38218:40338:+;Phage portal (connector) protein][40392:40583:+;PP][40583:41575:+;Phage capsid and scaffold][42735:42917:+;PP][42921:43547:+;PP][43558:43749:+;PP][43733:43981:+;PP][43971:44618:+;Phage tail fiber][44627:44725:+;PP].

Essential genes of phage CF1_20Oct199: [2:1267:+;PP][1264:2778:+;PP][2783:5677:+;Phage tail fiber][5679:6107:+;HP][6107:6769:+;Phage endolysin][6794:7045:−;HP][7325:8236:−;DNA ligase %2C phage-associated][8291:8845:−;Phage DNA-binding protein][8842:9447:−;PP][9504:10403:−;PP][10492:11112:−;PP][11207:12766:−;Phage DNA helicase][12763:13173:−,PP][13166:16273:−;DNA polymerase III alpha subunit (EC 2.7.7.7)][18115:19032:−;Thymidylate synthase ThyX (EC 2.1.1.148)][19032:19238:−;HP][19246:19512:−;HP][19512:19730:−;Phage tail assembly protein][19714:19932:−;HP][19932:20162:−;PP][20250:21251:−;HP][21356:22249:−;HP][22410:23597:−;Phage DNA helicase][23584:24006:−;HP][24175:24960:+;HP][25892:26350:+;HP][26347:27423:+;HP][27429:27614:+;HP][27762:29501:+;Phage-associated DNA primase][30469:31038:+;HP][31206:31817:−;HP][32008:32679:−;HP][32931:33242:−;HP][33295:33525:−;HP][33581:33805:−;HP][33870:34196:−;HP][35082:35279:−;HP][35291:35506:−;HP][35503:35703:−;HP][35700:35951:−;HP][36077:36262:−;HP][36347:36535:−;HP][36538:37260:−;Phage capsid and scaffold][37277:37579:−;HP][37934:38098:−;HP][38141:38335:−;HP][38503:39885:+;Phage terminase %2C large subunit][40305:40520:−;HP][40520:40870:−;HP][40914:41297:−;HP][41300:42079:−;HP][43217:43312:−;HP][43309:44241:−;PP][44345:44692:−;HP][45458:45781:−;HP][45813:46214:−;PP][46395:48692:+;PP][48692:49528:+;Phage minor capsid protein][49547:49753:+;PP][49750:49890:+;HP][53735:54172:+;PP][54187:54654:+;PP][54678:55049:+;PP][55057:55608:+;PP][55605:56186:+;PP][56289:57716:+;PP][57775: 58227:+;Phage tail fiber][58227:58550:+;PP][58547:58897:+;PP][58899:59330:+;HP][59340:59843:+;PP][5 9843:60382:+;PP][60391:60984:+;Phage tail fiber][60994:61422:+;HP][61426:64002:+;Phage internal (core) protein][64002:64865:+;PP][64865:65398:+;PP][65454:66119:+;Phage baseplate][66176:66289:+;PP].

Essential genes of phage CF1_20Sep420: [1:855:+;HP][916:1128:+;HP][1128:2102:+;HP][2104:4014:+;Phage DNA helicase (ACLAME 43)][4027:4218:+;HP][4368:4502:+;HP][4512:4802:+;HP][4789:4905:+;HP][4898:5878: +;HP][6080:8137:+;Phage DNA polymerase][8148:8342:+;HP][8437:8841:+;HP][8901:9110:−;HP][9150:9857:−;HP][9858:10154:−;HP][10154:17812:−;HP][17782:18195:−;HP][18195:18707:−;HP][18659:19021:−;HP][19021:21996:−;HP][22059:22496:−;HP][22493:22753:−;HP][22804:23220:−;HP][23237:24913:−;HP][25087:25185:−;HP][25169:25552:−;HP][25549:26058:−;HP][26086:26217:+;HP][26214:26861:+;HP][26866:27330:−;HP][27412:28428:−;Phage major capsid protein of Caudovirales][28406:28504:−;HP][28552:29343:−;HP][29366:30850:−;HP][30854:32467:−;Phage terminase %2C large subunit][32451:32966:−;HP][33021:33563:−;HP][33563:33871:−;HP][33868:34239:−;HP][34236:34703:−;HP][34743:35147:−;HP][35207:35686:−;HP][35683:36249:−;HP][36336:36680:−;HP][36719:36952:−;HP][36954:37244:−;HP][37241:37639:−;HP][37693:37878:−;HP][37875:38216:−;HP][38213:38413:−;HP][38410:38649:−;HP][38649:38921:−;HP][38918:39094:−;HP][39091:39300:−;HP][39303:39536:−;HP][39533:39757:−;HP][39754:40047:−;HP][40058:40231:−;HP][40241:40393:−;HP][40372:40485:−;HP][40574:40783:−;HP][40780:41094:−;HP][41193:41429:−;HP][41426:41716:−;HP][41716:41973:−;HP][41995:42429:−;HP][42520:42927:−;HP][42929:43117:−;HP][43114:43260:−;HP][43276:43494:−;HP][43705:43890:−;HP][43969:44157:−;HP][44356:44448:−;HP][44426:45634:+;HP][45703:45915:+;HP][45924:46673:+;HP][46670:46933:+;HP][46937:47200:+;HP][47202:47462:+;HP][47437:47919:+;HP][47946:48128:+;HP][48200:48493:+;HP][48542:49132:+;HP][49132:49419:+;HP][49424:49615:+;HP][49633:4992 9:+;HP][49935:50798:+;HP][50869:51336:+;HP][51393:51617:+;HP][51628:51972:+;H P][51969:52202:+;HP][52202:52435:+;HP][52512:52934:+;HP][52849:53358:+;HP][53 339:53686:+;HP][53715:53936:+;HP][53981:54241:+;HP][54244:54597:+;HP][54510:5 5046:−;HP][54994:55317:+;HP][55371:55583:+;HP][55580:55744:+;HP][55741:57984:+;HP][57986:58780:+;HP][58855:59265:+;HP][59332:59574:+;HP][5966 9:59899:+;HP][59896:60123:+;HP][60120:60524:+;HP][60527:61474:+;HP][61525:622 47:+;HP][62253:62483:+;HP].

Essential genes of phage CF1_20Sep418: [2:418:−;HP][415:795:−;HP][795:1067:−;HP][1246:1722:−;HP][1773:2807:−;HP][2853:3263:−;HP][3291:4208:−;HP][4205:4675:−;HP][4685:6124:−;HP][6137:7657:−;Phage terminase %2C large subunit][10516:10839:−;HP][11644:12111:+;HP][12108:12464:+;HP][12512:13060:+;HP][13121:13306:+;HP][13303:13596:+;HP][13597:13782:+;HP][1381 7:14095:+;HP][14092:14370:+;HP][14384:14701:+;HP][14710:14979:+;HP][14976:151 88:+;HP][15198:15434:+;HP][15464:15853:+;HP][15850:17058:+;HP][17071:17532:+; HP][17597:18157:+;HP][18159:18719:+;HP][18709:19140:+;HP][19140:19694:+;HP][1 9707:19943:+;HP][19945:20220:+;HP][20217:20624:+;HP][20636:21553:+;HP][21564: 21980:+;HP][21990:22856:+;ribose-phosphate pyrophosphokinase family protein][22853:23071:+;HP][23128:24816:+;Nicotinamide phosphoribosyltransferase (EC 2.4.2.12)][24829:25026:+;HP][25023:25502:+;HP][25514:25663:+;HP][25665:25976:+;HP][25973:26554:+;HP][26938:27441:+;HP][27443:27784:+;HP][2778 1:27996:+;HP][28053:28415:+;HP][28415:28726:+;HP][28714:28992:+;HP][28995:297 02:+;HP][29719:30141:+;HP][30131:30535:+;HP][30535:30801:+;HP][31533:32135:−;HP][32320:32457:+;HP][32949:33107:−;HP][33270:33440:−;HP][33490:33684:−;HP][33784:34050:−;HP][34145:34435:−;HP][34453:34905:−;HP][35440:35991:−;HP][36066:36581:−;HP][36665:37162:−;HP][37199:37513:−;HP][37525:37770:−;HP][37874:38266:−;HP][38263:38529:−;HP][38562:38786:−;HP][38805:38999:−;HP][39263:39616:−;HP][39616:39954:−;HP][39959:40630:−;HP][40706:41089:−;HP][41326:41472:−;HP][41546:41854:−;HP][41854:41982:−;HP][42053:42301:−;HP][42298:42585:−;HP][42588:42728:−;HP][42741:43007:−;HP][43082:43558:−;HP][44187:44360:−;HP][44583:45569:−;HP][46010:46240:−;HP][46242:46436:−;HP][46446:46931:−;HP][46943:47191:−;HP][47232:47417:−;HP][47407:47721:−;HP][47721:47957:−;HP][47957:48190:−;HP][48635:50380:−;Ribonucleotide reductase of class Ia (aerobic) % 2C alpha subunit (EC 1.17.4.1)][50373:51509:−;HP][51436:51780:−;HP][51783:52730:−;putative thymidylate synthase][52785:52877:−;HP][52937:53887:−;HP][53880:54047:−;HP][54096:54431:−;HP][54450:54665:−;HP][54677:54859:−;HP][54856:55635:−;HP][55632:55802:−;HP][55799:56236:−;HP][56233:56463:−;HP][56460:57023:−;HP][57020:58072:−;HP][58114:58335:−;HP][58345:58578:−;HP][58641:59645:−;HP][59747:60463:−;HP][60465:60632:−;HP][60662:61060:−;HP][61150:61839:−;HP][62127:64127:−;HP][64188:66050:−;HP][66104:66289:−;HP][66286:66531:−;HP][66541:66732:−;HP][66719:66847:−;HP][66965:67387:−;HP][67388:67690:−;HP][67693:67857:−;HP][67844:68509:−;HP][68506:68895:−;HP][68897:69151:−;HP][69185:69586:−;HP][69673:69870:−;HP][70580:70813:+;HP][70843:71988:+;PP][72001:72159:+;HP][72152:72961:+;Phage protein (ACLAME 992)][72963:73283:+;HP][73295:73603:+;HP][73651:73965:−;HP][74001:74306:−;HP][74438:74881:−;HP][74868:75107:−;HP][75125:75685:−;Phage endolysin][75702:77201:−;HP][77215:77589:−;HP][77633:79690:−;HP][79701:80432:−;HP][80451:81914:−;HP][81916:82287:−;HP][82298:83038:−;HP][83035:83952:−;HP][83949:84305:−;HP][84311:85072:−;HP][85069:87435:−;tail length tape-measure protein][87432:87584:−;HP][87692:88063:−;HP][88077:88556:−;HP][88556:88927:−;HP][89131:89655:−;HP][89686:90972:−;HP][90985:91164:−;HP].

Essential genes of phage CF1_20Aug400: [1:186:+;Phage structural protein p29][198:1730:+;Phage collar %2C head-to-tail connector protein Gp8][1734:2702:+;Phage capsid assembly scaffolding protein p31][2754:3761:+;Phage major capsid protein Gp10A][3858:4412:+;Phage non-contractile tail tubular protein Gp11][4415:6895:+;Phage non-contractile tail tubular protein Gp12][6895:7440:+;Phage protein p35][7440:10136:+;Phage baseplate hub structural protein/Phage lysozyme R (EC 3.2.1.17)][10140:14153:+;Phage DNA ejectosome component Gpl6%2C peptidoglycan lytic exotransglycosylase (EC 4.2.2.n1)][14155:14910:+;Phage non-contractile tail fiber protein Gp17][14910:15368:+;Phage protein p39][15361:16269:+;Phage protein p40][16273:16878:+;Phage protein p41][16878:17183:+;Phage terminase small subunit Gpl8%2C DNA packaging][17193:18998:+;Phage terminase large subunit Gp19%2C DNA packaging][18998:19195:+;Phage protein p44][19330:19674:+;Phage endolysin][19632:19961:+;Putative phage-encoded lipoprotein p46][20051:20365:+;Phage protein p47][20415:20609:+;Phage protein p48][22644:22928:+;Phage protein p01][22928:23155:+;Phage protein p02][23166:23705:+;Phage protein p03][23768:23872:+;HP][23875:23994:+;HP][24073:24441:+;Phage protein p04][24428:24655:+;Phage protein p05][24834:25007:+;Phage protein p06][25007:25291:+;Phage protein p07][25527:25820:+;Phage protein p07][25899:26312:+;Phage protein p10][26381:26740:+;Phage protein p11][26743:27675:+;Phage DNA-binding protein p12][27937:28479:+;Phage protein p13][28484:28597:+;PP][28656:29489:+;Phage primase/helicase protein Gp4A][29533:30726:+;Phage DNA helicase][30716:31336:+;Phage protein p16][31285:32283:+;Phage-associated ATP-dependent DNA ligase (EC 6.5.1.1)][32280:32600:+;Phage protein p18][32597:35020:+;Phage DNA-directed DNA polymerase (EC 2.7.7.7)][35017:35328:+;Phage protein p20][35383:36432:+;Phage protein p21][36432:37373:+;Phage exonuclease (EC 3.1.11.3)][37363:37803:+;Phage endonuclease][37800:38846:+;Phage exonuclease][38856:39227:+;Phage protein p25][39220:39570:+;PP][39579:42026:+;Phage DNA-directed RNA polymerase (EC 2.7.7.6)][42220:42471:+;Phage protein p27][42471:42944:+;Phage protein p28].

Essential genes of phage CF1_20Dec110: [11:96:−;HP][171:1916:−;Ribonucleotide reductase of class Ia (aerobic) % 2C alpha subunit (EC 1.17.4.1)][1909:3045:−;HP][2972:3316:−;HP][3318:4265:−;putative thymidylate synthase][4320:4412:−;HP][4472:5422:−;HP][5415:5582:−;HP][5631:5966:−;HP][5985:6200:−;HP][6212:6394:−;HP][6391:7170:−;HP][7167:7337:−;HP][7334:7771:−;HP][7768:7974:−;HP][7995:8381:−;HP][8378:8941:−;HP][8938:9990:−;HP][10032:10256:−;HP][10263:10496:−;HP][10559:11563:−;HP][11665:12381:−;HP][12383:12550:−;HP][12580:12978:−;HP][13068:13757:−;HP][14045:16045:−:HP][16106:17968:−;HP][18022:18207:−;HP][18204:18449:−;HP][18459:18650:−;HP][18637:18765:−;HP][18883:19305:−;HP][19306:19608:−;HP][19611:19775:−;HP][19762:20427:−;HP][21103:21504:−;HP][21591:21788:−;HP][22512:22745:+;HP][22775:23920:+;PP][23933:24091:+;HP][24084:24893:+;Phage protein (ACLAME 992)][24895:25215:+;HP][25227:25535:+;HP][25583:25897:−;HP][25933:26238:−;HP][26370:26813:−;HP][26800:27039:−;HP][27057:27617:−;Phage endolysin][27634:29133:−;HP][29147:29521:−;HP][29565:31622:−;HP][31633:32364:−;HP][32383:33846:−;HP][33848:34219:−;HP][34230:34970:−;HP][34967:35884:−;HP][35881:36237:−;HP][36243:37004:−;HP][37001:39367:−;tail length tape-measure protein][39364:39516:−;HP][39624:39995:−;HP][40009:40488:−;HP][40488:40859:−;HP][41063:41587:−;HP][41618:42904:−;HP][42917:43480:−;HP][43477:43857:−;HP][43857:44129:−;HP][44308:44784:−;HP][44835:45869:−;HP][45915:46325:−;HP][46353:47270:−;HP][47267:47737:−;HP][47747:49186:−;HP][49199:50719:−;Phage terminase %2C large subunit][53578:53901:−;HP][54707:55174:+;HP][55171:55527:+;HP][55575:56123:+;HP][56184:56369:+;HP][56366:56659:+;HP][56660:56845:+;HP][56880:57158:+;HP][57155:57433:+;HP][57447:57764:+;HP][57773:58042:+;HP][58039:58251:+;HP][58261:58497:+;HP][58527:5891 6:+;HP][58913:60121:+;HP][60134:60595:+;HP][60660:61220:+;HP][61222:61782:+;H P][61772:62203:+;HP][62203:62757:+;HP][62770:63006:+;HP][63008:63283:+;HP][63 280:63687:+;HP][63699:64616:+;HP][64627:65043:+;HP][65053:65919:+;ribose-phosphate pyrophosphokinase family protein][65916:66134:+;HP][66191:67879:+;Nicotinamide phosphoribosyltransferase (EC 2.4.2.12)][67892:68089:+;HP][68086:68565:+;HP][68577:68726:+;HP][68728:69039:+;HP][69036:69614:+;HP][69998:70501:+;HP][70503:70844:+;HP][7084 1:71056:+;HP][71113:71475:+;HP][71475:71786:+;HP][71774:72052:+;HP][72055:727 62:+;HP][72779:73201:+;HP][73191:73595:+;HP][73595:73840:+;HP][74368:74970:−;HP][76073:76243:−;HP][76571:76861:−;HP][76879:77313:−;HP][77369:77539:−;HP][77564:77701:−;HP][79070:79567:−;HP][79604:79918:−;HP][79930:80175:−;HP][80279:80671:−;HP][80668:80934:−;HP][80967:81191:−;HP][81210:81404:−;HP][81668:82021:−;HP][82021:82359:−;HP][82364:83035:−;HP][83111:83494:−;HP][83731:83877:−;HP][83951:84259:−;HP][84259:84387:−;HP][84458:84706:−;HP][84703:84990:−;HP][84993:85133:−;HP][85146:85412:−;HP][85487:85963:−;HP][86592:86765:−;HP][86988:87974:−;HP][88415:88645:−;HP][88647:88841:−;HP][88851:89336:−;HP][89348:89596:−;HP][89637:89822:−;HP][89812:90126:−:HP][90126:90362:−;HP][90362:90595:−;HP].

Essential genes of phage CF1_21Oct114: [42:258:+;HP][723:945:+;HP][953:1241:+;HP][1243:1591:+;HP][1836:2844:+;HP][3153:3711:+;HP][3842:5699:+;N-acetylneuraminate epimerase][5896:6433:+;HP][7106:7394:+;HP][7393:7609:+;HP][7669:8608:+;N-acetylneuraminate epimerase][8917:10486:+;HP][10678:11242:+;HP][11473:11773:+;HP][11772:12033:+; HP][12040:12217:+;HP][12397:13282:+;HP][13332:13866:−;HP][13878:14007:−;HP][14012:14690:−;HP][15119:15650:−;HP][15636:16104:−;HP][16333:16756:−;HP][16755:17457:−;HP][17535:18225:−;HP][18300:19815:−;ATP-dependent zinc metalloprotease FtsH][19811:20639:−;HP][20718:20922:−;HP][20927:21488:−;HP][21490:21832:−;HP][21828:22038:−;HP][22034:22817:−;HP][23058:23295:−;HP][23336:23852:−;HP][23916:24276:−;HP][24289:24805:−;HP][24818:25187:−;HP][25197:25707:−;HP][25696:26290:−;HP][26299:26761:−;HP][26760:27216:−;HP][27302:27761:−;HP][27753:28197:−;HP][28193:28709:−;HP][28726:29107:−;HP][29163:29643:−;HP][29646:30006:−;HP][30013:30244:−;HP][30251:30896:−;HP][30933:31293:−;HP][31337:31862:−;HP][31876:32074:−;HP][32105:32405:−;HP][32516:32873:−;HP][32966:33407:−;HP][33406:33784:−;HP][33798:34191:−;HP][34199:34460:−;HP][34462:34885:−;HP][34884:35235:−;HP][35289:35763:−;HP][35805:36336:−;HP][36337:36730:−;HP][36726:36963:−;HP][36977:37583:−;HP][37748:38156:−;HP][38207:38630:−;HP][38692:39004:−;HP][39063:39930:−;HP][39926:40277:−;HP][40284:40629:−;HP][40625:40961:−;HP][40942:41158:−;HP][41154:41496:−;HP][41495:41786:−;HP][41785:42286:−;HP][42285:42654:−;HP][42650:43073:−;HP][43069:43414:−;HP][43410:43758:−;HP][43798:44293:−;HP][44285:44480:−;HP][44460:44631:−;HP][44726:45176:−;HP][45190:45640:−;HP][45642:45999:−;HP][46106:46379:−;HP][46378:46984:−;HP][47010:47490:−;HP][47605:47998:−;HP][48008:48347:−;HP][48383:48788:−;HP][48932:49493:−;HP][49482:49962:−;HP][50012:50114:−;HP][50123:50435:−;HP][50687:51257:−;HP][51253:51811:−;HP][51863:52313:−;HP][52315:52597:−;HP][52571:52958:−;HP][53011:54367:−;HP][54368:54575:−;HP][54571:54970:−;HP][55059:56826:−;HP][56842:57730:−;HP][57777:59031:−;RNA-splicing ligase RtcB][59170:59752:−;HP][59748:60576:−;HP][60572:60980:−;HP][60976:61585:−;HP][61632:63063:−;Thymidylate synthase][63062:63401:−;HP][63387:63792:−;HP][63788:64097:−;HP][64093:64360:−;HP][64362:65118:−;HP][65202:65451:−;HP][65450:66248:−;HP][66262:66694:−;HP][66701:67217:−;HP][67339:67705:−;HP][67757:68258:−:HP][68266:68431:−;HP][68430:68856:−;HP][68855:69251:−;HP][69317:69650:−;HP][69627:69978:−;HP][70122:70515:−;HP][70601:71090:−;HP][71134:71344:−;HP][71380:71632:−;HP][71650:73693:−;DNA ligase][73685:74240:−;HP][74337:74805:−;HP][74894:75539:−;dCTP deaminase][75587:76166:−;HP][76152:76461:−;HP][76706:77345:−;HP][77352:77829:−;HP][77830:78568:−;7-cyano-7-deazaguanine synthase][78618:78720:−;HP][78683:78842:−;HP][78851:79274:−;HP][79215:79587:−;HP][79586:80051:−;HP][80060:81161:−;HP][81215:81575:−;HP][81604:82021:−;HP][82017:82800:−;HP][82905:83856:+;HP][83868:84855:+;HP][84857:85808:+;HP][85816:86770:+;HP][87038:90194:+;HP][90206:90593:+;HP][90605:93656:+;HP][9369 3:94365:−;HP][94386:94818:−;HP][94894:95383:−;HP][95488:95851:−;HP][95918:96401:−;HP][96489:97209:−;HP][97268:97499:−;HP][97533:98568:−;Thymidylate kinase][98632:99052:−;HP][99111:99312:−;HP][99321:99816:−;HP][99987:100569:−;HP][100578:101049:−;HP][101065:103042:−;HP][103097:109868:−;HP][109935:111588:+;HP][111590:115952:+;HP][115935:116130:+;HP][116142:11784 9:+;HP][117918:118641:+;HP][118656:119490:+;HP][119539:119851:−;HP][120018:121143:−;HP][121223:121601:−;HP][121605:122361:−;HP][122414:122957:−;HP][123032:123545:+;HP][123588:124224:−;HP][124351:124792:−;HP][124788:124932:−;HP][125056:125449:−;HP][125485:126010:−;HP][126006:126180:−;HP][126172:126529:−;HP][126544:126856:−;HP][126961:127306:−;HP][127336:129829:−;HP][129897:130707:+;HP][130706:130973:+;HP][131013:132198:−;HP][132208:133969:−;HP][134022:134652:−;HP][134728:135184:−;HP][135191:135611:−;HP][135620:137195:−;HP][137242:138550:−;HP][138739:139027:+;HP][139053:139464:+;HP][139506:140952:+;Ribonuclease H][141067:141373:+;HP][141413:142382:−;HP][142480:143920:+;HP][143864:144257:+;HP][144305:144653:+;HP][144675:144996:+;HP][145031:145706:+; HP][145708:146185:+;HP][146181:146943:+;HP][146966:150290:−;HP][150289:152758:−;HP][152925:153945:−;HP][154076:154856:−;HP][154873:155407:−;HP][155497:155902:−;HP][156123:156618:−;HP][156630:157197:−;HP][157207:158104:−;HP][158187:158676:−;HP][158683:159166:−;HP][159110:159707:−;HP][159719:161084:−;HP][161084:162491:−;HP][162501:163869:−;HP][163946:164270:−;HP][164281:166564:−;HP][166658:167939:+;HP][167978:170675:−;HP][170710:172879:+;HP][172881:173760:+;HP][173769:174198:+;HP][174251:17447 9:−;HP][174524:175229:−;HP][175282:175822:−;HP][175871:176282:−;HP][176327:178391:−;HP][178414:180040:−;HP][180048:181086:−;HP][181048:181528:−;HP][181744:183970:−;HP][184041:184578:−;HP][184715:186254:+;HP][186314:186716:+;HP][187131:187398:−;HP][187651:188089:−;HP][188094:188361:−;HP][188360:188603:−;HP][188599:188809:−;HP][188805:189360:−;HP][189520:189805:−;HP][189794:190133:−;HP][190428:191457:−;HP][191547:192180:−;HP][192189:192330:−;HP][192363:193521:−;HP][193577:194180:−;HP][194353:194740:−;HP][194744:195068:−;HP][195106:195259:−;HP][195264:195606:−;HP][195617:195773:−;HP][195888:196170:−;HP][196196:196394:−;HP][196409:196739:−;HP][196735:196831:−;HP][196833:196938:−;HP][197009:197438:−;HP][197512:197986:−;HP][198054:198219:−;HP][198228:198372:−;HP][198379:198811:−;HP][198842:200228:−;HP][200227:200800:−;HP][200806:202213:−;HP][202215:203862:−;HP][203940:206475:−;HP][206569:207691:−;HP][207740:209174:−;HP][209234:210395:−;HP][210469:211732:−;HP][211791:214446:−;HP][214514:215813:−;HP][215823:216357:−;HP][216367:217261:−;HP][217276:218461:−;HP][218498:219542:+;HP][219552:222468:+;HP][222506:223745:−;HP][223737:224040:−;HP][224057:224498:−;HP][224513:225761:−;HP][225869:227813:+;HP][227890:228178:+;HP][228216:228588:−;HP][228574:229486:−;HP][229559:230984:−;HP][231046:232390:−;HP][232376:233228:−;HP][233352:233949:+;HP][233960:235562:+;HP][235627:236023:+;HP][236072:23631 2:−;HP][236325:236586:−;HP][236594:236783:−;HP][236787:236997:−;HP][237044:237347:−;HP][237357:237630:−;HP][237677:238739:−;HP][238774:239212:−;HP][239268:241242:−;HP][241268:242156:−;HP][242440:243394:−;HP][243405:244620:−;HP][244693:245056:+;HP][245099:246419:−;HP][246393:248028:−;HP][248047:249625:−;HP][249747:250494:−;HP][250578:251406:−;HP][251408:252602:−;HP][252543:253155:−;HP][253135:253726:−;HP][253730:254135:−;HP][254203:254626:−;HP][254684:255299:−;HP][255319:256786:−;DNA-directed RNA polymerase subunit beta'][257178:259044:−;HP][259334:260960:−;HP][261041:262127:+;HP][262164:262587:−:HP][262629:263799:−;HP][263853:264018:−;HP][264028:266179:−;HP][266360:266768:−;HP][266791:267217:−;HP][267206:268001:−;HP][268352:268823:−;HP][268764:269109:−;HP][269131:269524:−;HP][269566:270586:−;HP][270602:270995:−;HP][271144:271957:+;HP][271904:272891:+;HP][272938:273169:−;HP][273180:273405:−;HP][273514:273922:−;HP][273928:274357:−;HP][274383:274662:−;HP][274645:275212:−;HP][275257:275752:−;HP][275831:276254:−;HP][276250:276493:−;HP][276539:277487:−;HP][277633:278398:−;HP][278394:278523:−;HP][278533:278755:−;HP][279126:279912:−;HP][280014:280320:−;HP][280329:281091:−;HP][281178:281526:−;HP][281634:282021:−;HP][282024:282651:−;HP][282660:283665:−;HP][283764:285009:+;HP][285059:285275:−;HP][285274:285478:−;HP][285481:285847:−;HP][285858:286218:−;HP][286320:286533:−;HP][286532:287408:−;HP][287440:289534:−;HP][289665:290601:+;HP][290611:293308:+;HP][293309:294974:+;HP][295041:29569 2:+;HP][295778:297965:+;HP][298008:298503:−;HP][298534:299056:−;Dihydrofolate reductase][299067:299649:−;HP][299645:299999:−;HP][300195:302481:+;Ribonucleoside-diphosphate reductase 1 subunit alpha][302610:303768:+;Ribonucleoside-diphosphate reductase 1 subunit beta][303767:304181:+;HP].

### Example 8 Synergic Increased TTM Achieved by a Phage Cocktail

To test the effect on the time till growth of resistant mutant bacteria (TTM) is detected, cocktails CFX1 and CFX7 as well as individual member phages of those cocktails were tested against different bacterial strains. 10 bacterial colonies of each bacterial strain tested were picked (˜full 1 uL loop) and transferred into a culture tube prefilled with 4 mL of liquid BHIS and cultured to OD600≥1.5 by shaking, 180 rpm, at 37° C. for ˜16 h. The bacterial culture was diluted using BHIS supplemented with 1 mM MMC ions to reach a final OD of 0.05 and dispensed into a 96-well plate. Each phage was diluted to a concentration of 10\hat{ }8 PFU/ml, and to create the cocktail, equal ratios were mixed to get the same total concentration as the individuals. Then, 10 μL of the sample of single or cocktail phages were added to the wells to a final concentration of 10\hat{ }6 PFU/well. For NPC, BHIS was added to the appropriate wells. Mineral oil was added to each well to reduce evaporation of the samples, and the plate was covered with sterile film to allow bacteria growth and keep the culture sterile. Plates were incubated for 30-45 hours in a plate reader at 37° C. with shaking, and OD600 was measured every 20 minutes. Two biological repeats were performed for the assay, and BHIS media supplemented with 1 mM MMC ions served as a blank. FIGS. 5A to 5D present the effect of CFX7 cocktail compared to each member phage with respect to four bacterial strains, 788, 908, 560 and 667, respectively. For example, with respect to bacterial strain 788, elimination of mutant growth is achieved up till 25 hours from experiment start, and in case of bacterial strain 667 growth of mutant bacteria was eliminated up till end of the experiment. FIGS. 5E to 5F present the effect of CFX1 cocktail compared to each member phage with respect to two bacterial strains, 830 and 907, respectively. FIG. 5G present the effect of CFX1 cocktail compared to each member phage with respect to a mixture of equal concentration of three bacterial strains, prepared as described above. The ability of CFX1 to eliminate any bacterial resistant mutants' growth for prolong time is achieved.

Altogether, FIGS. 5A to 5G demonstrates the ability of a synergistic performing phage mixture to eliminate resistant mutant growth in comparison each phage member separately.

Table 5 presents, for each graph in FIGS. 5A to 5G, the approximate time (in hours) when the corresponding OD600 reading reaches the value 0.1, indicative of mutant bacterial growth. The table also presents the OD600 normalized area under the curve (AUC), i.e., the ratio between the AUC600 of the line representing treatment with the phage and the AUC600 of the line representing OD600 readings of the no phage control (NPC):

$$normalized area under the curve = \frac{Phage treatment area under the curve}{NPC area under the curve}$$

TABLE 5

| | Approx. Time (hours) | Normalized area under |
|---|---|---|
| | when OD600 reaches 0.1 | the curve |
| 788 + CF1_20Nov10 | 18.9 | 0.39 |
| 788 + CF1_20Dec107 | 16.7 | 0.42 |
| 788 + CF1_20Dec110 | 17.4 | 0.30 |
| 788 + CF1_21Oct114 | 34.1 | 0.16 |
| 788 + CFX7 | No mutant's growth | 0.04 |
| 788 | 3.0 | 1.00 |
| 908 + CF1_20Nov10 | 9.8 | 0.81 |
| 908 + CF1_20Dec107 | 12.1 | 0.73 |
| 908 + CF1_20Dec110 | 13.6 | 0.59 |
| 908 + CF1_21Oct114 | 28.0 | 0.24 |
| 908 + CFX7 | No mutant's growth | 0.08 |
| 908 | 2.3 | 1.00 |
| 560 + CF1_20Nov10 | 10.6 | 0.57 |
| 560 + CF1_20Dec107 | 13.6 | 0.52 |
| 560 + CF1_20Dec110 | 12.1 | 0.45 |
| 560 + CF1_21Oct114 | 25.0 | 0.27 |
| 560 + CFX7 | No mutant's growth | 0.11 |
| 560 | 3.0 | 1.00 |
| 667 + CF1_20Nov10 | 12.1 | 0.71 |
| 667 + CF1_20Dec107 | 12.9 | 0.51 |
| 667 + CF1_20Dec110 | 13.6 | 0.54 |
| 667 + CF1_21Oct114 | 34.8 | 0.17 |
| 667 + CFX7 | No mutant's growth | 0.03 |
| 667 | 3.0 | 1.00 |
| 830 + CF1_20Nov10 | 12.9 | 0.68 |
| 830 + CF1_20Dec107 | 0.8 | 0.57 |
| 830 + CF1_20Dec110 | 18.2 | 0.42 |
| 830 + CF1_21Oct114 | 35.6 | 0.25 |
| 830 + CFX1 | 22.7 | 0.30 |
| 830 + CFX7 | 39.4 | 0.23 |
| 830 | 0.1 | 1.00 |
| 907 + CF1_20Nov10 | 16.7 | 0.48 |
| 907 + CF1_20Dec107 | 3.0 | 0.90 |
| 907 + CF1_20Dec110 | 15.9 | 0.42 |
| 907 + CF1_21Oct114 | No mutant's growth | 0.21 |
| 907 + CFX1 | 25.8 | 0.28 |
| 907 + CFX7 | No mutant's growth | 0.13 |
| 907 | 3.0 | 1.00 |
| Bacteria + CFX1 | 25.8 | 0.31 |
| Bacteria + CF1_20Dec110 | 8.3 | 1.02 |
| Bacteria + CF1_20Dec107 | 10.6 | 0.67 |
| Bacteria + CF1_20Nov10 | 9.8 | 1.15 |
| Bacteria only | 8.3 | 1.00 |

### Example 9 Testing the Phage Efficacy Using In Vivo and Ex Vivo Models of Chronic Lung Infection with Pseudomonas aeruginosa

Animal models of cystic fibrosis (CF) are used to assess phage efficacy in relevant niche that mimics human CF lungs. For example, Kent and colleagues reported the development of a congenic strain designated B6-CFTRtm1UNC/CFTRtm1UNC (Kent G et al., 1997, Guilbault et al., 2006, Zhou et al., 2011), which enable spontaneous and progressive lung disease. In addition, Transgenic mice overexpressing airway-specific ENaC to increase Na ions absorption, enable spontaneous and progressive lung disease (Kukavica-Ibrulj et al., 2008). Ex vivo systems include the CF bronchial epithelial co-culture model for P. aeruginosa biofilms (Moreau-Marquis, Bomberger, et al. 2008, AJP Lung) where the bronchial epithelial cells of a CF patient are co-cultured with P. aeruginosa biofilms to mimic CF lung niche and to enable testing of various anti P. aeruginosa treatments.

Such models are used to test and confirm the phages' efficacy e.g., by measuring if the bacterial burden & biofilm mass are reduced upon phage treatment.

### Example 10 Phage Specificity

To verify the specificity of the phages, three phages, CF1_20Nov10, CF1_20Dec107 and CF1_20Dec110 were tested against other species of bacteria as detailed in the table below. A solid assay was used as detailed above. Bacterial strains on which the EOP was ≥0.1 for the tested phage (i.e., sensitivity to the tested phage) were designated as “S” within green cells in all results tables. Bacteria on which the EOP was <0.1 for the tested phage were designated as resistant to the tested phage (“R” within red cells in all tables). No cross-species infectivity was observed.

| Bacteria | CF1_20Nov10 | CF1_20Dec107 | CF1_20Dec110 |
|---|---|---|---|
| Veillonella dispar | R | R | R |
| Staphylococcus aureus | R | R | R |
| Fusobacterium nucleatum | R | R | R |
| Klebsiella aeruginosae | R | R | R |
| Escherichia coli | R | R | R |
| Pseudomonas aeruginosa | S | S | S |
| (positive control) | | | |

### Example 11 Phage Efficacy in CF Patients' Sputum

To evaluate the ability of the phage to infect bacteria in a clinically relevant sample matrix: sputum samples from CF patients, a cocktail of CF1_20Nov10, CF1_20Dec107 and CF1_20Dec110 was added to two sputum samples derived from CF patients and spiked with known amounts of a bacterial strain which is sensitive to this cocktail. The titer of the phages in the different sputum samples was measured following overnight (O/N) incubation. An increase in phage titer represents successful infection and amplification of the phage within the bacteria. An increase in phage titer, above 1 log was detected when the sputum samples were spiked with the known sensitive bacteria and the cocktail of phages, at an MOI of 1. When the phage cocktail was added to the sputum sample without bacterial spiking, no increase in PFU was observed when compared to the initial PFU levels. No PFUs were detected in sputum samples spiked with bacteria but without addition of phages.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

It is the intent of the applicant(s) that all publications, patents, and patent applications referred to in this specification are to be incorporated in their entirety by reference into the specification, as if each individual publication, patent or patent application was specifically and individually noted when referenced that it is to be incorporated herein by reference. In addition, citation, or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A composition comprising at least two different strains of isolated bacteriophages, each capable of (lytically) infecting a bacteria of the species Pseudomonas aeruginosa (e.g., Pseudomonas aeruginosa present in a Cystic Fibrosis patient), wherein at least one of said at least two different strains of isolated bacteriophages has (i) a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to one of the nucleic acid sequence as set forth in SEQ ID NOs: 1-10, and/or (ii) at least 90% (e.g., at least 91%, 92%, 93%, 94%, 95%, 96%, 97%, 98%, 99%, 99.2%, 99.4%, 99.6%, 99.8%, 99.9% or 100%) identical genes (e.g., in the combined region) to the essential genes of a bacteriophage selected from the bacteriophages listed in Table 2, as set forth in Example 7; and
wherein optionally, said at least two different strains of isolated bacteriophages have synergistic redundancy effect, based on either (i) time-to-mutant (TTM) that is at least 10%, 20%, 30%, 40%, 50%, 60%, 70%, or 80% above the longest individual phage TTM with respect to said bacteria, or (ii) normalized area under the curve for OD600-time plot (AUC) that is at least 10%, 20%, 30%, 40%, 50%, 60%, 70%, 80% or 90% smaller than the smallest individual phage normalized area under the curve with respect to said bacteria (or a mixture of more than one of said bacteria).

2. The composition of claim 1, wherein a first of said at least two different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to the nucleic acid sequence as set forth in SEQ ID NO: 1 and a second of said at least two different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to the nucleic acid sequence as set forth in SEQ ID NO: 2.

3. The composition of claim 2, comprising at least three different strains of isolated bacteriophages, wherein a third of said at least three different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to the nucleic acid sequence as set forth in SEQ ID NO: 3.

4. The composition of claims 1 or 2, comprising:
(i) a bacteriophage having a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to SEQ ID NO: 1;
(ii) a bacteriophage having a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to SEQ ID NO: 2;
(iii) a bacteriophage having a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to SEQ ID NO: 3;
(iv) a bacteriophage having a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to SEQ ID NO: 4.

5. The composition of claim 1, wherein said at least two different strains of isolated bacteriophages in combination target at least 40, 45, 50, 55, 60 or 65 different strains of Pseudomonas aeruginosa from the list in Example 1 of Pseudomonas aeruginosa.

6. The composition of claim 1 or 5, wherein at least 25 different strains of Pseudomonas aeruginosa from the list in Example 1 and/or at least 36 different MLSTs of Pseudomonas aeruginosa from the list in FIG. 2 are targeted by each of said at least two different strains.

7. The composition of claim 1, 5, or 6, comprising at least three different strains of isolated bacteriophages, each capable of infecting a bacteria of the species Pseudomonas aeruginosa, wherein each of said at least three different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to one of the nucleic acid sequence as set forth in SEQ ID NOs: 1-10, wherein said at least three different strains of isolated bacteriophages in combination target (i) at least 70 different strains of Pseudomonas aeruginosa from the list in Example 1; and/or (ii) at least 52 different MLSTs of Pseudomonas aeruginosa from the list in FIG. 2.

8. The composition of claim 1, 5, 6, or 7, comprising at least three different strains of isolated bacteriophages, each capable of infecting a bacteria of the species Pseudomonas aeruginosa, wherein each of said at least three different strains of isolated bacteriophages has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to one of the nucleic acid sequence as set forth in SEQ ID NOs: 1-10, wherein (i) at least 40 different strains of Pseudomonas aeruginosa from the list in Example 1, and/or (ii) at least 52 different MLSTs of Pseudomonas aeruginosa from the list in FIG. 2 are targeted by at least two of said at least three different strains.

9. The composition of any one of claims 1-8, wherein said at least one bacteriophage is genetically modified such that the genome thereof comprises a heterologous sequence.

10. The composition of claim 9, wherein said heterologous sequence encodes a therapeutic agent or a diagnostic agent.

11. The composition of any one of claims 1-10, comprising no more than 10 different bacteriophage strains.

12. The composition of any one of claims 1-11, being formulated for oral delivery, rectal delivery or delivery by inhalation.

13. A recombinant bacteriophage capable of infecting bacteria of the species Pseudomonas aeruginosa, wherein said bacteriophage has a genomic nucleic acid sequence at least 90% (e.g., at least 91%, 92%, 93%, 94%, 95%, 96%, 97%, 98%, 99%, 99.2%, 99.4%, 99.6%, 99.8%, 99.9% or 100%) identical (e.g., in the combined coding region) to one of the nucleic acid sequences as set forth in SEQ ID NOs: 1-10, and wherein said bacteriophage is genetically modified such that the genome thereof comprises a heterologous sequence.

14. The recombinant bacteriophage of claim 13, wherein said heterologous sequence encodes a therapeutic agent or a diagnostic agent.

15. The recombinant bacteriophage of claim 14, or the composition of claim 10, wherein said therapeutic agent comprises an immune modulating agent.

16. A pharmaceutical composition comprising the recombinant bacteriophage of claims 13 or 14 as the active agent, and a pharmaceutical carrier.

17. The pharmaceutical composition of claim 16, being formulated for oral delivery, rectal delivery or delivery by inhalation.

18. An isolated bacteriophage capable of (lytically) infecting bacteria of the species Pseudomonas aeruginosa (e.g., Pseudomonas aeruginosa present in a Cystic Fibrosis patient), wherein said bacteriophage has a genomic nucleic acid sequence at least 95% (e.g., at least 95%, 96%, 97%, 98%, 99%, 99.2%, 99.4%, 99.6%, 99.8%, 99.9% or 100%) identical (e.g., in the combined coding region) to one of the nucleic acid sequences as set forth in SEQ ID NOs: 1-10.

19. A method of treating a disease associated with a Pseudomonas aeruginosa infection in a subject in need thereof (e.g., a subject having Cystic Fibrosis), comprising administering to the subject a therapeutically effective amount of a composition comprising at least one isolated bacteriophage strain capable of infecting bacteria of the species Pseudomonas aeruginosa causing the infection, wherein said at least one bacteriophage strain has a genomic nucleic acid sequence at least 95% (e.g., at least 95%, 96%, 97%, 98%, 99%, 99.2%, 99.4%, 99.6%, 99.8%, 99.9% or 100%) identical (e.g., in the combined coding region) to one of the nucleic acid sequences set forth in SEQ ID NOs: 1-10, thereby treating the disease associated with a Pseudomonas aeruginosa infection.

20. A method of treating a disease (e.g., Cystic Fibrosis) associated with a Pseudomonas aeruginosa infection in a subject in need thereof, comprising administering to the subject a therapeutically effective amount of the composition of any one of claims 1-12, thereby treating the disease associated with a Pseudomonas aeruginosa infection.

21. The method of claim 19 or 20, wherein the disease is Cystic Fibrosis (CF).

22. The method of any one of claims 19-21, wherein said administering comprises orally administering or rectally administering.

23. The method of claim 19, wherein said composition comprises no more than 10 different bacteriophage strains.

24. The method of any one of claims 19-23, further comprising identifying the strain of Pseudomonas aeruginosa colonizing the subject prior to the administering.

25. The method of any one of claims 19-24, wherein said at least one bacteriophage strain is genetically modified such that the genome thereof comprises a heterologous sequence.

26. The method of claim 25, wherein said heterologous sequence encodes a therapeutic agent or a diagnostic agent.

27. The method of claim 26, wherein said therapeutic agent comprises an immune modulating agent.

28. The method of any one of claims 19-27, wherein the subject has been treated with, or is to be further treated with an antibiotic effective against Pseudomonas aeruginosa (e.g., Pseudomonas aeruginosa present in a Cystic Fibrosis patient).

29. The method of any one of claims 19-27, further comprising treating the subject with an antibiotic effective against Pseudomonas aeruginosa (e.g., Pseudomonas aeruginosa present in a Cystic Fibrosis patient).

30. The method of claim 28 or 29, wherein the antibiotic comprises aztreonam, colistin, and/or tobramycin.
